(19) United States
(12) Patent Application Publication
(10) Pub. No.: US 2026/0098014 A1
(43) Pub. Date: Apr. 9, 2026

(54) IONIZABLE LIPIDOID COMPOSITIONS AND THERAPEUTIC USES THEREOF

(71) Applicant: Flagship Pioneering Innovations VII, LLC, Cambridge, MA (US)

(72) Inventors: Srinivas Abbina, Melrose, MA (US); Chelsea RamEl Martinez, Cambridge, MA (US); Vandan Paresh Jakharia, Allston, MA (US); Timothy Ray Blake, New York, NY (US); Ulugbek Barotov, Everett, MA (US)

(21) Appl. No.: 19/352,724

(22) Filed: Oct. 8, 2025

Related U.S. Application Data
(60) Provisional application No. 63/704,786, filed on Oct. 8, 2024.

(51) Int. Cl. C07D 233/36; A61K 9/51; A61K 48/00; C07C 229/24; C07D 205/04; C07D 207/09; C07D 211/26; C07D 233/61; C07D 239/10; C07D 295/13; C07D 317/38; C07F 9/6574

(52) U.S. Cl. CPC C07D 233/36; A61K 9/5123; A61K 48/0041; C07C 229/24; C07D 205/04; C07D 207/09; C07D 211/26; C07D 233/61; C07D 239/10; C07D 295/13; C07D 317/38; C07F 9/65742; C07C 2601/08

(57) ABSTRACT

Disclosed are lipidoid compounds having the structure of formula (I):

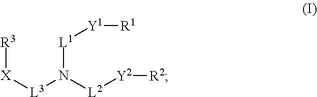

or a salt thereof, wherein the groups are as defined in the application. Also disclosed are nanoparticle compositions comprising a lipidoid of the invention that are capable of delivering a therapeutic agent. The application also discloses pharmaceutical compositions comprising a lipidoid composition of the invention.

### RELATED APPLICATIONS

This application claims the benefit of priority to U.S. Provisional Patent Application No. 63/704,786, filed Oct. 8, 2024, the contents of which are incorporated by reference herein.

### BACKGROUND

The effective targeted delivery of biologically active substances such as small molecule drugs, proteins, and nucleic acids represents a continuing medical challenge. In particular, the delivery of nucleic acids to cells is made difficult by their relative instability and low cell permeability. Thus, there exists a need to develop methods and compositions to facilitate the delivery of therapeutic and/or prophylactics, such as nucleic acids, to cells.

Lipid-containing nanoparticle compositions, liposomes, and lipoplexes can be effective transport vehicles into cells and/or intracellular compartments for biologically active substances such as small molecule drugs, proteins, and nucleic acids. Such compositions generally include one or more cationic and/or ionizable lipids, phospholipids including polyunsaturated lipids, structural lipids (e.g., sterols), and/or lipids containing polyethylene glycol (PEG lipids). There is a need to develop lipidoids useful in lipid-nanoparticle compositions that can deliver therapeutic agents, such as nucleic acid molecules, proteins, and small molecule drugs with safety, efficacy, and specificity.

### SUMMARY

In certain aspects, provided herein is a lipidoid having the structure of formula (I):

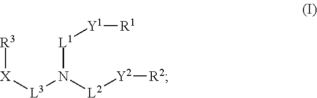

or a salt thereof;
wherein:
each of L¹, L², and L³ represents —(CH₂)ₘ—;
m is independently for each occurrence an integer selected from 1-20;
Y¹ is

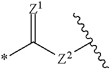

and * represents the connection to L¹;
Y² is

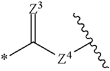

and * represents the connection to L²;
Z¹, Z², Z³, and Z⁴ are each independently O, S, or NH;
X is optionally substituted saturated or unsaturated cycloalkylene, heterocyclylene, heteroarylene, or arylene;
R¹ and R² are each independently optionally substituted linear (C₄-C₂₀)alkyl, branched (C₄-C₂₀)alkyl, linear (C₄-C₂₀)alkenyl, branched (C₄-C₂₀)alkenyl, linear (C₄-C₂₀)alkynyl, or branched (C₄-C₂₀)alkynyl, optionally wherein two substituents, together with the intervening atoms, form an aryl or 5-7 membered cycloalkyl ring; and
R³ is hydrogen, hydroxyl, hydroxyalkyl, amido, or an optionally substituted linear C₂-C₁₀ alkyl or branched C₃-C₁₀ alkyl.

In certain aspects, at least one of Y¹ or Y² is *—(C═O)O—. In certain aspects, Y¹ is *—(C═O)O—. In certain aspects, Y² is *—(C═O)O—.

In certain aspects, Y¹ and Y² are different. In certain other aspects, Y¹ and Y² are the same.

In certain aspects, the lipidoid has the structure of formula (II):

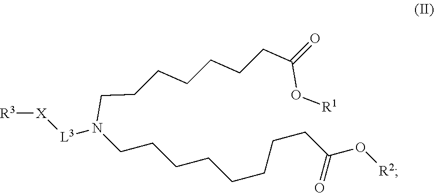

or a salt thereof;
wherein:
L³ is —(CH₂)ₘ—;
m is an integer selected from 1-10;
X is an optionally substituted saturated or unsaturated cycloalkylene, heterocyclylene, heteroarylene, or arylene;
R¹ and R² are each independently optionally substituted linear (C₄-C₂₀)alkyl, branched (C₄-C₂₀)alkyl, linear (C₄-C₂₀)alkenyl, branched (C₄-C₂₀)alkenyl, linear (C₄-C₂₀)alkynyl or branched (C₄-C₂₀)alkynyl, optionally wherein two substituents, together with the intervening atoms, form an aryl or 5-7 membered cycloalkyl ring; and
R³ is hydrogen, hydroxyl, hydroxyalkyl, amido, or an optionally substituted linear C₂-C₁₀ alkyl or branched C₃-C₁₀ alkyl.

In certain aspects, the lipidoid has the structure of formula (III):

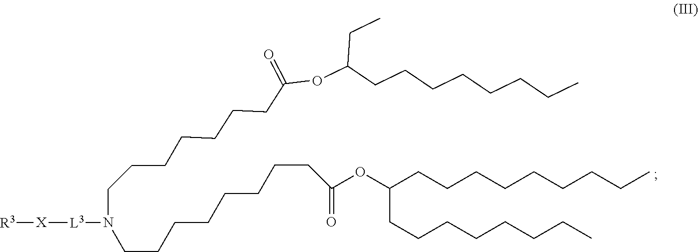

or a salt thereof;
wherein:
L³ is —(CH₂)ₘ—;
m is an integer selected from 1-10;
X is an optionally substituted saturated or unsaturated cycloalkylene, heterocyclylene, heteroarylene, or arylene; and
R³ is hydrogen, hydroxyl, hydroxyalkyl, amido, or an optionally substituted linear C₂-C₁₀ alkyl or branched C₃-C₁₀ alkyl.

In certain aspects, the lipidoid has the structure of formula (IV):

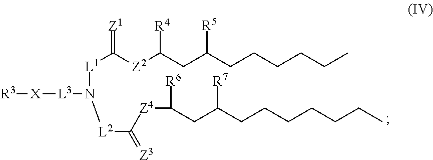

or a salt thereof;
wherein:
each of L¹, L², and L³ represent —(CH₂)ₘ—;
m is independently for each occurrence an integer selected from 1-20;
Z¹, Z², Z³, and Z⁴ are each independently O, S, or NH;
X is an optionally substituted saturated or unsaturated cycloalkylene, heterocyclylene, heteroarylene, or arylene;
R³ is hydrogen, hydroxyl, hydroxyalkyl, amido, or an optionally substituted linear C₂-C₁₀ alkyl or branched C₃-C₁₀ alkyl.

R⁴ and R⁶ are each independently H, linear C₂-C₁₀ alkyl or branched C₃-C₁₀ alkyl;
R⁵ is H, linear C₂-C₁₀ alkyl, or branched C₃-C₁₀ alkyl,
or R⁵ and R⁴, taken together with the intervening atoms, form an aryl or a 5- to 7-membered cycloalkyl; and
R⁷ is H, linear C₂-C₁₀ alkyl, or branched C₃-C₁₀ alkyl,
or R⁷ and R⁶, taken together with the intervening atoms, form an aryl or a 5- to 7-membered cycloalkyl.

In certain aspects, the lipidoid has the structure of formula (V):

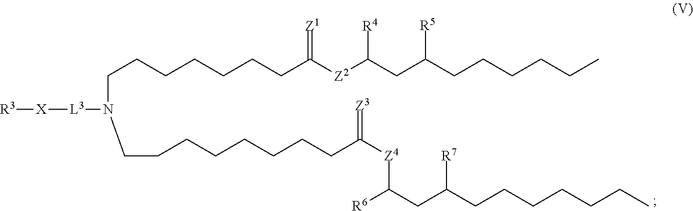

or a salt thereof;
wherein:
L³ represents —(CH₂)ₘ—;
m is an integer selected from 1-10;
each of Z¹, Z², Z³, and Z⁴ are independently O, S, or NH;
X is an optionally substituted saturated or unsaturated cycloalkylene, heterocyclylene, heteroarylene, or arylene;
R³ is hydrogen, hydroxyl, hydroxyalkyl, amido, or an optionally substituted C₂-C₁₀ alkyl linear or branched C₃-C₁₀ alkyl.

R⁴ and R⁶ are each independently H, linear C₂-C₁₀ alkyl or branched C₃-C₁₀ alkyl;
R⁵ is H, linear C₂-C₁₀ alkyl, or branched C₃-C₁₀ alkyl,
or R⁵ and R⁴, taken together with the intervening atoms, form an aryl or a 5- to 7-membered cycloalkyl; and
R⁷ is H, linear C₂-C₁₀ alkyl, or branched C₃-C₁₀ alkyl,
or R⁷ and R⁶, taken together with the intervening atoms, form an aryl or a 5- to 7-membered cycloalkyl.

In certain aspects, X is a five-membered saturated or unsaturated heterocyclylene.

In further aspects, X represents a saturated or partially unsaturated heterocyclylene comprising two heteroatoms.

In certain aspects, X represents a saturated or partially unsaturated heterocyclylene substituted with oxo (═O).

In further aspects, X is

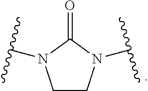 [SMILES: *N1CCN(*)C1=O]

In certain aspects, X comprises a five-membered heteroarylene.

In certain aspects, X comprises a six-membered arylene.

In certain aspects, X comprises a four to seven-membered heterocyclylene.

In certain aspects, X comprises a four to seven-membered cycloalkylene.

In certain aspects, —X—R³ is selected from:

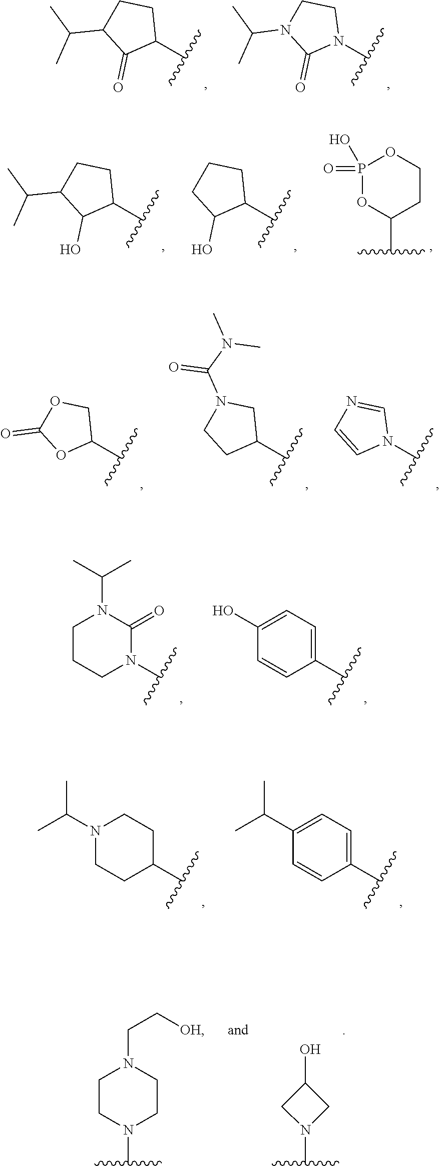

In certain aspects, L³ is —(CH₂)₃—.

In certain aspects, the lipidoid has the structure of formula (VI);

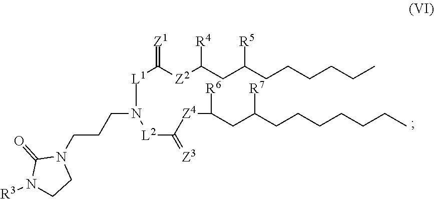

or a salt thereof;
wherein:
each of L¹ and L² represent —(CH₂)ₘ—;
m is independently for each occurrence an integer selected from 1-20;
each of Z¹, Z², Z³, and Z⁴ are independently O, S, or NH;
R³ is hydrogen, hydroxyl, hydroxyalkyl, amido, or an optionally substituted linear C₂-C₁₀ alkyl or branched C₃-C₁₀ alkyl.
R⁴ and R⁶ are each independently H, linear C₂-C₁₀ alkyl or branched C₃-C₁₀ alkyl;
R⁵ is H, linear C₂-C₁₀ alkyl, or branched C₃-C₁₀ alkyl,
or R⁵ and R⁴, taken together with the intervening atoms, form an aryl or a 5- to 7-membered cycloalkyl; and
R⁷ is H, linear C₂-C₁₀ alkyl, or branched C₃-C₁₀ alkyl,
or R⁷ and R⁶, taken together with the intervening atoms, form an aryl or a 5- to 7-membered cycloalkyl.

In certain aspects, m is independently for each occurrence an integer selected from 2-10.

In certain aspects, at least one of L¹ and L² is —(CH₂)₇—.

In certain aspects, at least one of L¹ and L² is —(CH₂)₈—.

In certain aspects, L¹ and L² are the same.

In certain aspects, L¹ and L² are different.

In certain aspects, the lipidoid has the structure of formula (VII);

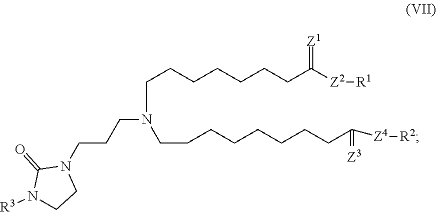

or a salt thereof;
wherein:
R³ is hydrogen, hydroxyalkyl, amido, or an optionally substituted linear C₂-C₁₀ alkyl or branched C₃-C₁₀ alkyl;
each of Z¹, Z², Z³, and Z⁴ are independently O, S, or NH;
R¹ and R² are each independently optionally substituted linear (C₄-C₂₀)alkyl, branched (C₄-C₂₀)alkyl, linear (C₄-C₂₀)alkenyl, branched (C₄-C₂₀)alkenyl, linear (C₄-C₂₀)alkynyl, or branched (C₄-C₂₀)alkynyl, optionally wherein two substituents, together with the intervening atoms, form an aryl or 5-7 membered cycloalkyl ring.

In certain aspects, R³ is isopropyl.

In certain aspects, R¹ and R² are the same.

In certain aspects, R¹ and R² are different.

In certain aspects, R¹ and R² are each independently branched (C₁₁-C₂₀)alkyl.

In certain aspects, R¹ is

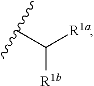

and R^{1a} and R^{1b} are each independently (C₂-C₁₈)alkyl, wherein R¹ contains from 8 to 20 total carbon atoms.

In certain aspects, R^{1a} and R^{1b} are the same.

In certain aspects, R^{1a} and R^{1b} are different.

In certain aspects, R² is

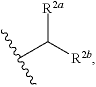

and R^{2a} and R^{2b} are each independently (C₂-C₁₈)alkyl, wherein R² contains from 8 to 20 total carbon atoms.

In certain aspects, R^{2a} and R^{2b} are the same.

In certain aspects, R^{2a} and R^{2b} are different.

In certain aspects, R¹ is:

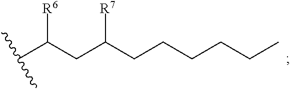

and
R⁴ and R⁵ are each independently H, C₂-C₁₀ alkyl, or R⁴ and R⁵, taken together with the intervening atoms, form an aryl or a 5- to 7-membered cycloalkyl.

In certain aspects, R² is:

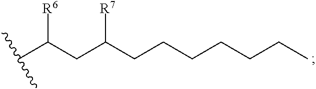

and
R⁶ and R⁷ are each independently H, C₂-C₁₀ alkyl, or R⁶ and R⁷, taken together with the intervening atoms, form an aryl or a 5- to 7-membered cycloalkyl.

In certain aspects, the lipidoid has the structure of formula (VIII):

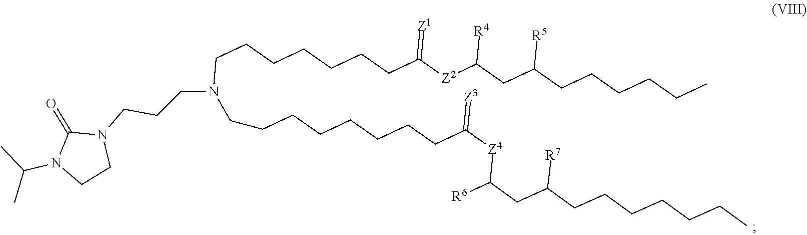

or a salt thereof;
wherein:
each of Z¹, Z², Z³, and Z⁴ are independently O, S, or NH;
R⁴ and R⁶ are each independently H, linear C₂-C₁₀ alkyl or branched C₃-C₁₀ alkyl;
R⁵ is H, linear C₂-C₁₀ alkyl, or branched C₃-C₁₀ alkyl,
   or R⁵ and R⁴, taken together with the intervening atoms, form an aryl or a 5- to 7-membered cycloalkyl; and
R⁷ is H, linear C₂-C₁₀ alkyl, or branched C₃-C₁₀ alkyl,
   or R⁷ and R⁶, taken together with the intervening atoms, form an aryl or a 5- to 7-membered cycloalkyl.

In certain aspects, Z¹, Z², Z³, and Z⁴ are each O.

In certain aspects, the lipidoid is:

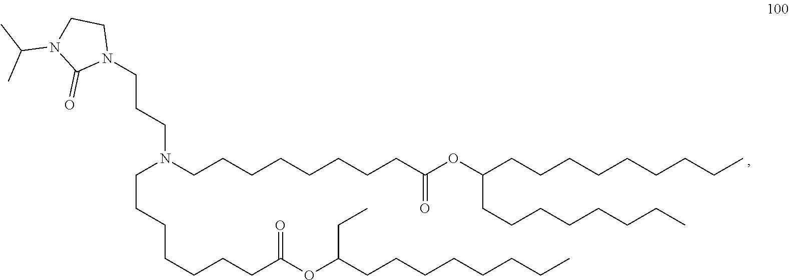 [SMILES: CCCCCCCCCC(CCCCCCCC)OC(=O)CCCCCCCCN(CCCCCCCC(=O)OC(CC)CCCCCCCC)CCCN1CCN(C(C)C)C1=O]

or a salt thereof.

In further aspects, provided herein is a nanoparticle composition, comprising a plurality of a lipidoid of the invention, or a pharmaceutically acceptable salt thereof.

In certain aspects, the nanoparticle composition further comprises a lipid.

In certain aspects, the lipid is a cationic, anionic, ionizable, or zwitterionic lipid.

In certain aspects, the nanoparticle composition further comprises a PEGylated lipid, a sterol, a phospholipid, and/or a neutral lipid.

In certain aspects, the nanoparticle composition is substantially free of a phospholipid. In certain aspects, the phospholipid is distearolyphosphatidycholine (DSPC).

In certain aspects, the nanoparticle composition further comprises a PEGylated lipid, a sterol, and a phospholipid.

In certain aspects, the nanoparticle composition further comprises a therapeutic agent.

In certain aspects, the therapeutic agent is a nucleic acid molecule.

In certain aspects, the nucleic acid molecule is a plasmid, an immunostimulatory oligonucleotide, an antisense oligonucleotide, an antagomir, an aptamer, a deoxyribozyme (DNAzyme), or a ribozyme.

In other aspects, the nucleic acid molecule is DNA or RNA. In some aspects, the nucleic acid molecule is DNA; and the DNA is a linear DNA, circular DNA, single stranded DNA, or double stranded DNA.

In certain aspects, the nucleic acid molecule is RNA; and the RNA is messenger RNA (mRNA), miRNA, siRNA or siRNA precursor, RNA aptamer, linear RNA, circular RNA, single stranded RNA, double stranded RNA, tRNA, microRNA (miRNA) or miRNA precursor, Dicer substrate small interfering RNA (dsiRNA), Dicer substrate RNA (dsRNA), short hairpin RNA (shRNA), asymmetric interfering RNA (aiRNA), guide RNA (gRNA), lncRNA, ncRNA, sncRNA, rRNA, snRNA, piRNA, snoRNA, snRNA, scaRNA, exRNA, scaRNA, Y RNA, or hnRNA.

In certain aspects, the nucleic acid molecule comprises one or more nucleic acid analogs, and each of the one or more nucleic acid analogs is independently a phosphoramide, a phosphorothioate, a phosphorodithioate, an O-methylphosphoroamidate, a morpholino, a locked nucleic acid (LNA), a glycerol nucleic acid (GNA), a threose nucleic acid (TNA), or a peptide nucleic acid (PNA).

In certain aspects, the therapeutic agent is a protein or small molecule drug.

In certain aspects, the nanoparticle composition further comprises an antigen; wherein the antigen is a protein or a nucleic acid; the antigen is a protein; or the antigen is a nucleic acid.

In certain aspects, the nanoparticle composition further comprises an mRNA molecule that encodes an antigen.

In certain aspects, the lipid nanoparticle has a N:P ratio of about 5 to about 15.

In certain aspects, the lipid nanoparticle has a N:P ratio of about 7 to about 12.

In further aspects, the lipid nanoparticle has a N:P ratio of about 10.

In certain aspects, the lipid nanoparticle comprises: i) the lipidoid in an amount from about 40 mol % to about 55 mol %; ii) the phospholipid in an amount from about 1.0 mol % to about 3 mol %; iii) the sterol in an amount from about 35 mol % to about 50 mol %; and iv) the PEGylated lipid in an amount from about 0.5 mol % to about 3 mol %.

In certain aspects, the lipid nanoparticle comprises: i) the lipidoid in an amount from about 45 mol % to about 55 mol %; ii) the phospholipid in an amount from about 1.0 mol % to about 3.0 mol %; iii) the sterol in an amount from about 40 mol % to about 50 mol %; and iv) the PEGylated lipid in an amount from about 1.0 mol % to about 2.0 mol %.

In certain aspects, the lipid nanoparticle comprises: i) the lipidoid in an amount of about 51.5 mol %; ii) the phospholipid in an amount of about 2.7 mol %; iii) the sterol in an amount of about 44.5 mol %; and iv) the PEGylated lipid in an amount of about 1.3 mol %.

In further aspects, provided herein is a pharmaceutical composition, comprising a nanoparticle composition of the invention, and a pharmaceutically acceptable carrier.

Also provided are methods of delivering a therapeutic agent, comprising administering to a subject in need thereof an effective amount of the nanoparticle composition of the invention, wherein the nanoparticle composition further comprises a therapeutic agent.

Also provided herein are methods of vaccination, comprising administering to a subject in need thereof an effective amount of the nanoparticle composition of the invention, wherein the nanoparticle composition further comprises an antigen or an mRNA molecule that encodes an antigen.

### BRIEF DESCRIPTION OF THE DRAWINGS

FIG. 1 shows the hEPO expression levels for the LNP shown in Table 1 compared to a control LNP comprising SM-102.

FIG. 2 shows the hEPO expression levels for each of the LNPs shown in Table 2 compared to control LNPs comprising SM-102 and ALC-0315.

FIG. 3 shows the hEPO expression level for the Compound 100 LNP shown in Table 3 compared to a control LNP comprising SM-102.

### DETAILED DESCRIPTION

The present invention is based on the surprising discovery of a class of lipidoid compounds useful for forming lipid nanoparticles that can deliver therapeutic agents.

In certain aspects, provided herein is a lipidoid having the structure of formula (I):

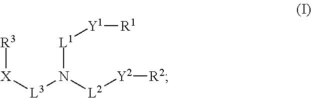

or a salt thereof;
wherein:
each of L¹, L², and L³ represents —(CH₂)ₘ—;
m is independently for each occurrence an integer selected from 1-20;
Y¹ is

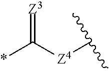

and * represents the connection to L¹;
Y² is

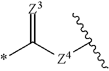

and * represents the connection to L²;
Z¹, Z², Z³, and Z⁴ are each independently O, S, or NH;
X is optionally substituted saturated or unsaturated cycloalkylene, heterocyclylene, heteroarylene, or arylene;
R¹ and R² are each independently optionally substituted linear (C₄-C₂₀)alkyl, branched (C₄-C₂₀)alkyl, linear (C₄-C₂₀)alkenyl, branched (C₄-C₂₀)alkenyl, linear (C₄-C₂₀)alkynyl, or branched (C₄-C₂₀)alkynyl, optionally wherein two substituents, together with the intervening atoms, form an aryl or 5-7 membered cycloalkyl ring; and
R³ is hydrogen, hydroxyl, hydroxyalkyl, amido, or an optionally substituted linear C₂-C₁₀ alkyl or branched C₃-C₁₀ alkyl.

In certain aspects, provided herein is a lipidoid having the structure of formula (I):

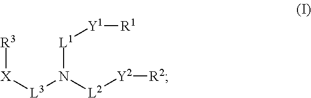

or a salt thereof;
wherein:
each of L¹, L², and L³ represents —(CH₂)ₘ—;
m independently for each occurrence is an integer selected from 1-20;
Y¹ is

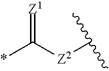

and * represents the connection to L¹;
Y² is

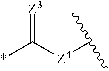

and * represents the connection to L²;
Z¹, Z², Z³, and Z⁴ are each independently O, S, or NH;
X is optionally substituted saturated or unsaturated cycloalkylene, heterocyclylene, heteroarylene, or arylene;
R¹ and R² are each independently optionally substituted linear (C₄-C₂₀)alkyl, branched (C₄-C₂₀)alkyl, linear (C₄-C₂₀)alkenyl, branched (C₄-C₂₀)alkenyl, linear (C₄-C₂₀)alkynyl, or branched (C₄-C₂₀)alkynyl; and
R³ is hydrogen, hydroxyl, hydroxyalkyl, amido, or an optionally substituted linear C₂-C₁₀ alkyl or branched C₃-C₁₀ alkyl.

In certain aspects, at least one of Y¹ or Y² is *—(C═O)O—. In certain aspects, Y¹ is *—(C═O)O—. In certain aspects, Y² is *—(C═O)O—.

In certain aspects, Y¹ and Y² are different. In certain other aspects, Y¹ and Y² are the same.

In certain aspects, the lipidoid has the structure of formula (II):

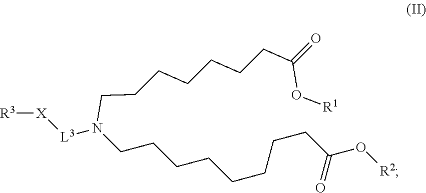

or a salt thereof;
wherein:
L³ is —(CH₂)ₘ—;
m is an integer selected from 1-10;
X is an optionally substituted saturated or unsaturated cylcoalkylene, heterocyclylene, heteroarylene, or arylene;
R¹ and R² are each independently optionally substituted linear (C₄-C₂₀)alkyl, branched (C₄-C₂₀)alkyl, linear (C₄-C₂₀)alkenyl, branched (C₄-C₂₀)alkenyl, linear (C₄-C₂₀)alkynyl or branched (C₄-C₂₀)alkynyl, optionally wherein two substituents, together with the intervening atoms, form an aryl or 5-7 membered cycloalkyl ring; and
R³ is hydrogen, hydroxyl, hydroxyalkyl, amido, or an optionally substituted linear C₂-C₁₀ alkyl or branched C₃-C₁₀ alkyl.

In certain aspects, the lipidoid has the structure of formula (II):

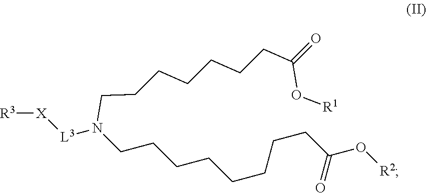

or a salt thereof;
wherein:
L³ is —(CH₂)ₘ—;
m is an integer selected from 1-10;
X is an optionally substituted saturated or unsaturated cylcoalkylene, heterocyclylene, heteroarylene, or arylene;
R¹ and R² are each independently optionally substituted linear (C₄-C₂₀)alkyl, branched (C₄-C₂₀)alkyl, linear (C₄-C₂₀)alkenyl, branched (C₄-C₂₀)alkenyl, linear (C₄-C₂₀)alkynyl or branched (C₄-C₂₀)alkynyl; and
R³ is hydrogen, hydroxyl, hydroxyalkyl, amido, or an optionally substituted linear C₂-C₁₀ alkyl or branched C₃-C₁₀ alkyl.

In certain aspects, the lipidoid has the structure of formula (III):

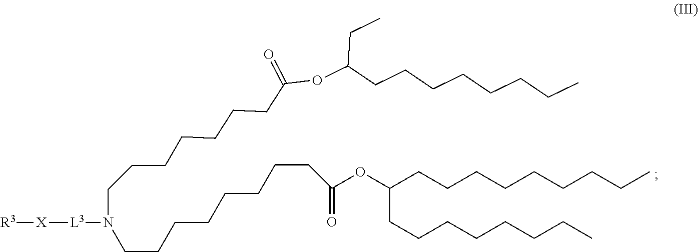

or a salt thereof;
wherein:
L³ is —(CH₂)ₘ—;
m is an integer selected from 1-10;
X is an optionally substituted saturated or unsaturated cycloalkylene, heterocyclylene, heteroarylene, or arylene; and
R³ is hydrogen, hydroxyl, hydroxyalkyl, amido, or an optionally substituted linear C₂-C₁₀ alkyl or branched C₃-C₁₀ alkyl.

In certain aspects, the lipidoid has the structure of formula (IV):

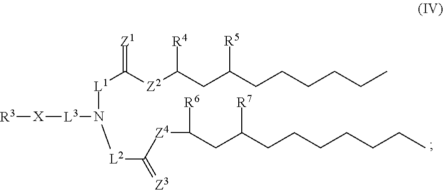

or a salt thereof;
wherein:
each of L¹, L², and L³ represent —(CH₂)ₘ—;
m is an integer selected from 1-20;
Z¹, Z², Z³, and Z⁴ are each independently O, S, or NH;
X is an optionally substituted saturated or unsaturated cycloalkylene, heterocyclylene, heteroarylene, or arylene;
R³ is hydrogen, hydroxyl, hydroxyalkyl, amido, or an optionally substituted linear C₂-C₁₀ alkyl or branched C₃-C₁₀ alkyl.
R⁴ and R⁶ are each independently H, linear C₂-C₁₀ alkyl or branched C₃-C₁₀ alkyl;
R⁵ is H, linear C₂-C₁₀ alkyl, or branched C₃-C₁₀ alkyl,
or R⁵ and R⁴, taken together with the intervening atoms, form an aryl or a 5- to 7-membered cycloalkyl; and
R⁷ is H, linear C₂-C₁₀ alkyl, or branched C₃-C₁₀ alkyl,
or R⁷ and R⁶, taken together with the intervening atoms, form an aryl or a 5- to 7-membered cycloalkyl.

In certain aspects, the lipidoid has the structure of formula (IV):

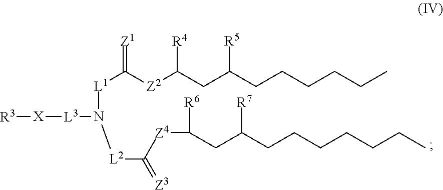

or a salt thereof;
wherein:
each of L¹, L², and L³ represent —(CH₂)ₘ—;
m is independently for each occurrence an integer selected from 1-20;
Z¹, Z², Z³, and Z⁴ are each independently O, S, or NH;
X is an optionally substituted saturated or unsaturated cycloalkylene, heterocyclylene, heteroarylene, or arylene;
R³ is hydrogen, hydroxyl, hydroxyalkyl, amido, or an optionally substituted linear or branched C₃-C₁₀ alkyl.
R⁴ and R⁶ are each independently H, linear C₂-C₁₀ alkyl or branched C₃-C₁₀ alkyl;
R⁵ is H, linear C₂-C₁₀ alkyl, or branched C₃-C₁₀ alkyl; and
R⁷ is H, linear C₂-C₁₀ alkyl, or branched C₃-C₁₀ alkyl.

In certain aspects, the lipidoid has the structure of formula (V):

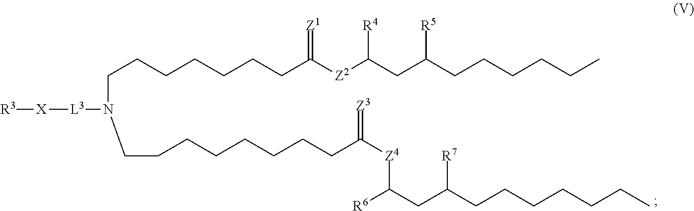

or a salt thereof;
wherein:
L³ represents —(CH₂)ₘ—;
m is an integer selected from 1-10;
each of Z¹, Z², Z³, and Z⁴ are independently O, S, or NH;
X is an optionally substituted saturated or unsaturated cycloalkylene, heterocyclylene, heteroarylene, or arylene;
R³ is hydrogen, hydroxyl, hydroxyalkyl, amido, or an optionally substituted linear C₂-C₁₀ alkyl or branched C₃-C₁₀ alkyl.
R⁴ and R⁶ are each independently H, linear C₂-C₁₀ alkyl or branched C₃-C₁₀ alkyl;
R⁵ is H, linear C₂-C₁₀ alkyl, or branched C₃-C₁₀ alkyl,
or R⁵ and R⁴, taken together with the intervening atoms, form an aryl or a 5- to 7-membered cycloalkyl; and
R⁷ is H, linear C₂-C₁₀ alkyl, or branched C₃-C₁₀ alkyl,
or R⁷ and R⁶, taken together with the intervening atoms, form an aryl or a 5- to 7-membered cycloalkyl.

In certain aspects, the lipidoid has the structure of formula (V):

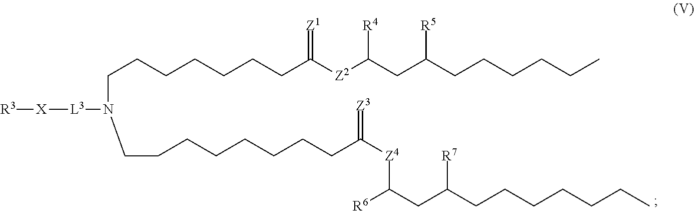

or a salt thereof;
wherein:
L³ represents —(CH₂)ₘ—;
m is an integer selected from 1-10;
each of Z¹, Z², Z³, and Z⁴ are independently O, S, or NH;
X is an optionally substituted saturated or unsaturated cycloalkylene, heterocyclylene, heteroarylene, or arylene;
R³ is hydrogen, hydroxyl, hydroxyalkyl, amido, or an optionally substituted linear C₂-C₁₀ alkyl or branched C₃-C₁₀ alkyl.
R⁴ and R⁶ are each independently H, linear C₂-C₁₀ alkyl or branched C₃-C₁₀ alkyl;
R⁵ is H, linear C₂-C₁₀ alkyl, or branched C₃-C₁₀ alkyl; and
R⁷ is H, linear C₂-C₁₀ alkyl, or branched C₃-C₁₀ alkyl.

In certain aspects, X is a five-membered saturated or unsaturated heterocyclyl.

In further aspects, X represents a saturated or partially unsaturated heterocyclyl comprising two heteroatoms.

In certain aspects, X represents a saturated or partially unsaturated heterocyclyl substituted with oxo (═O).

In further aspects, X is

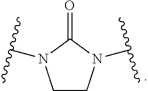 [SMILES: *N1CCN(*)C1=O]

In certain aspects, X comprises a five-membered heteroarylene.

In certain aspects, X comprises a six-membered arylene.

In certain aspects, X comprises a four to seven-membered heterocyclylene.

In certain aspects, X comprises a four to seven-membered cycloalkylene.

In certain aspects, —X—R³ is selected from:

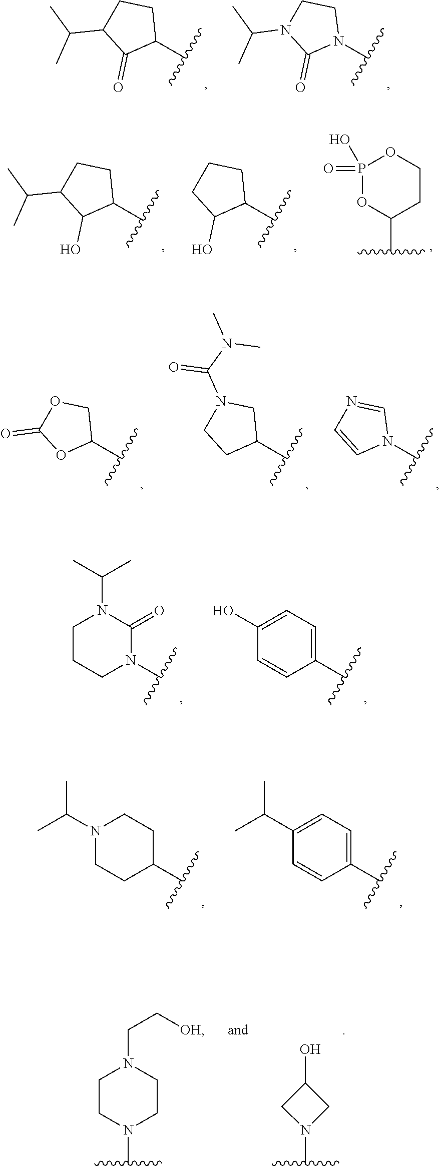

In certain aspects, L³ is —(CH₂)₃—.

In certain aspects, the lipidoid has the structure of formula (VI);

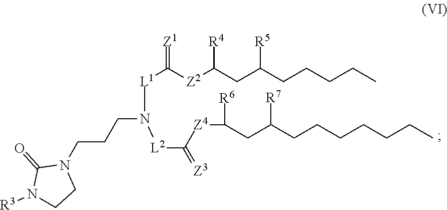

or a salt thereof;
wherein:
each of L¹ and L² represent —(CH₂)ₘ—;
m is independently for each occurrence an integer selected from 1-20;
each of Z¹, Z², Z³, and Z⁴ are independently O, S, or NH;
R³ is hydrogen, hydroxyl, hydroxyalkyl, amido, or an optionally substituted linear C₂-C₁₀ alkyl or branched C₃-C₁₀ alkyl.
R⁴ and R⁶ are each independently H, linear C₂-C₁₀ alkyl or branched C₃-C₁₀ alkyl;
R⁵ is H, linear C₂-C₁₀ alkyl, or branched C₃-C₁₀ alkyl,
or R⁵ and R⁴, taken together with the intervening atoms, form an aryl or a 5- to 7-membered cycloalkyl; and
R⁷ is H, linear C₂-C₁₀ alkyl, or branched C₃-C₁₀ alkyl,
or R⁷ and R⁶, taken together with the intervening atoms, form an aryl or a 5- to 7-membered cycloalkyl.

In certain aspects, the lipidoid has the structure of formula (VI);

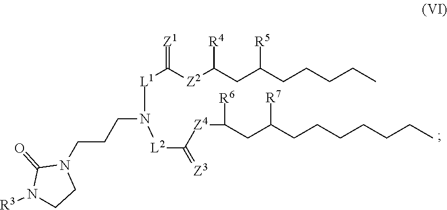

or a salt thereof;
wherein:
each of L¹ and L² represent —(CH₂)ₘ—;
m is independently for each occurrence an integer selected from 1-20;
each of Z¹, Z², Z³, and Z⁴ are independently O, S, or NH;
R³ is hydrogen, hydroxyl, hydroxyalkyl, amido, or an optionally substituted linear or branched C₃-C₁₀ alkyl.
R⁴ and R⁶ are each independently H, linear C₂-C₁₀ alkyl or branched C₃-C₁₀ alkyl;
R⁵ is H, linear C₂-C₁₀ alkyl, or branched C₃-C₁₀ alkyl; and
R⁷ is H, linear C₂-C₁₀ alkyl, or branched C₃-C₁₀ alkyl.

In certain aspects, m is independently for each occurrence an integer selected from 2-10.

In certain aspects, at least one of L¹ and L² is —(CH₂)₇—.

In certain aspects, at least one of L¹ and L² is —(CH₂)₈—.

In certain aspects, L¹ and L² are the same.

In certain aspects, L¹ and L² are different.

In certain aspects, the lipidoid has the structure of formula (VII);

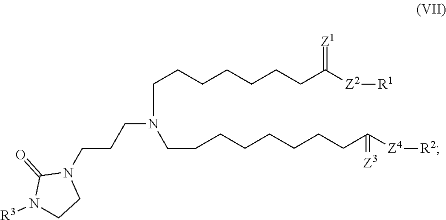

or a salt thereof;
wherein:
R³ is hydrogen, hydroxyalkyl, amido, or an optionally substituted linear C₂-C₁₀ alkyl or branched C₃-C₁₀ alkyl;
each of Z¹, Z², Z³, and Z⁴ are independently O, S, or NH;
R¹ and R² are each independently optionally substituted linear (C₄-C₂₀)alkyl, branched (C₄-C₂₀)alkyl, linear (C₄-C₂₀)alkenyl, branched (C₄-C₂₀)alkenyl, linear (C₄-C₂₀)alkynyl, or branched (C₄-C₂₀)alkynyl, optionally wherein two substituents, together with the intervening atoms, form an aryl or 5-7 membered cycloalkyl ring.

In certain aspects, the lipidoid has the structure of formula (VII);

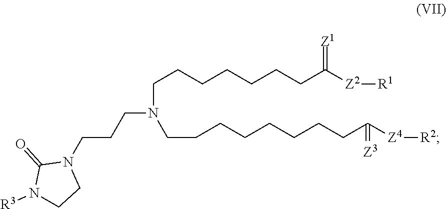

or a salt thereof;
wherein:
R³ is hydrogen, hydroxyalkyl, amido, or an optionally substituted linear C₂-C₁₀ alkyl or branched C₃-C₁₀ alkyl;
each of Z¹, Z², Z³, and Z⁴ are independently O, S, or NH;
R¹ and R² are each independently optionally substituted linear (C₄-C₂₀)alkyl, branched (C₄-C₂₀)alkyl, linear (C₄-C₂₀)alkenyl, branched (C₄-C₂₀)alkenyl, linear (C₄-C₂₀)alkynyl, or branched (C₄-C₂₀)alkynyl.

In certain aspects, R³ is isopropyl.

In certain aspects, R¹ and R² are the same.

In certain aspects, R¹ and R² are different.

In certain aspects, R¹ and R² are each independently branched (C₁₁-C₂₀)alkyl.

In certain aspects, R¹ is

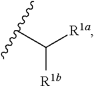

and R^{1a} and R^{1b} are each independently (C₂-C₁₈)alkyl, wherein R¹ contains from 8 to 20 total carbon atoms.

In certain aspects, R^{1a} and R^{1b} are the same.

In certain aspects, R^{1a} and R^{1b} are different.

In certain aspects, R² is

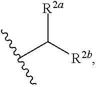

and R^{2a} and R^{2b} are each independently (C₂-C₁₈)alkyl, wherein R² contains from 8 to 20 total carbon atoms.

In certain aspects, R^{2a} and R^{2b} are the same.

In certain aspects, R^{2a} and R^{2b} are different.

In certain aspects, R¹ is:

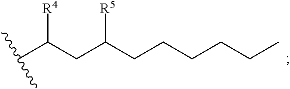

and
R⁴ and R⁵ are each independently H, C₂-C₁₀ alkyl, or R⁴ and R⁵, taken together with the intervening atoms, form an aryl or a 5- to 7-membered cycloalkyl.

In certain aspects, R¹ is:

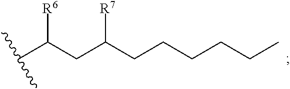

and
R⁴ and R⁵ are each independently H or C₂-C₁₀ alkyl.

In certain aspects, R² is:

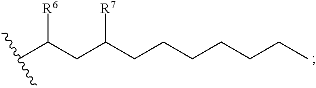

and
R⁶ and R⁷ are each independently H, C₂-C₁₀ alkyl, or R⁶ and R⁷, taken together with the intervening atoms, form an aryl or a 5- to 7-membered cycloalkyl.

In certain aspects, R² is:

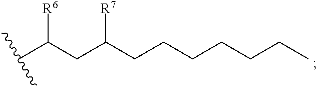

and
R⁶ and R⁷ are each independently H or C₂-C₁₀ alkyl.

In certain aspects, the lipidoid has the structure of formula (VIII):

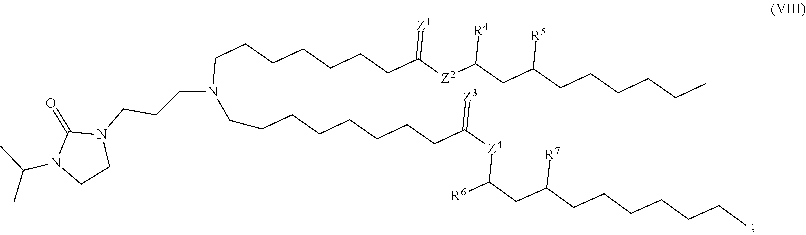

or a salt thereof;
wherein:
each of Z¹, Z², Z³, and Z⁴ are independently O, S, or NH;
R⁴ and R⁶ are each independently H, linear C₂-C₁₀ alkyl or branched C₃-C₁₀ alkyl;
R⁵ is H, linear C₂-C₁₀ alkyl, or branched C₃-C₁₀ alkyl,
or R⁵ and R⁴, taken together with the intervening atoms, form an aryl or a 5- to 7-membered cycloalkyl; and
R⁷ is H, linear C₂-C₁₀ alkyl, or branched C₃-C₁₀ alkyl,
or R⁷ and R⁶, taken together with the intervening atoms, form an aryl or a 5- to 7-membered cycloalkyl.

In certain aspects, the lipidoid has the structure of formula (VIII):

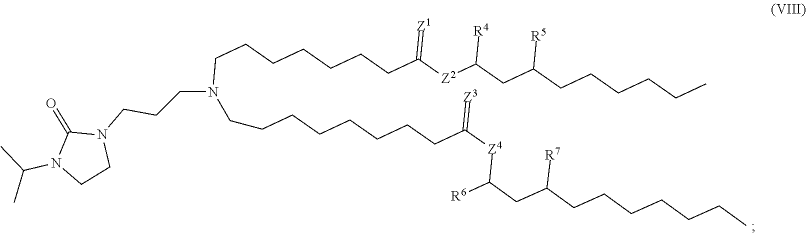

or a salt thereof;
wherein:
each of Z¹, Z², Z³, and Z⁴ are independently O, S, or NH;
R⁴ and R⁶ are each independently H, linear C₂-C₁₀ alkyl or branched C₃-C₁₀ alkyl;
R⁵ is H, linear C₂-C₁₀ alkyl, or branched C₃-C₁₀ alkyl; and
R⁷ is H, linear C₂-C₁₀ alkyl, or branched C₃-C₁₀ alkyl.

In certain aspects, Z¹, Z², Z³, and Z⁴ are each O.

In certain exemplary embodiments, the lipidoid is:

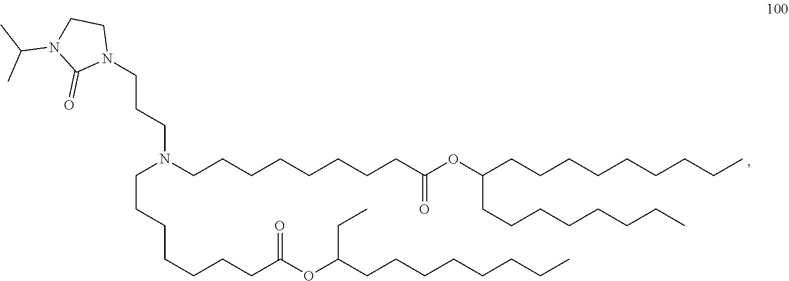 [SMILES: CCCCCCCCCC(CCCCCCCC)OC(=O)CCCCCCCCN(CCCCCCCC(=O)OC(CC)CCCCCCCC)CCCN1CCN(C(C)C)C1=O]

or a salt thereof.

In certain exemplary embodiments, the lipidoid is selected from:

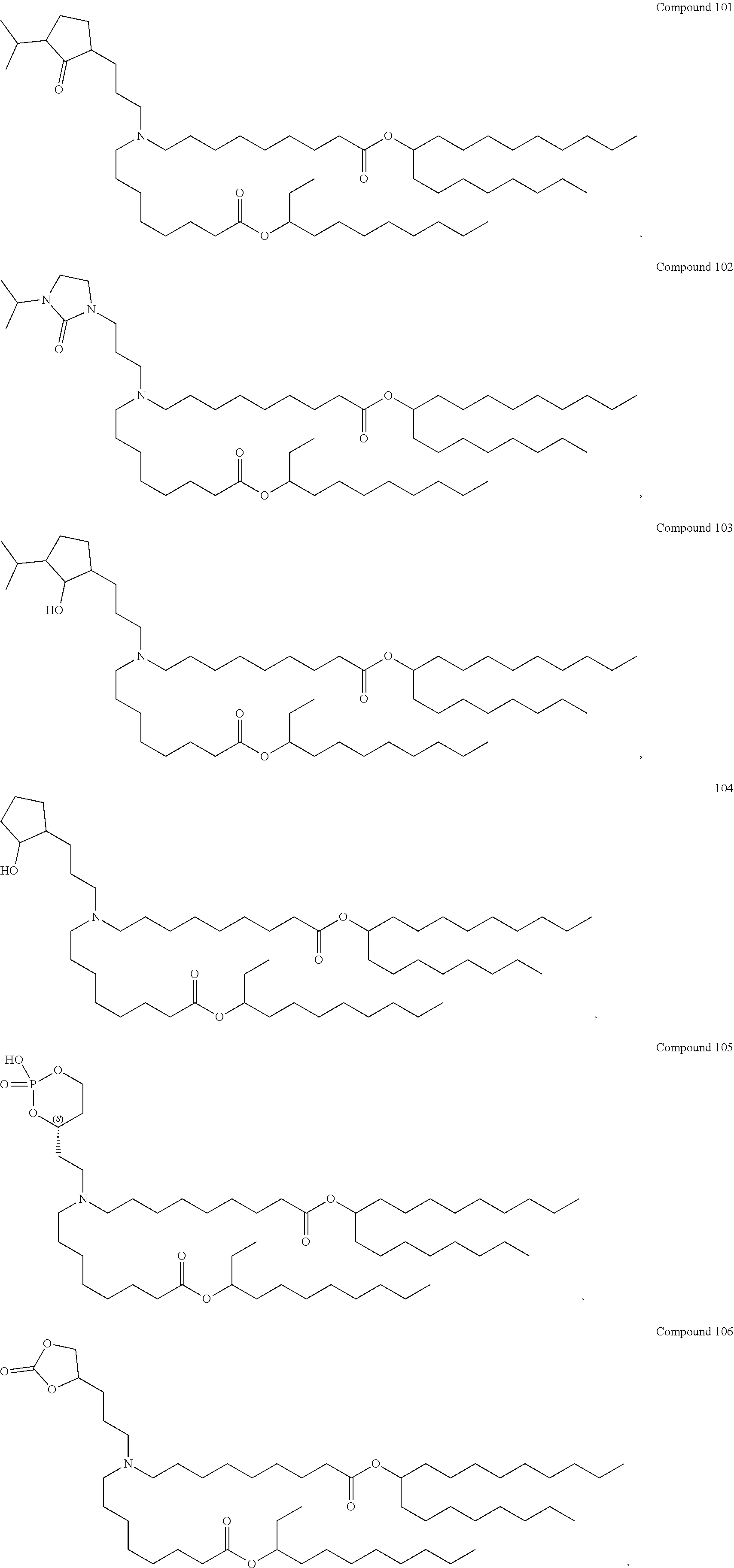

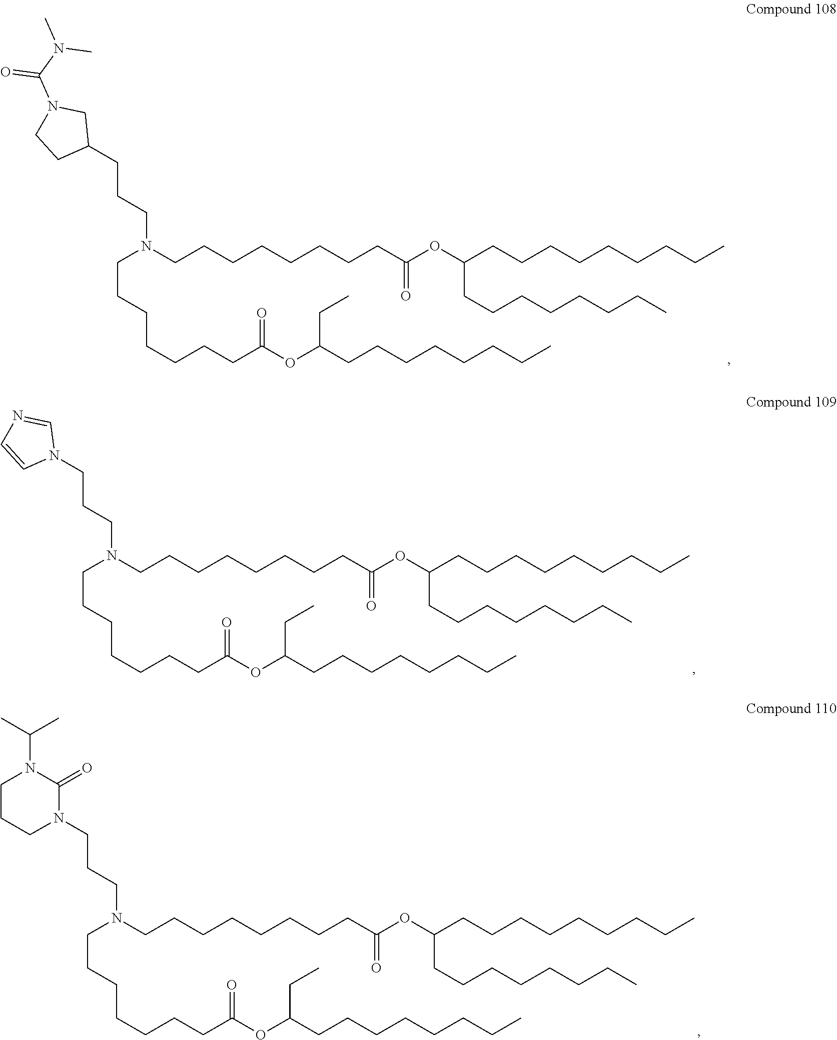 [SMILES: CCCCCCCCCC(CCCCCCCC)OC(=O)CCCCCCCCN(CCCCCCCC(=O)OC(CC)CCCCCCCC)CCCC1CCN(C(=O)N(C)C)C1 ; CCCCCCCCCC(CCCCCCCC)OC(=O)CCCCCCCCN(CCCCCCCC(=O)OC(CC)CCCCCCCC)CCCn1ccnc1 ; CCCCCCCCCC(CCCCCCCC)OC(=O)CCCCCCCCN(CCCCCCCC(=O)OC(CC)CCCCCCCC)CCCN1CCCN(C(C)C)C1=O]

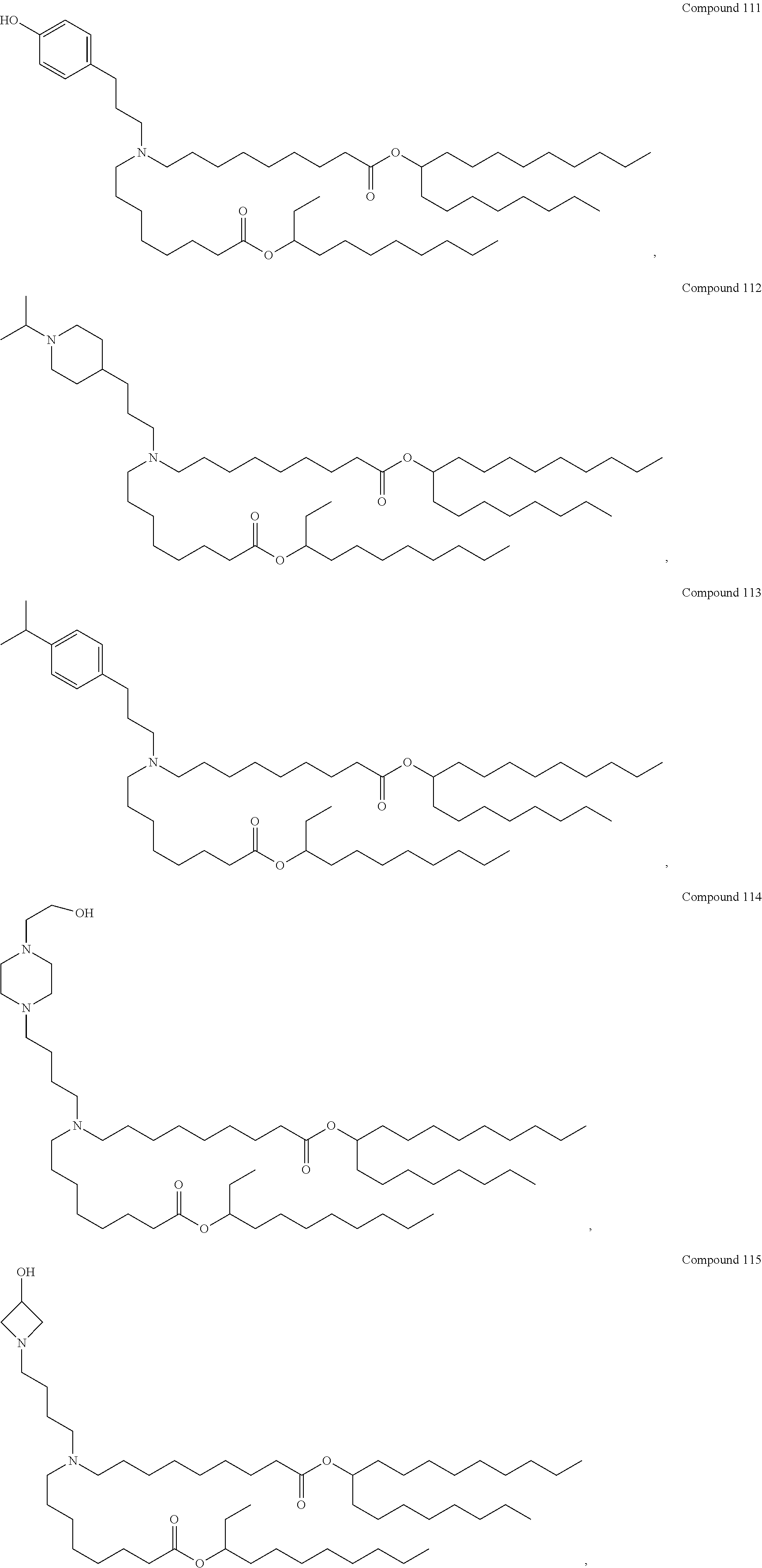

or a salt thereof.

In certain aspects, provided herein is a lipidoid having the structure of formula (I-P):

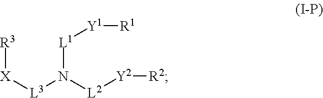

or a salt thereof;
wherein:
each of L¹, L², and L³ represents —(CH₂)ₘ—;
m is independently for each occurrence an integer selected from 1-20;
Y¹ is

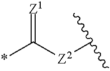

and * represents the connection to L¹;
Y² is

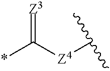

and * represents the connection to L²;
Z¹, Z², Z³, and Z⁴ are each independently O, S, or NH;
X is optionally substituted saturated or unsaturated heterocyclyl;
R¹ and R² are each independently optionally substituted linear (C₄-C₂₀)alkyl, branched (C₄-C₂₀)alkyl, linear (C₄-C₂₀)alkenyl, branched (C₄-C₂₀)alkenyl, linear (C₄-C₂₀)alkynyl, or branched (C₄-C₂₀)alkynyl, optionally wherein two substituents, together with the intervening atoms, form an aryl or 5-7 membered cycloalkyl ring; and
R³ is an optionally substituted linear or branched C₂-C₁₀ alkyl.

In certain aspects, the lipidoid has the structure of formula (II-P):

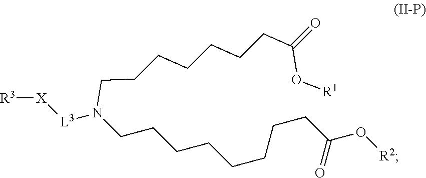

or a salt thereof;
wherein:
L³ is —(CH₂)ₘ—;
m is independently for each occurrence an integer selected from 1-10;
X is an optionally substituted saturated or unsaturated heterocyclyl;
R¹ and R² are each independently optionally substituted linear (C₄-C₂₀)alkyl, branched (C₄-C₂₀)alkyl, linear (C₄-C₂₀)alkenyl, branched (C₄-C₂₀)alkenyl, linear (C₄-C₂₀)alkynyl or branched (C₄-C₂₀)alkynyl, optionally wherein two substituents, together with the intervening atoms, form an aryl or 5-7 membered cycloalkyl ring; and
R³ is an optionally substituted linear or branched C₂-C₁₀ alkyl.

In certain aspects, the lipidoid has the structure of formula (III-P):

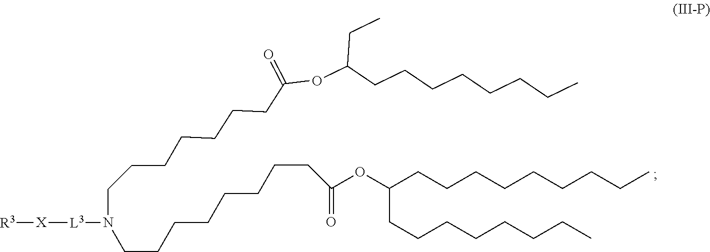

or a salt thereof;
wherein:
L³ is —(CH₂)ₘ—;
m is independently for each occurrence an integer selected from 1-10;
X is an optionally substituted saturated or unsaturated heterocyclyl; and
R³ is an optionally substituted linear or branched C₂-C₁₀ alkyl.

In certain aspects, the lipidoid has the structure of formula (IV-P):

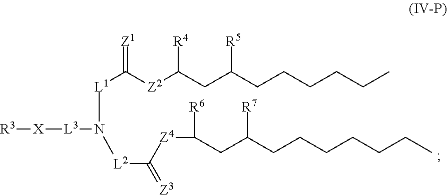

or a salt thereof;
wherein:
each of L¹, L², and L³ represent —(CH₂)ₘ—;
m is independently for each occurrence an integer selected from 1-20;
Z¹, Z², Z³, and Z⁴ are each independently O, S, or NH;
X is an optionally substituted saturated or unsaturated heterocyclyl;
R³ is an optionally substituted linear or branched C₂-C₁₀ alkyl;
R⁴ and R⁶ are each independently H, linear C₂-C₁₀ alkyl or branched C₂-C₁₀ alkyl;
R⁵ is H, linear C₂-C₁₀ alkyl, or branched C₂-C₁₀ alkyl,
or R⁵ and R⁴, taken together with the intervening atoms, form an aryl or a 5- to 7-membered cycloalkyl; and
R⁷ is H, linear C₂-C₁₀ alkyl, or branched C₂-C₁₀ alkyl,
or R⁷ and R⁶, taken together with the intervening atoms, form an aryl or a 5- to 7-membered cycloalkyl.

In certain aspects, the lipidoid has the structure of formula (V-P):

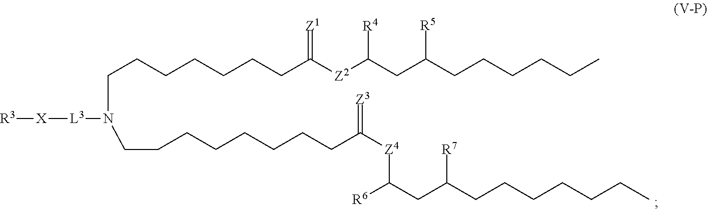

or a salt thereof;
wherein:
L³ represents —(CH₂)ₘ—;
m is independently for each occurrence an integer selected from 1-10;
each of Z¹, Z², Z³, and Z⁴ are independently O, S, or NH;
X is an optionally substituted saturated or unsaturated heterocyclyl;
R³ is an optionally substituted linear or branched C₂-C₁₀ alkyl;
R⁴ and R⁶ are each independently H, linear C₂-C₁₀ alkyl or branched C₂-C₁₀ alkyl;
R⁵ is H, linear C₂-C₁₀ alkyl, or branched C₂-C₁₀ alkyl,
or R⁵ and R⁴, taken together with the intervening atoms, form an aryl or a 5- to 7-membered cycloalkyl; and
R⁷ is H, linear C₂-C₁₀ alkyl, or branched C₂-C₁₀ alkyl,
or R⁷ and R⁶, taken together with the intervening atoms, form an aryl or a 5- to 7-membered cycloalkyl.

In certain aspects, the lipidoid has the structure of formula (VI-P);

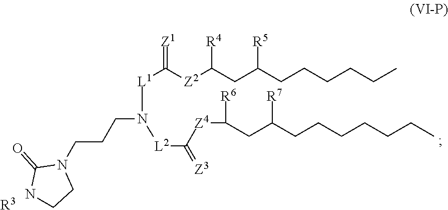

or a salt thereof;
wherein:
each of L^{t} and L² represent —(CH₂)ₘ—;
m is independently for each occurrence an integer selected from 1-20;
each of Z¹, Z², Z³, and Z⁴ are independently O, S, or NH;
R³ is an optionally substituted linear or branched C₂-C₁₀ alkyl;
R⁴ and R⁶ are each independently H, linear C₂-C₁₀ alkyl or branched C₂-C₁₀ alkyl;
R⁵ is H, linear C₂-C₁₀ alkyl, or branched C₂-C₁₀ alkyl,
or R⁵ and R⁴, taken together with the intervening atoms, form an aryl or a 5- to 7-membered cycloalkyl; and
R⁷ is H, linear C₂-C₁₀ alkyl, or branched C₂-C₁₀ alkyl,
or R⁷ and R⁶, taken together with the intervening atoms, form an aryl or a 5- to 7-membered cycloalkyl.

In certain aspects, the lipidoid has the structure of formula (VII-P);

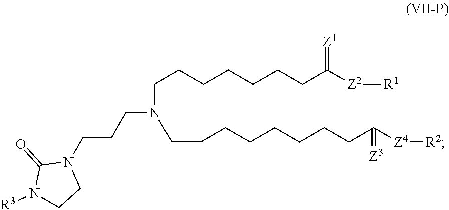

or a salt thereof;
wherein:
R³ is optionally substituted linear or branched C₂-C₁₀ alkyl;
each of Z¹, Z², Z³, and Z⁴ are independently O, S, or NH;
R¹ and R² are each independently optionally substituted linear (C₄-C₂₀)alkyl, branched (C₄-C₂₀)alkyl, linear (C₄-C₂₀)alkenyl, branched (C₄-C₂₀)alkenyl, linear (C₄-C₂₀)alkynyl, or branched (C₄-C₂₀)alkynyl, optionally wherein two substituents, together with the intervening atoms, form an aryl or 5-7 membered cycloalkyl ring.

In certain aspects, the lipidoid has the structure of formula (VIII-P):

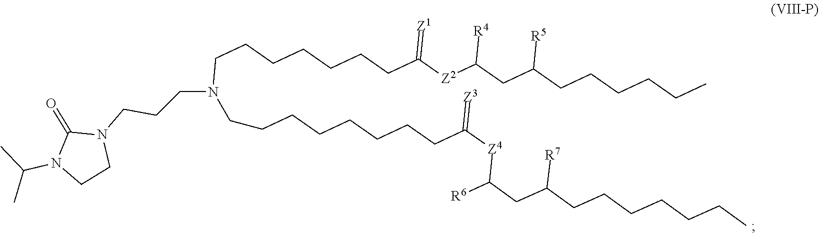

or a salt thereof;
wherein:
each of Z¹, Z², Z³, and Z⁴ are independently O, S, or NH;
R⁴ and R⁶ are each independently H, linear C₂-C₁₀ alkyl or branched C₂-C₁₀ alkyl;
R⁵ is H, linear C₂-C₁₀ alkyl, or branched C₂-C₁₀ alkyl,
   or R⁵ and R⁴, taken together with the intervening atoms, form an aryl or a 5- to 7-membered cycloalkyl; and
R⁷ is H, linear C₂-C₁₀ alkyl, or branched C₂-C₁₀ alkyl,
   or R⁷ and R⁶, taken together with the intervening atoms, form an aryl or a 5- to 7-membered cycloalkyl.

In any of the preceding embodiments, the salt of the lipidoid may be a pharmaceutically acceptable salt.

### Nanoparticle Compositions/Lipid Nanoformulations/Lipid-Based Carriers

In certain embodiments, provided herein is a nanoparticle composition comprising a lipidoid of the invention, or a pharmaceutically acceptable salt thereof. In certain embodiments, the nanoparticle composition comprises a plurality of lipidoids of the invention, or a pharmaceutically acceptable salt thereof.

As used here, “nanoparticle composition” is used interchangeably with the terms “lipid-based carrier,” “lipid nanoformulation,” and “lipid nanoparticle.”

In certain embodiments, the nanoparticle composition further comprises a lipid. In certain such embodiments, the lipid is a cationic, anionic, ionizable, or zwitterionic lipid.

In some embodiments, compounds described herein are formulated into a lipid-based carrier (or lipid nanoformulation). In some embodiments, the lipid-based carrier (or lipid nanoformulation) is a liposome or a lipid nanoparticle (LNP). In some embodiments, the lipid-based carrier is an LNP.

In some embodiments, the lipid-based carrier (or lipid nanoformulation) comprises a cationic lipid (e.g., an ionizable lipid such as a lipidoid described herein), a non-cationic lipid (e.g., phospholipid), a structural lipid (e.g., cholesterol), and a PEG-modified lipid. In some embodiments, the lipid-based carrier (or lipid nanoformulation) contains one or more compounds described herein, or a pharmaceutically acceptable salt thereof.

All above descriptions and all embodiments discussed in the above aspects relating to the aspects of the lipid compounds, including the compounds covered by formula (I), and the exemplary formulas for lipids having ionizable head groups are all applicable to these aspects of the invention relating to the lipid-based carriers (or a lipid nanoformulation).

As described herein, suitable compounds to be used in the lipid-based carrier (or lipid nanoformulation) include all the isomers and isotopes of the compounds described above, as well as all the pharmaceutically acceptable salts, solvates, or hydrates thereof, and all crystal forms, crystal form mixtures, and anhydrides or hydrates.

In addition to one or more compounds described herein, the lipid-based carrier (or lipid nanoformulation) may further include a second lipid. In some embodiments, the second lipid is a cationic lipid, a non-cationic (e.g., neutral, anionic, or zwitterionic) lipid, or an ionizable lipid.

One or more naturally occurring and/or synthetic lipid compounds may be used in the preparation of the lipid-based carrier (or lipid nanoformulation).

The lipid-based carrier (or lipid nanoformulation) may contain positively charged (cationic) lipids, neutral lipids, negatively charged (anionic) lipids, or a combination thereof.

In some embodiments, the lipid nanoparticle of the invention may be conjugated to a targeting moiety (e.g., an antibody or antigen-binding fragment thereof) through a linking group. Various linking groups known in the art may be used in the lipid nanoparticles of the invention, and can comprise one or more of optionally substituted alkylene, optionally substituted heteroalkylene, optionally substituted alkenylene, optionally substituted heteroalkenylene, optionally substituted alkynylene, optionally substituted heteroalkynylene, optionally substituted cycloalkylene, optionally substituted heterocycloalkylene, optionally substituted arylene, optionally substituted heteroarylene, a peptide moiety, a dipeptide moiety, —(C═O)—, a disulfide, a hydrazone, thioester, sulfone, sulfoxide, thiosulfinate, thiosulfonate, sulfate, sulfonate, sulfonylurea, ether, thioether, ester, amide, carbonate, carbamate, urea, sulfamide, succinimide, maleimide, phosphate, diphosphate, triazole, or a saccharide, or a combination thereof. Suitable linking groups are described, e.g., in WO 2024/015229, WO 2024/006272, and WO 2023/225359.

### Cationic Lipids (Positively Charged) and Ionizable Lipids

In some embodiments, the lipid-based carrier (or lipid nanoformulation) comprises one or more cationic lipids, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions.

Exemplary cationic lipids include one or more amine group(s) which bear the positive charge. Examples of positively charged (cationic) lipids include, but are not limited to, N,N′-dimethyl-N,N′-dioctacyl ammonium bromide (DDAB) and chloride DDAC), N-(1-(2,3-dioleyloxy)propyl)-N,N,N-trimethylammonium chloride (DOTMA), 3β-[N—(N′,N′-dimethylaminoethyl)carbamoyl) cholesterol (DC-chol), 1,2-dioleoyloxy-3-[trimethylammonio]-propane (DOTAP), 1,2-dioctadecyloxy-3-[trimethylammonio]-propane (DSTAP), and 1,2-dioleoyloxypropyl-3-dimethyl-hydroxy ethyl ammonium chloride (DORI), N,N-dioleyl-N,N-dimethylammonium chloride (DODAC), N,N-dimethyl-2,3-dioleyloxy)propylamine (DODMA), 1,2-Dioleoyl-3-Dimethylammonium-propane (DODAP), 1,2-Dioleoylcarbamyl-3-Dimethylammonium-propane (DOCDAP), 1,2-Dilineoyl-3-Dimethylammonium-propane (DLINDAP), 3-Dimethylamino-2-(Cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis, cis-9′,12′-octadecadienoxy)propane (CpLin DMA), N,N-Dimethyl-3,4-dioleyloxybenzylamine (DMOBA), and the cationic lipids described in e.g. Martin et al., Current Pharmaceutical Design, pages 1-394, which is herein incorporated by reference in its entirety. In some embodiments, the lipid-based carrier (or lipid nanoformulation) comprises more than one cationic lipid.

In some embodiments, the lipid-based carrier (or lipid nanoformulation) comprises a cationic lipid having an effective pKa over 6.0. In some embodiments, the lipid-based carrier (or lipid nanoformulation) further comprises a second cationic lipid having a different effective pKa (e.g., greater than the first effective pKa) than the first cationic lipid.

In some embodiments, cationic lipids that can be used in the lipid-based carrier (or lipid nanoformulation) include, for example those described in Table 4 of WO 2019/217941, which is incorporated by reference.

In some embodiments, the cationic lipid is an ionizable lipid (e.g., a lipid that is protonated at low pH, but that remains neutral at physiological pH). In some embodiments, the lipid-based carrier (or lipid nanoformulation) may comprise one or more additional ionizable lipids, different than the ionizable lipids described herein. Exemplary ionizable lipids include, but are not limited to,

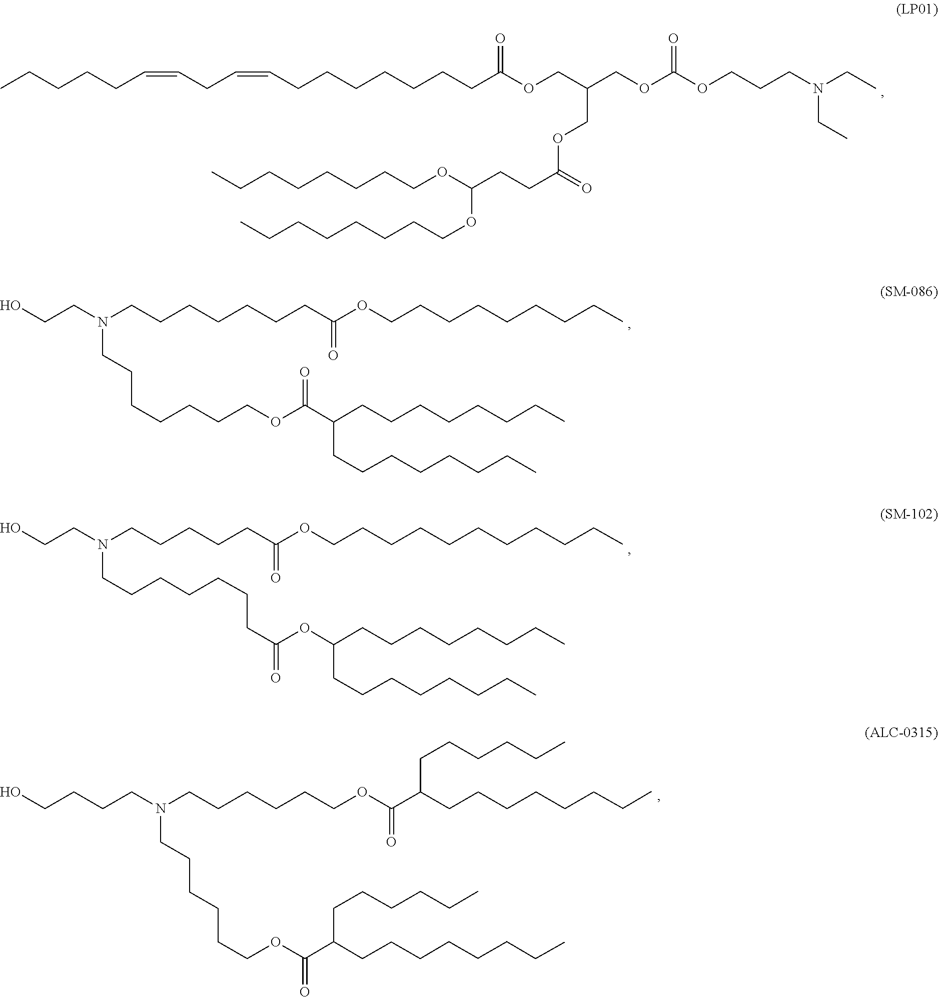 [SMILES: CCCCC/C=C\C/C=C\CCCCCCCC(=O)OCC(COC(=O)CCC(OCCCCCCCC)OCCCCCCCC)COC(=O)OCCCN(CC)CC ; CCCCCCCCCOC(=O)CCCCCCCN(CCO)CCCCCCCOC(=O)C(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CCCCCC)C(=O)OCCCCCCN(CCCCO)CCCCCCOC(=O)C(CCCCCC)CCCCCCCC]

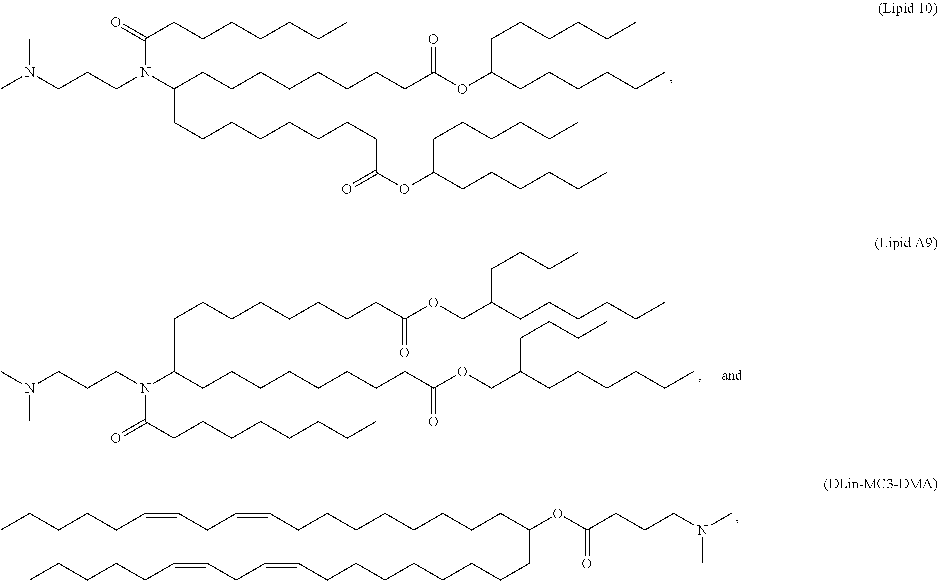

(see WO 2017/004143A1, which is incorporated herein by reference in its entirety).

In some embodiments, the lipid-based carrier (or lipid nanoformulation) further comprises one or more compounds described by WO 2021/113777 (e.g., a lipid of Formula (3) such as a lipid of Table 3 of WO 2021/113777), which is incorporated herein by reference in its entirety.

In some embodiments, the ionizable lipid is a lipid disclosed in Hou, X., et al. Nat Rev Mater 6, 1078-1094 (2021). https://doi.org/10.1038/s41578-021-00358-0 (e.g., L319, C12-200, and DLin-MC3-DMA), (which is incorporated by reference herein in its entirety).

Examples of other ionizable lipids that can be used in lipid-based carrier (or lipid nanoformulation) include, without limitation, one or more of the following formulas: X of US 2016/0311759; I of US 20150376115 or in US 2016/0376224; Compound 5 or Compound 6 in US 2016/0376224; I, IA, or II of U.S. Pat. No. 9,867,888; I, II or III of US 2016/0151284; I, IA, II, or IIA of US 2017/0210967; I-c of US 2015/0140070; A of US 2013/0178541; I of US 2013/0303587 or US 2013/0123338; I of US 2015/0141678; II, III, IV, or V of US 2015/0239926; I of US 2017/0119904; I or II of WO 2017/117528; A of US 2012/0149894; A of US 2015/0057373; A of WO 2013/116126; A of US 2013/0090372; A of US 2013/0274523; A of US 2013/0274504; A of US 2013/0053572; A of WO 2013/016058; A of WO 2012/162210; I of US 2008/042973; I, II, III, or IV of US 2012/01287670; I or II of US 2014/0200257; I, II, or III of US 2015/0203446; I or III of US 2015/0005363; I, IA, IB, IC, ID, II, IIA, IIB, IIC, IID, or III-XXIV of US 2014/0308304; of US 2013/0338210; I, II, III, or IV of WO 2009/132131; A of US 2012/01011478; I or XXXV of US 2012/0027796; XIV or XVII of US 2012/0058144; of US 2013/0323269; I of US 2011/0117125; I, II, or III of US 2011/0256175; I, II, III, IV, V, VI, VII, VIII, IX, X, XI, XII of US 2012/0202871; I, II, III, IV, V, VI, VII, VIII, X, XII, XIII, XIV, XV, or XVI of US 2011/0076335; I or II of US 2006/008378; I of WO2015/074085 (e.g., ATX-002); I of US 2013/0123338; I or X-A-Y-Z of US 2015/0064242; XVI, XVII, or XVIII of US 2013/0022649; I, II, or III of US 2013/0116307; I, II, or III of US 2013/0116307; I or II of US 2010/0062967; I-X of US 2013/0189351; I of US 2014/0039032; V of US 2018/0028664; I of US 2016/0317458; I of US 2013/0195920; 5, 6, or 10 of U.S. Pat. No. 10,221,127; III-3 of WO 2018/081480; I-5 or I-8 of WO 2020/081938; I of WO 2015/199952 (e.g., compound 6 or 22) and Table 1 therein; 18 or 25 of U.S. Pat. No. 9,867,888; A of US 2019/0136231; II of WO 2020/219876; 1 of US 2012/0027803; OF-02 of US 2019/0240349; 23 of U.S. Pat. No. 10,086,013; cKK-E12/A6 of Miao et al (2020); C₁₂-200 of WO 2010/053572; 7C₁ of Dahlman et al (2017); 304-013 or 503-013 of Whitehead et al; TS-P4C₂ of U 59,708,628; I of WO 2020/106946; I of WO 2020/106946; (1), (2), (3), or (4) of WO 2021/113777; and any one of Tables 1-16 of WO 2021/113777, all of which are incorporated herein by reference in their entirety.

In some embodiments, the lipid-based carrier (or lipid nanoformulation) further includes biodegradable ionizable lipids, for instance, (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). See, e.g., lipids of WO 2019/067992, WO 2017/173054, WO 2015/095340, and WO 2014/136086, which are incorporated herein by reference in their entirety.

### Non-Cationic Lipids (e.g., Phospholipids)

In some embodiments, the lipid-based carrier (or lipid nanoformulation) further comprises one or more non-cationic lipids. In some embodiments, the non-cationic lipid is a phospholipid. In some embodiments, the non-cationic lipid is a phospholipid substitute or replacement. In some embodiments, the non-cationic lipid is a negatively charged (anionic) lipid.

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphocholine (DLPC), Sodium 1,2-ditetradecanoyl-sn-glycero-3-phosphate (DMPA), phosphatidylcholine (lecithin), phosphatidylethanolamine, lysolecithin, lysophosphatidylethanolamine, phosphatidylserine, phosphatidylinositol, sphingomyelin, egg sphingomyelin (ESM), phosphatidylethanolamine (cephalin), cardiolipin, phosphatidic acid, cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C₁₀-C₂₄ carbon chains, e.g., lauroyl, myristoyl, palmitoyl, stearoyl, or oleoyl. Additional exemplary lipids, in certain embodiments, include, without limitation, those described in Kim et al. (2020) dx.doi.org/10.1021/acs.nanolett.0c01386, which is incorporated herein by reference. Such lipids include, in some embodiments, plant lipids found to improve liver transfection with mRNA (e.g., DGTS).

In some embodiments, the lipid-based carrier (or lipid nanoformulation) may comprise a combination of distearoylphosphatidylcholine/cholesterol, dipalmitoylphosphatidylcholine/cholesterol, dimyrystoylphosphatidylcholine/cholesterol, 1,2-Dioleoyl-sn-glycero-3-phosphocholine (DOPC)/cholesterol, or egg sphingomyelin/cholesterol.

Other examples of suitable non-cationic lipids include, without limitation, nonphosphorous lipids such as, e.g., stearylamine, dodecylamine, hexadecylamine, acetyl palmitate, glycerol ricinoleate, hexadecyl stearate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO 2017/099823 or US 2018/0028664, which are incorporated herein by reference in their entirety.

In some embodiments, the lipid-based carrier (or lipid nanoformulation) further comprises one or more non-cationic lipid that is oleic acid or a compound of Formula I, II, or IV of US 2018/0028664, which is incorporated herein by reference in its entirety.

The non-cationic lipid content can be, for example, 0-30% (mol) of the total lipid components present. In some embodiments, the non-cationic lipid content is 5-20% (mol) or 10-15% (mol) of the total lipid components present.

In some embodiments, the lipid-based carrier (or lipid nanoformulation) further comprises a neutral lipid, and the molar ratio of an ionizable lipid to a neutral lipid ranges from about 2:1 to about 8:1 (e.g., about 2:1, 3:1, 4:1, 5:1, 6:1, 7:1, or 8:1).

In some embodiments, the lipid-based carrier (or lipid nanoformulation) does not include any phospholipids. For example, the nanoparticular composition may be substantially free of DSPC.

In some embodiments, the lipid-based carrier (or lipid nanoformulation) can further include one or more phospholipids, and optionally one or more additional molecules of similar molecular shape and dimensions having both a hydrophobic moiety and a hydrophilic moiety (e.g., cholesterol).

Exemplary anionic lipids include dimyrystoyl-, dipalmitoyl-, and distearoyl-phasphatidylglycerol; dimyrystoyl-, dipalmitoyl-, and dipalmitoyl-phosphatidic acid; dimyrystoyl-, dipalmitoyl-, and dipalmitoyl-phosphatidylethanolamine; and their unsaturated diacyl and mixed acyl chain counterparts as well as cardiolipin.

Exemplary neutral lipids include DLPC (1,2-dilauroyl-sn-glycero-3-phosphocholine), DMPC (1,2-dimyristoyl-sn-glycero-3-phosphocholine), DPPC (1,2-dipalmitoyl-sn-glycero-3-phosphocholine), DSPC (1,2-distearoyl-sn-glycero-3-phosphocholine), DOPC (1,2-dioleoyl-sn-glycero-3-phosphocholine), DMPA (Sodium 1,2-ditetradecanoyl-sn-glycero-3-phosphate), DPPE (1,2-Dipalmitoyl-sn-glycero-3-phosphoethanolamine), and DOPE (1,2-dioleoyl-sn-glycero-3-phosphoethanolamine).

Exemplary phospholipids include, but are not limited to, phosphatidylcholine (lecithin), lysolecithin, lysophosphatidylethanol-amine, phosphatidylserine, phosphatidylinositol, sphingomyelin, phosphatidylethanolamine (cephalin), cardiolipin, phosphatidic acid, cerebrosides, dicetylphosphate, phosphatidylcholine, and dipalmitoyl-phosphatidylglycerol.

In some embodiments, the lipid-based carrier (or lipid nanoformulation) further comprises a phospholipid in an amount of 0-25 mol % (e.g., 0-10 mol %, 0-5 mol %, 1-5 mol %, 1-4 mol %, 1-3 mol %, 2-4 mol %, or 2-3 mol %) of the total lipid components.

### Structural Lipids

The lipid-based carrier (or lipid nanoformulation) described herein may further comprise one or more structural lipids. As used herein, the term “structural lipid” refers to sterols (e.g., cholesterol and derivatives thereof) and to lipids containing sterol moieties.

Incorporation of structural lipids in the lipid nanoparticle may help mitigate aggregation of other lipid in the particle. Structural lipids can be selected from the group including but not limited to, cholesterol or cholesterol derivative, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, structural lipids may be incorporated into the lipid-based carrier at molar ratios ranging from about 0.1 to 1.0 (cholesterol phospholipid).

In some embodiments, sterols, when present, can include one or more of cholesterol or cholesterol derivatives, such as those described in WO 2009/127060 or US 2010/0130588, which are incorporated herein by reference in their entirety. Additional exemplary sterols include phytosterols, including those described in Eygeris et al. (2020), Nano Lett. 2020; 20(6):4543-4549, incorporated herein by reference.

In some embodiments, the structural lipid is a cholesterol derivative. Non-limiting examples of cholesterol derivatives include polar analogues such as 5a-cholestanol, 53-coprostanol, cholesteryl-(2′-hydroxy)-ethyl ether, cholesteryl-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryl-(4′-hydroxy)-butyl ether. Exemplary cholesterol derivatives are described in WO 2009/127060 and US 2010/0130588, each of which is incorporated herein by reference in its entirety.

In some embodiments, the lipid-based carrier (or lipid nanoformulation) further comprises sterol in an amount of 0-50 mol % (e.g., 0-10 mol %, 10-20 mol %, 20-50 mol %, 20-30 mol %, 30-40 mol %, or 40-50 mol %) of the total lipid components.

Polymers and Polyethylene Glycol (PEG) Lipids In some embodiments, the lipid-based carrier (or lipid nanoformulation) may include one or more polymers or co-polymers, e.g., poly(lactic-co-glycolic acid) (PFAG) nanoparticles.

In some embodiments, the lipid-based carrier (or lipid nanoformulation) may include one or more polyethylene glycol (PEG) lipid (also referred to as a “PEGylated lipid”). Examples of useful PEG-lipids include, but are not limited to, 1,2-Diacyl-sn-Glycero-3-Phosphoethanolamine-N-[Methoxy(Polyethylene glycol)-350](mPEG 350 PE); 1,2-Diacyl-sn-Glycero-3-Phosphoethanolamine-N-[Methoxy(Polyethylene glycol)-550](mPEG 550 PE); 1,2-Diacyl-sn-Glycero-3-Phosphoethanolamine-N-[Methoxy(Polyethylene glycol)-750](mPEG 750 PE); 1,2-Diacyl-sn-Glycero-3-Phosphoethanolamine-N-[Methoxy(Polyethylene glycol)-1000](mPEG 1000 PE); 1,2-Diacyl-sn-Glycero-3-Phosphoethanolamine-N-[Methoxy(Polyethylene glycol)-2000](mPEG 2000 PE); 1,2-Diacyl-sn-Glycero-3-Phosphoethanolamine-N-[Methoxy(Polyethylene glycol)-3000](mPEG 3000 PE); 1,2-Diacyl-sn-Glycero-3-Phosphoethanolamine-N-[Methoxy(Polyethylene glycol)-5000](mPEG 5000 PE); N-Acyl-Sphingosine-1-[Succinyl(Methoxy Polyethylene Glycol) 750](mPEG 750 Ceramide); N-Acyl-Sphingosine-1-[Succinyl(Methoxy Polyethylene Glycol) 2000](mPEG 2000 Ceramide); and N-Acyl-Sphingosine-1-[Succinyl(Methoxy Polyethylene Glycol) 5000](mPEG 5000 Ceramide). In some embodiments, the PEG lipid is a polyethyleneglycol-diacylglycerol (i.e., polyethyleneglycol diacylglycerol (PEG-DAG), PEG-cholesterol, or PEG-DMB) conjugate.

In some embodiments, the lipid-based carrier (or nanoformulation) includes one or more conjugated lipids (such as PEG-conjugated lipids or lipids conjugated to polymers described in Table 5 of WO 2019/217941, which is incorporated herein by reference in its entirety). In some embodiments, the one or more conjugated lipids is formulated with one or more ionic lipids (e.g., non-cationic lipid such as a neutral or anionic, or zwitterionic lipid); and one or more sterols (e.g., cholesterol).

The PEG conjugate can comprise a PEG-dilaurylglycerol (C12), a PEG-dimyristylglycerol (C14), a PEG-dipalmitoylglycerol (C16), a PEG-disterylglycerol (C18), PEG-dilaurylglycamide (C12), PEG-dimyristylglycamide (C14), PEG-dipalmitoylglycamide (C16), and PEG-disterylglycamide (C18).

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, and those described in Table 2 of WO 2019/051289 (which is herein incorporated by reference in its entirety), and combinations of the foregoing.

Additional exemplary PEG-lipid conjugates are described, for example, in U.S. Pat. Nos. 5,885,613, 6,287,591, US 2003/0077829, US 2003/0077829, US 2005/0175682, US 2008/0020058, US 2011/0117125, US 2010/0130588, US 2016/0376224, US 2017/0119904, US 2018/0028664, and WO 2017/099823, all of which are incorporated herein by reference in their entirety.

In some embodiments, the PEG-lipid is a compound of Formula III, III-a-I, III-a-2, III-b-1, III-b-2, or V of US 2018/0028664, which is incorporated herein by reference in its entirety. In some embodiments, the PEG-lipid is of Formula II of US 2015/0376115 or US 2016/0376224, both of which are incorporated herein by reference in their entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. In some embodiments, the PEG-lipid includes one of the following:

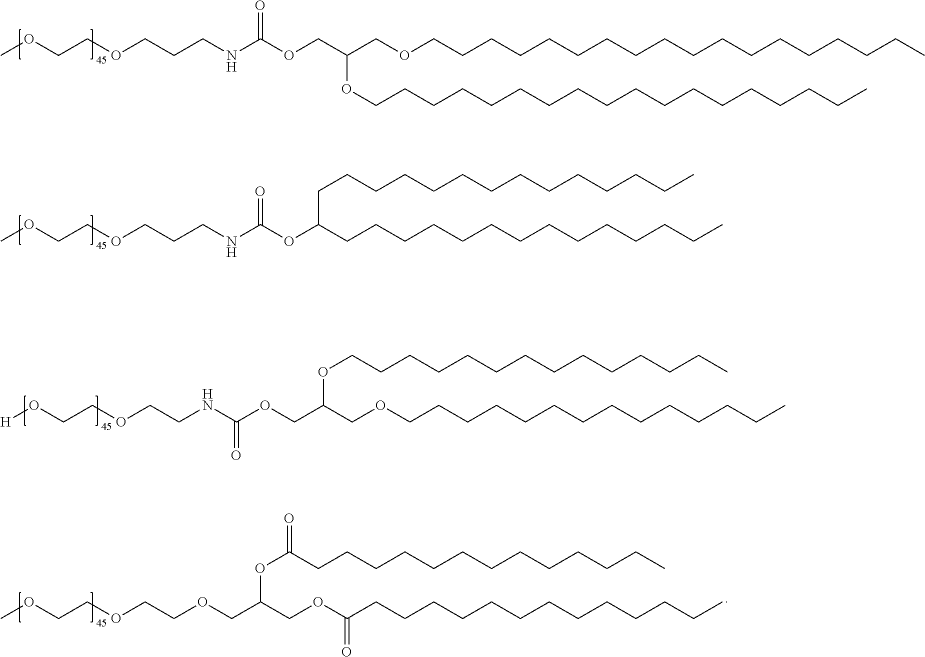

In some embodiments, lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, e.g., PEG-lipids, (POZ)-lipid conjugates, ATTA-lipid conjugates and cationic polymer-lipids, include those described in Table 2 of WO 2019/051289A9, which is incorporated herein by reference in its entirety.

In some embodiments, the conjugated lipid (e.g., the PEGylated lipid) can be present in an amount of 0-20 mol % of the total lipid components present in the lipid-based carrier (or lipid nanoformulation). In some embodiments, the conjugated lipid (e.g., the PEGylated lipid) content is 0.5-10 mol %, 0.5-5 mol %, 0.5-3 mol %, 1-5 mol %, 1-3 mol %, 2-5 mol %, or 2-3 mol %, of the total lipid components.

When needed, the lipid-based carrier (or lipid nanoformulation) described herein may be coated with a polymer layer to enhance stability in vivo (e.g., sterically stabilized LNPs).

Examples of suitable polymers include, but are not limited to, poly(ethylene glycol), which may form a hydrophilic surface layer that improves the circulation half-life of liposomes and enhances the amount of lipid nanoformulations (e.g., liposomes or LNPs) that reach therapeutic targets. See, e.g., Working et al. J Pharmacol Exp Ther, 289: 1128-1133 (1999); Gabizon et al., J Controlled Release 53: 275-279 (1998); Adlakha Hutcheon et al., Nat Biotechnol 17: 775-779 (1999); and Koning et al., Biochim Biophys Acta 1420: 153-167 (1999), which are incorporated herein by reference in their entirety.

In certain embodiments, the nanoparticle composition further comprises a PEGylated lipid, a sterol, a phospholipid, and/or a neutral lipid.

In certain embodiments, the nanoparticle composition further comprises a PEGylated lipid, a sterol, and a phospholipid.

### Percentages of Lipid Nanoformulation Components

In some embodiments, the lipid-based carrier (or lipid nanoformulation) comprises one or more of the lipidoid compounds described herein, optionally a non-cationic lipid (e.g., a phospholipid), a sterol, a neutral lipid, and/or optionally conjugated lipid (e.g., a PEGylated lipid) that inhibits aggregation of particles. The relative amounts of these components can be varied independently and to achieve desired properties. For example, in some embodiments, the ionizable lipid including the lipid compounds described herein is present in an amount from about 20 mol % to about 100 mol % (e.g., 20-90 mol %, 20-80 mol %, 20-70 mol %, 25-100 mol %, 30-70 mol %, 30-60 mol %, 30-40 mol %, 40-50 mol %, or 50-90 mol %) of the total lipid and lipidoid components; a non-cationic lipid (e.g., phospholipid) is present in an amount from about 0 mol % to about 50 mol % (e.g., 0-40 mol %, 0-30 mol %, 5-50 mol %, 5-40 mol %, 5-30 mol %, or 5-10 mol %) of the total lipid and lipidoid components, a conjugated lipid (e.g., a PEGylated lipid) in an amount from about 0.5 mol % to about 20 mol % (e.g., 1-10 mol % or 5-10%) of the total lipid and lipidoid components, and a sterol in an amount from about 0 mol % to about 60 mol % (e.g., 0-50 mol %, 10-60 mol %, 10-50 mol %, 15-60 mol %, 15-50 mol %, 20-50 mol %, 20-40 mol %) of the total lipid and lipidoid components, provided that the total mol % of the lipid component does not exceed 100%.

In some embodiments, the lipid-based carrier (or lipid nanoformulation) comprises about 25-100 mol % of the ionizable lipid including the lipid and lipidoid compounds described herein, about 0-50 mol % phospholipid, about 0-50 mol % sterol, and about 0-10 mol % PEGylated lipid.

In some embodiments, the lipid-based carrier (or lipid nanoformulation) comprises about 25-100 mol % of the ionizable lipid including the lipid and lipidoid compounds described herein; about 0-40 mol % phospholipid (e.g., DSPC), about 0-50 mol % sterol (e.g., cholesterol), and about 0-10 mol % PEGylated lipid.

In some embodiments, the lipid-based carrier (or lipid nanoformulation) comprises about 30-60 mol % (e.g., about 35-55 mol %, or about 40-50 mol %) of the ionizable lipid including the lipid and lipidoid compounds described herein, about 0-30 mol % (e.g., 5-25 mol %, or 10-20 mol %) phospholipid, about 15-50 mol % (e.g., 18.5-48.5 mol %, or 30-40 mol %) sterol, and about 0-10 mol % (e.g., 1-5 mol %, or 1.5-2.5 mol %) PEGylated lipid.

In some embodiments, molar ratios of ionizable lipid/sterol/phospholipid (or another structural lipid)/PEG-lipid/additional components is varied in the following ranges: ionizable lipid (lipidoid) (25-100%); phospholipid (DSPC) (0-40%); sterol (0-50%); and PEG lipid (0-5%).

In some embodiments, the lipid-based carrier (or lipid nanoformulation) comprises the lipidoid in an amount from about 40 mol % to about 55 mol %, the phospholipid in an amount from about 1.0 mol % to about 3 mol %, the sterol in an amount from about 35 mol % to about 50 mol %, and the PEGylated lipid in an amount from about 0.5 mol % to about 3 mol %.

In some aspects, the lipid nanoparticle comprises the lipidoid in an amount from about 45 mol % to about 55 mol %, the phospholipid in an amount from about 1.0 mol % to about 3.0 mol %, the sterol in an amount from about 40 mol % to about 50 mol %; and the PEGylated lipid in an amount from about 1.0 mol % to about 2.0 mol %.

In some aspects, the lipid nanoparticle comprises the lipidoid in an amount of about 51.5 mol %, the phospholipid in an amount of about 2.7 mol %, the sterol in an amount of about 44.5 mol %; and the PEGylated lipid in an amount of about 1.3 mol %.

In exemplary embodiments, the lipid nanoparticle comprises Compound 100 as lipidoid, DSPC as phospholipid, cholesterol as the sterol, and DMG-PEG2k as the PEGylated lipid.

In exemplary embodiments, the lipid nanoparticle comprises the lipidoid Compound 100 in an amount of about 51.5 mol %, the phospholipid is DSPC in an amount of about 2.7 mol %, the sterol is cholesterol in an amount of about 44.5 mol %; and the PEGylated lipid is DMG-PEG2k in an amount of about 1.3 mol %.

In some embodiments, the lipid-based carrier (or lipid nanoformulation) comprises, by mol % or wt % of the total lipid and lipidoid components, 50-75% ionizable lipid (including the lipid and lipidoid compounds as described herein), 20-40% sterol (e.g., cholesterol or derivative), 0 to 10% non-cationic-lipid, and 1-10% conjugated lipid (e.g., the PEGylated lipid).

In some embodiments, the lipidoid compound described herein is a component of the lipid-based carrier (or lipid nanoformulation, or nanoparticle composition) and comprises from 10 mol % to 95 mol %, from 10 mol % to 90 mol %, from 10 mol % to 80 mol %, from 10 mol % to 70 mol %, from 10 mol % to 60 mol %, from 20 mol % to 55 mol %, from 30 mol % to 55 mol %, 35 mol % to 55 mol %, from 40 mol % to 55 mol %, from 45 mol % to 55 mol %, from 48 mol % to 54 mol %, from 49 mol % to 54 mol %, from 50 mol % to 55 mol %, from 50 mol % to 54 mol %, or from 50 mol % to 53 mol % (or any fraction of these ranges) of the total lipid and lipidoid components.

In some embodiments, where the lipid-based carrier (or lipid nanoformulation) contains a mixture of phospholipid and sterol (e.g. cholesterol or derivative), the mixture may be present up to 40 mol %, 45 mol %, 50 mol %, 55 mol %, or 60 mol % of the total lipid and lipidoid components.

In some embodiments, the phospholipid component in the mixture may be present from 2 mol % to 20 mol %, from 2 mol % to 15 mol %, from 2 mol % to 12 mol %, from 2 mol % to 10 mol %, from 2 mol % to 8 mol %, from 2 mol % to 6 mol %, (or any fraction of these ranges) of the total lipid and lipidoid components. In some embodiments, the lipid-based carrier (or lipid nanoformulation or nanoparticle composition) is substantially free of a phospholipid. In certain embodiments, the lipid-based carrier (or lipid nanoformulation or nanoparticle composition) is substantially free of distearolyphosphatidycholine (DSPC).

In some embodiments, the sterol component (e.g. cholesterol or derivative) in the mixture may comprise from 25 mol % to 45 mol %, from 30 mol % to 45 mol %, from 35 mol % to 45 mol %, from 35 mol % to 50 mol %, from 35 mol % to 55 mol %, from 40 mol % to 45 mol %, from 40 mol % to 50 mol %, from 40 mol % to 55 mol %, from 45 mol % to 55 mol %, or from 42 mol % to 47 mol % (or any fraction of these ranges) of the total lipid and lipidoid components.

In some embodiments, where the lipid-based carrier (or lipid nanoformulation) is phospholipid-free, the sterol component (e.g. cholesterol or derivative) may be present up to 25 mol %, 30 mol %, 35 mol %, 40 mol %, 45 mol %, 50 mol %, 55 mol %, or 60 mol % of the total lipid and lipidoid components. For instance, the sterol component (e.g. cholesterol or derivative) may be present from 25 mol % to 65 mol %, from 25 mol % to 60 mol %, from 25 mol % to 55 mol %, from 25 mol % to 50 mol %, from 25 mol % to 45 mol %, from 25 mol % to 40 mol %, from 30 mol % to 45 mol %, from 30 mol % to 40 mol %, from 35 mol % to 45 mol %, from 30 mol % to 35 mol %, or from 35 mol % to 40 mol % (or any fraction thereof or range therein) of the total lipid and lipidoid components.

In some embodiments, the non-ionizable lipid components in the lipid-based carrier (or lipid nanoformulation) may be present from 5 mol % to 90 mol %, from 10 mol % to 85 mol %, or from 20 mol % to 80 mol % (or any fraction of these ranges) of the total lipid and lipidoid components.

The ratio of total lipid components to the cargo (e.g., an encapsulated therapeutic agent such as a nucleic acid) can be varied as desired. For example, the total lipid components to the cargo (mass or weight) ratio can be from about 10:1 to about 30:1. In some embodiments, the total lipid and lipidoid components to the cargo ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, about 6:1 to about 9:1, about 7:1 to about 12:1, or about 10:1. The amounts of total lipid components and the cargo can be adjusted to provide a desired N/P ratio, for example, N/P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the lipid-based carrier (or lipid nanoformulation)'s overall lipid content can range from about 5 mg/mL to about 30 mg/mL. Nitrogen:phosphate ratios (N:P ratio) is evaluated at values between 0.1 and 100.

In some embodiments, the lipid-based carrier (or lipid nanoformulation) includes the ionizable lipid compound (lipidoid) as described herein, phospholipid, cholesterol, and a PEGylated lipid in a molar ratio of 50:10:38.5:1.5. In some embodiments, the lipid-based carrier (or lipid nanoformulation) includes the ionizable lipid compound (lipidoid) as described herein, cholesterol and a PEGylated lipid in a molar ratio of 60:38.5:1.5. In further embodiments, the lipid-based carrier (or lipid nanoformulation) includes the ionizable lipid compound (lipidoid) as described herein, a phospholipid, cholesterol, and a PEGylated lipid in a molar ratio of 51.5:2.7:44.5:1.3.

In some embodiments of any of the aspects or embodiments herein, the lipid-based carrier (or lipid nanoformulation) further comprises a tissue targeting moiety. The tissue targeting moiety can be a peptide, oligosaccharide or the like, which can be used for the delivery of the lipid-based carrier (or lipid nanoformulation) to one or more specific tissues such as the liver. In some embodiments, the tissue targeting moiety is a ligand for liver specific receptors. In some embodiments, the ligand of liver specific receptors used for liver targeting is an oligosaccharide such as N-Acetylgalactosamine (GalNAc) which is covalently attached to a component of a lipid-based carrier (or lipid nanoformulation), e.g., PEG-lipid conjugates or the like. In some embodiments, the GalNAc is covalently attached to, for example, PEG-lipid conjugate. In some embodiments, the GalNAc is conjugated to DSPE-PEG2000. In some embodiments, the GalNAc-PEG-lipid conjugate is present in the lipid-based carrier (or lipid nanoformulation) at a molar percentage of 1.5%, 1.4%, 1.3%, 1.2%, 1.1%, 1.0%, 0.9%, 0.8%, 0.7%, 0.6%, 0.5%, 0.4%, 0.3%, 0.2%, or 0.1% of the total lipid. In some embodiments, the GalNAc-PEG-lipid conjugate is present in the lipid-based carrier (or lipid nanoformulation) at a molar percentage of 0.1% of the total lipid. In some embodiments, the GalNAc-PEG-lipid conjugate is present in the lipid-based carrier (or lipid nanoformulation) at a molar percentage of 0.2% of the total lipid. In some embodiments, the GalNAc-PEG-lipid conjugate is present in the lipid-based carrier (or lipid nanoformulation) at a molar percentage of 0.3% of the total lipid. In some embodiments, the GalNAc-PEG-lipid conjugate is present in the lipid-based carrier (or lipid nanoformulation) at a molar percentage of 0.4% of the total lipid. In some embodiments, the GalNAc-PEG-lipid conjugate is present in the lipid-based carrier (or lipid nanoformulation) at a molar percentage of 0.5% of the total lipid. In some embodiments, the GalNAc-PEG-lipid conjugate is present in the lipid-based carrier (or lipid nanoformulation) at a molar percentage of 0.6% of the total lipid. In some embodiments, the GalNAc-PEG-lipid conjugate is present in the lipid-based carrier (or lipid nanoformulation) at a molar percentage of 0.7% of the total lipid. In some embodiments, the GalNAc-PEG-lipid conjugate is present in the lipid-based carrier (or lipid nanoformulation) at a molar percentage of 0.8% of the total lipid. In some embodiments, the GalNAc-PEG-lipid conjugate is present in the lipid-based carrier (or lipid nanoformulation) at a molar percentage of 0.9% of the total lipid. In some embodiments, the GalNAc-PEG-lipid conjugate is present in the lipid-based carrier (or lipid nanoformulation) at a molar percentage of 1.0% of the total lipid. In some embodiments, the GalNAc-PEG-lipid conjugate is present in the lipid-based carrier (or lipid nanoformulation) at a molar percentage of about 1.5% of the total lipid. In some embodiments, the GalNAc-PEG-lipid conjugate is present in the lipid-based carrier (or lipid nanoformulation) at a molar percentage of 2.0% of the total lipid.

### Properties of Lipid Nanoformulations

In some embodiments, the average particle diameter of the lipid-based carrier (or lipid nanoformulation) may be between 10s of nm and 100s of nm, e.g., measured by dynamic light scattering (DLS). In some embodiments, the average particle diameter of the lipid-based carrier (or lipid nanoformulation) ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, from about 38 mm to about 42 mm, from about 40 nm to about 150 nm (such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm), from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm.

The lipid-based carrier or lipid nanoformulation (e.g., liposome or LNP) may, in some instances, be relatively homogenous. A polydispersity index may be used to indicate the homogeneity of a lipid nanoformulation (e.g., liposome or LNP), e.g., the particle size distribution of the liposome or LNP. A small (e.g., less than 0.3) polydispersity index generally indicates a narrow particle size distribution. A lipid-based carrier or lipid nanoformulation (e.g., liposome or LNP) may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of the lipid-based carrier or lipid nanoformulation (e.g., liposome or LNP) may be from about 0.10 to about 0.20.

The zeta potential of a lipid-based carrier or a lipid nanoformulation (e.g., liposome or LNP) may be used to indicate the electrokinetic potential of the composition. In some embodiments, the zeta potential may describe the surface charge of a liposome or LNP. Lipid nanoformulations (e.g., liposomes or LNP) with relatively low charges, positive or negative, are generally desirable, as more highly charged species may interact undesirably with cells, tissues, and other elements in the body. In some embodiments, the zeta potential of a liposome or LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a cargo such as a protein and/or nucleic acid, describes the amount of protein and/or nucleic acid that is encapsulated or otherwise associated with a lipid nanoformulation (e.g., liposome or LNP) after preparation, relative to the initial amount provided. The encapsulation efficiency is desirably high (e.g., at least 70%, 80%, 90%, 95%, or close to 100%). The encapsulation efficiency may be measured, for example, by comparing the amount of protein or nucleic acid in a solution containing the liposome or LNP before and after breaking up the liposome or LNP with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the liposome or LNP described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

The lipid carrier or lipid nanoformulation may optionally include one or more coatings. In some embodiments, the lipid carrier or lipid nanoformulation (e.g., liposome or LNP) may be formulated in a capsule, film, or tablet having a coating. A capsule, film, or tablet including a composition described herein may have any useful size, tensile strength, hardness, or density.

Additional exemplary lipids, formulations, methods, and characterization of a lipid carrier or lipid nanoformulation (e.g., liposome or LNP) are taught by WO 2020/061457 and WO 2021/113777, which are incorporated herein by reference in their entirety. Further exemplary lipids, formulations, methods, and characterization of LNPs are taught by Hou et al. Lipid nanoparticles for mRNA delivery. Nat Rev Mater (2021). doi.org/10.1038/s41578-021-00358-0, which is incorporated herein by reference in its entirety (see, for example, exemplary lipids and lipid derivatives of FIG. 2 of Hou et al.).

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

Lipid nanoformulations (e.g., liposome or LNP) optimized for the delivery of CRISPR-Cas systems, e.g., Cas9-gRNA RNP, gRNA, Cas9 mRNA, are described in WO 2019067992 and WO 2019067910, which are incorporated by reference in their entirety.

Additional specific lipid nanoformulations (e.g., liposome or LNP) useful for delivery of nucleic acid effector molecules are described in U.S. Pat. Nos. 8,158,601 and 8,168,775, which are incorporated by reference in their entirety.

A variety of methods can be used for preparing the lipid carrier or lipid nanoformulation (e.g., liposomes or LNPs) described herein. Such methods are known in the art or disclosed herein, for example, the methods described in Lichtenberg and Barenholz in Methods of Biochemical Analysis, 33:337-462 (1988), which is incorporated herein by reference in its entirety. See also Szoka et al., Ann. Rev. Biophys. Bioeng. 9:467 (1980); U.S. Pat. Nos. 4,235,871; 4,501,728; and 4,837,028; Liposomes, Marc J. Ostro, ed., Marcel Dekker, Inc., New York, 1983, Chapter 1; and Hope, et al., Chem. Phys. Lip. 40:89 (1986), which are incorporated herein by reference in their entirety. Small unilamellar vesicles (SUV, size<100 nm) can be prepared by a combination of standard methods of thin-film hydration and repeated extrusion.

Techniques for sizing the lipid carrier or lipid nanoformulations (e.g., liposomes or LNPs) to a desired size are well-known to one skilled in the art. See, e.g., U.S. Pat. No. 4,737,323, and Hope et al., Biochim. Biophys. Acta, 812: 55-65, which are incorporated by reference in their entirety. Sonicating a lipid nanoformulation (e.g., liposome or LNP) suspension either by bath or probe sonication produces a progressive size reduction down to small unilamellar vesicles less than about 50 nm in size. Homogenization or microfluidization are other methods which rely on shearing energy to fragment large lipid nanoformulations (e.g., liposomes or LNPs) into smaller ones. In a typical homogenization procedure, multilamellar vesicles are recirculated through a standard emulsion homogenizer until selected lipid nanoformulation (e.g., liposome or LNP) sizes, typically between about 100 and 500 nm, are observed. In both methods, the particle size distribution can be monitored by conventional laser-beam particle size discrimination.

Extrusion of lipid nanoformulations (e.g., liposomes or LNPs) through a small-pore polycarbonate membrane or an asymmetric ceramic membrane is a very effective method for reducing liposome or LNP sizes to a relatively well-defined size distribution. Typically, the suspension is cycled through the membrane one or more times until the desired liposome or LNP size distribution is achieved. The lipid-based carrier or lipid nanoformulations may be extruded through successively smaller-pore membranes, to achieve a gradual reduction in liposome or LNP size.

Any of the lipid-based carrier or lipid nanoformulations described herein can be analyzed by methods well-known to one skilled in the art to determine its physical and/or chemical features. For example, a phosphate assay can be used to determine the concentration of the lipid nanoformulations. One phosphate assay is based on the interaction between molybdate and malachite green dye. The main principle involves the reaction of inorganic phosphate with molybdate to form a colorless unreduced phosphomolybdate complex which is converted to a blue colored complex when reduced under acidic conditions. Phosphomolybdate gives 20 or 30 times more color when complexed with malachite green. The final product, reduced green soluble complex is measured by its absorbance at 620 nm and is a direct measure of inorganic phosphate in solution.

In some embodiments, the lipid-based carrier or lipid nanoformulations disclosed herein are tested for particle size, lipid concentration, and active agent encapsulation.

### Therapeutic Agents and Other Cargo

In certain embodiments, the nanoparticle composition further comprises a therapeutic agent.

In certain such embodiments, the therapeutic agent is a nucleic acid molecule, such as a plasmid, an immunostimulatory oligonucleotide, an antisense oligonucleotide, an antagomir, an aptamer, a deoxyribozyme (DNAzyme), or a ribozyme. In certain embodiments, the nucleic acid molecule is DNA or RNA. In certain embodiments, the nucleic acid molecule is DNA; and the DNA is a linear DNA, circular DNA, single stranded DNA, or double stranded DNA. In alternative embodiments, wherein the nucleic acid molecule is RNA; the RNA is messenger RNA (mRNA), miRNA, siRNA or siRNA precursor, RNA aptamer, linear RNA, circular RNA, single stranded RNA, double stranded RNA, tRNA, microRNA (miRNA) or miRNA precursor, Dicer substrate small interfering RNA (dsiRNA), Dicer substrate RNA (dsRNA), short hairpin RNA (shRNA), asymmetric interfering RNA (aiRNA), guide RNA (gRNA), lncRNA, ncRNA, sncRNA, rRNA, snRNA, piRNA, snoRNA, snRNA, scaRNA, exRNA, scaRNA, Y RNA, or hnRNA, preferably mRNA. In yet further embodiments, the nucleic acid molecule comprises a phosphoramide, a phosphorothioate, a phosphorodithioate, an O-methylphosphoroamidate, a morpholino, a locked nucleic acid (LNA), a glycerol nucleic acid (GNA), a threose nucleic acid (TNA), or a peptide nucleic acid (PNA).

In some embodiments, the nucleic acid molecule (e.g., RNA or DNA) encodes a therapeutic peptide or polypeptide, operably linked to a promoter for a DNA. The therapeutic peptide or polypeptide may be, e.g., a transcription factor; a chromatin remodeling factor; an antigen; a hormone; an enzyme (such as a nuclease, e.g., an endonuclease, e.g., a nuclease element of a CRISPR system, e.g., a Cas9, dCas9, aCas9-nickase, Cpf/Cas12a); a Crispr-linked enzyme, e.g., a base editor or prime editor; a mobile genetic element protein (e.g., a transposase, a retrotransposase, a recombinase, an integrase); a Gene Writer; a polymerase; a methylase; a demethylase; an acetylase; a deacetylase; a kinase; a phosphatase; a ligase; a deubiquitinase; an integrase; a recombinase; a topoisomerase; a gyrase; a helicase; a lysosomal acid hydrolase); an antibody; a receptor ligand; a receptor; a clotting factor; a membrane protein; a mitochondrial protein; a nuclear protein; an antibody or other protein scaffold binder such as a centyrin, darpin, or adnectin.

In some embodiments, the nucleic acid molecule (e.g., a DNA or RNA) encodes (if DNA) or is (if RNA) a non-coding RNA, e.g., one or more of a siRNA, a miRNA, long non-coding RNA, a piRNA, a snoRNA, a scaRNA, a tRNA, a rRNA, a therapeutic RNA aptamer, and a snRNA.

In some embodiments, the nucleic acid molecule can include a plurality of sequences. The plurality may be the same or different types. The plurality of sequences may be the same or different sequences of the same type.

All the nucleic acid molecules described herein can be chemically modified. The various modification strategy to the nucleic acid molecules are well known to one skilled in the art. In some embodiments, the nucleic acid molecule comprises one or more modifications such as pseudouridine, 5-bromouracil, 5-methylcytosine, peptide nucleic acid, xeno nucleic acid, morpholinos, locked nucleic acids, glycol nucleic acids, threose nucleic acids, dideoxynucleotides, cordycepin, 7-deaza-GTP, florophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudourdine, dihydrouridine, queuosine, or wyosine. In some embodiments, the antisense oligonucleotide may be a locked nucleic acid oligonucleotide (LNA). The term “locked nucleic acid (LNA)” refers to oligonucleotides that contain one or more nucleotide building blocks in which an extra methylene bridge fixes the ribose moiety either in the C3′-endo (beta-D-LNA) or C2′-endo (alpha-L-LNA) conformation (Grunweller A, Hartmann R K, BioDrugs, 21(4): 235-243 (2007)).

Additional examples of the nucleic acid molecules (including tumor suppressor genes, antisense oligonucleotides, siRNA, miRNA, or shRNA) may be found in U.S. Published Patent Application No. 2007/0065499 and U.S. Pat. No. 7,780,882, which are incorporated by reference herein in their entireties.

In some embodiments, the nanoparticle composition can include a plurality of nucleic acid molecules, which may be the same or different types.

The N:P ratio of an LNP indicates the molar ratio between the amine groups (N) of the ionizable lipid and the phosphate groups (P) of the nucleic acid cargo. The N:P ratio can be expressed as a ratio of two numbers or as a single digit (e.g., 10:1 and 10 are equivalent N:P ratios). In some embodiments, the N:P ratio of the nucleic acid molecule-lipidoid composition ranges from 1:1 to 30:1, for instance from 3:1 to 20:1, from 3:1 to 15:1, from 3:1 to 10:1, or from 3:1 to 6:1. An N:P ratio refers to the molar ratio of the amines present in the plurality of lipidoids to the phosphates present in the nucleic acid molecule. In certain embodiments, the N:P ratio of the nucleic acid molecule-lipidoid composition ranges from 3:1 to 15:1, or about 5:1 to 15:1, or about 7:1 to about 12:1. In certain embodiments, the N:P ratio of the LNPs described herein is about 6 (i.e., 6:1). In other embodiments, the N:P ratio of the LNPs described herein is about 10 (i.e., 10:1).

In alternative embodiments, the therapeutic agent is a protein or small molecule drug.

In certain embodiments, the protein may be a peptide or polypeptide, e.g., a transcription factor; a chromatin remodeling factor; an antigen; a hormone; an enzyme (such as a nuclease, e.g., an endonuclease, e.g., a nuclease element of a CRISPR system, e.g., a Cas9, dCas9, aCas9-nickase, Cpf/Cas12a); a Crispr-linked enzyme, e.g., a base editor or prime editor; a mobile genetic element protein (e.g., a transposase, a retrotransposase, a recombinase, an integrase); a gene writer; a polymerase; a methylase; a demethylase; an acetylase; a deacetylase; a kinase; a phosphatase; a ligase; a deubiquitinase; an integrase; a recombinase; a topoisomerase; a gyrase; a helicase; a lysosomal acid hydrolase); an antibody; a receptor ligand; a receptor; a clotting factor; a membrane protein; a mitochondrial protein; a nuclear protein; an antibody or other protein scaffold binder, such as a centyrin, darpin, or adnectin.

In certain embodiments, the protein is a ribonucleoprotein (RNP) that a complex of ribonucleic acid and RNA-binding protein.

In certain embodiments, the protein is a recombinant cytokine such as IL-12.

In certain embodiments, the nanoparticle composition can include a plurality of protein molecules, which may be the same or different types.

In certain embodiments, the small molecule drug comprises an immune suppressive agent, an endosomal escape agent, or a combination of both.

In certain embodiments, the small molecule drug is an HDAC inhibitor, a kinase inhibitor, a cytotoxic molecule, a chromatin modulator, an RNAi modulator, transcription factor, an adjuvant, or a combination of two or more.

In some embodiments, the small molecule drug may be a compound with limited cell permeability properties. For instance, the small molecule drug may be a STING (simulator of interferon genes) modulator.

In some embodiments, the nanoparticle composition can include a plurality of small molecule drugs, which may be the same or different types.

In some embodiments, the therapeutic agent may be encapsulated in the LNP. For example, the therapeutic agent may be completely or partially located in the interior space of the LNPs, within the lipid layer/membrane, or associated with the exterior surface of the lipid layer/membrane. In some embodiments, incorporation of the therapeutic agent into the LNP protects the therapeutic agents from environments which may contain enzymes or chemicals or conditions that degrade the therapeutic agents and/or systems or receptors that cause the rapid excretion of the therapeutic agents. Moreover, incorporating the therapeutic agent into the LNP may promote uptake of the therapeutic agent, and hence, may enhance the therapeutic effect.

The ratio of total lipid and lipidoid components to the therapeutic agent can be varied as desired. For example, the total lipid and lipidoid components to the therapeutic agent (mass or weight) ratio can be from about 10:1 to about 30:1. In some embodiments, the total lipid and lipidoid components to the therapeutic agent ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of total lipid and lipidoid components and the therapeutic agent can be adjusted to provide a desired N:P ratio, for example, N:P ratio of 3, 4, 5, 6, 7, 8, 9, 10, 11, 12, 13, 14, 15, 16, 17, 18, 19, 20, 21, 22, 23, 24, 25, 26, 27, 28, 29, 30, or higher. Generally, the overall lipid and lipidoid content can range from about 5 mg/mL to about 30 mg/mL in the nanoparticle composition.

The nanoparticle composition may contain about 5 to about 95% by weight of the therapeutic agent. In some embodiments, the nanoparticle composition contains about 5%, about 10%, about 20, about 30, about 40, about 50, about 60, about 70, about 80, about 90, or about 95% by weight of the therapeutic agent. In some embodiments, the nanoparticle composition contains the therapeutic agent in an amount about 5-95%, about 5-90%, about 5-80%, about 5-70%, about 5-60%, about 5-50%, about 5-40%, about 5-30%, about 5-20%, about 5-10%, about 10-95%, about 10-90%, about 10-80%, about 10-70%, about 10-60%, about 10-50%, about 10-40%, about 10-30%, about 10-20%, about 20-95%, about 20-90%, about 20-80%, about 20-70%, about 20-60%, about 20-50%, about 20-40%, about 20-30%, about 30-95%, about 30-90%, about 30-80%, about 30-70%, about 30-60%, about 30-50%, about 30-40%, about 40-95%, about 40-90%, about 40-80%, about 40-70%, about 40-60%, about 40-50%, about 50-95%, about 50-90%, about 50-80%, about 50-70%, about 50-60%, about 60-95%, about 60-90%, about 60-80%, about 60-70%, about 70-95%, about 70-90%, about 70-80%, about 80-95%, about 80-90%, or about 90-95%, based on the weight of the nanoparticle composition.

In certain embodiments, the nanoparticle composition further comprises an antigen; wherein the antigen is a protein or a nucleic acid; the antigen is a protein; or the antigen is a nucleic acid.

In further embodiments, the nanoparticle composition further comprises an mRNA molecule comprising a nucleotide sequence that encodes an antigen.

The nanoparticle compositions described herein are useful for delivering a therapeutic agent. Accordingly, in certain embodiments, provided herein is a method of delivering a therapeutic agent, comprising administering to a subject in need thereof an effective amount of the nanoparticle composition of the invention that comprises a therapeutic agent.

The nanoparticle compositions described herein are useful for delivering an antigen. Accordingly, in certain embodiments, provided herein is a method of vaccination, comprising administering to a subject in need thereof an effective amount of a nanoparticle composition of the invention, wherein the nanoparticle composition comprises an antigen. In further embodiments, provided herein is a method of vaccination, comprising administering to a subject in need thereof an effective amount of a nanoparticle composition of the invention, wherein the nanoparticle composition further comprises an mRNA molecule comprising a nucleotide sequence that encodes an antigen.

### Pharmaceutical Compositions

In certain aspects, the present invention provides a pharmaceutical composition comprising a nanoparticle composition of the invention, in combination with a pharmaceutically acceptable carrier.

As used throughout this section, a “lipidoid composition” can refer to a nanoparticle composition comprising a lipidoid compound, e.g., a lipidoid compound of the invention.

The compositions and methods of the present invention may be utilized to treat an individual in need thereof. The pharmaceutical composition described herein may comprise a therapeutic or prophylactic composition, or any combination thereof. In certain embodiments, the individual is a mammal such as a human, or a non-human mammal. When administered to an animal, such as a human, the nanoparticle composition or the lipidoid composition is preferably administered as a pharmaceutical composition comprising, for example, a lipidoid composition and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a lipidoid composition. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a lipidoid composition. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those lipidoid compositions, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The lipidoid composition may also be formulated for inhalation. In certain embodiments, a lipidoid composition may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the lipidoid composition which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active composition, such as a lipidoid (e.g., nanoparticle) composition as described herein, with the carrier and, optionally, one or more accessory ingredients. In general, the formulations are prepared by uniformly and intimately bringing into association a lipidoid (e.g., nanoparticle) composition as described herein with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a lipidoid (e.g., nanoparticle) composition as described herein of the present invention as an active ingredient. Lipidoid compositions may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium lipidoid compositions; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered lipidoid composition moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active lipidoid compositions, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active lipidoid composition may be mixed under sterile conditions with a pharmaceutically acceptable carrier, and with any preservatives, buffers, or propellants that may be required.

The ointments, pastes, creams and gels may contain, in addition to an active lipidoid composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active lipidoid composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a lipidoid composition to the body. Such dosage forms can be made by dissolving or dispersing the active lipidoid composition in the proper medium. Absorption enhancers can also be used to increase the flux of the lipidoid composition across the skin. The rate of such flux can be controlled by either providing a rate controlling membrane or dispersing the lipidoid composition in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active lipidoid compositions in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the lipidoid compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active lipidoid compositions can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a lipidoid composition at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular lipidoid composition or combination of lipidoid compositions employed, or the ester, salt or amide thereof, the route of administration, the time of administration, the rate of excretion of the particular lipidoid composition(s) being employed, the duration of the treatment, other drugs, lipidoid compositions and/or materials used in combination with the particular lipidoid composition(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or lipidoid composition at levels lower than that required in order to achieve the desired therapeutic effect and gradually increase the dosage until the desired effect is achieved. By “therapeutically effective amount” is meant the concentration of a lipidoid composition that is sufficient to elicit the desired therapeutic effect. It is generally understood that the effective amount of the lipidoid composition will vary according to the weight, sex, age, and medical history of the subject. Other factors which influence the effective amount may include, but are not limited to, the severity of the patient's condition, the disorder being treated, the stability of the lipidoid composition, and, if desired, another type of therapeutic agent being administered with the lipidoid composition of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active lipidoid composition used in the compositions and methods of the invention will be that amount of the lipidoid composition that is the lowest dose effective to produce a therapeutic effect. Such an effective dose will generally depend upon the factors described above.

If desired, the effective daily dose of the active lipidoid composition may be administered as one, two, three, four, five, six or more sub-doses administered separately at appropriate intervals throughout the day, optionally, in unit dosage forms. In certain embodiments of the present invention, the active lipidoid composition may be administered two or three times daily. In preferred embodiments, the active lipidoid composition will be administered once daily.

The patient receiving this treatment is any animal in need, including primates, in particular humans; and other mammals such as equines, cattle, swine, sheep, cats, and dogs; poultry; and pets in general.

In certain embodiments, lipidoid compositions of the invention may be used alone or conjointly administered with another type of therapeutic agent.

The present disclosure includes the use of pharmaceutically acceptable salts of lipidoids of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or a viral infection. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, heteroaryl, cycloalkyl, heterocycloalkyl, amino, alkylamino, dialkylamino, amido (—C(O)NH₂, —C(O)NH(alkyl), —C(O)N(alkyl)₂), carboxyl (—C(O)OH), cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted. —C(O)NH₂,

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted. A “linear” alkyl group refers to a straight-chain alkyl group without a branching point. A “branched” alkyl group refers to an alkyl group having at least one branch point. Examples of branched alkyl groups include, e.g., isopropyl, sec-butyl, and tert-butyl.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “alkylene” refers to a divalent alkyl group. The term (hydroxyalkyl)-O-alkylene-, for example, refers to a divalent alkyl group (i.e., an alkylene), appended to the parent molecule at one of its open valencies, and attached to an oxygen atom at the other of its open valencies, which oxygen atom is substituted by a hydroxyalkyl group.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkenyl” as used herein means a straight or branched chain hydrocarbon radical containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl. The unsaturated bond(s) of the alkenyl group can be located anywhere in the moiety and can have either the (Z) or the (E) configuration about the double bond(s).

The term “alkynyl” as used herein means a straight or branched chain hydrocarbon radical containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—, preferably alkylC(O)NH—.

The term “acyloxy” is art-recognized and refers to a group represented by the general formula hydrocarbylC(O)O—, preferably alkylC(O)O—.

The term “alkoxy” refers to an alkyl group appended to the parent molecular moiety through an oxygen atom. Representative alkoxy groups include methoxy, ethoxy, propoxy, tert-butoxy and the like.

The term “alkoxyalkyl” refers to an alkyl group substituted with an alkoxy group and may be represented by the general formula -alkyl-O-alkyl.

The term “alkylamino”, as used herein, refers to an amino group substituted with at least one alkyl group. A “dialkylamino” refers to an amino group substituted with two alkyl groups.

The term “alkylthio”, as used herein, refers to a thiol group substituted with an alkyl group and may be represented by the general formula alkylS—.

The term “amide” or “amido”, as used herein, refers to a group

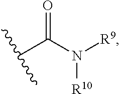

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure. In some embodiments, “amido” refers to the group —C(O)NH₂. In further embodiments, “amido” refers to the group —C(O)N(alkyl)₂ or —C(O)NH(alkyl).

The terms “amine” and “amino” are art-recognized and refer to both unsubstituted and substituted amines and salts thereof, e.g., a moiety that can be represented by

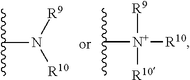

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure. In some embodiments, “amino” refers to the group —NH₂.

The term “aminoalkyl”, as used herein, refers to an alkyl group substituted with an amino group.

The term “arylalkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “arylene” refers to a divalent aryl group.

The term “carbamate” is art-recognized and refers to a group

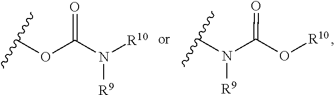

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy” or “carboxyl”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Cycloalkyl groups include, e.g., cyclopropyl, cyclobutyl, cyclopentyl, and cyclohexyl.

The term “cycloalkylene” refers to a divalent cycloalkyl group.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “heteroaralkyl” and “heteroarylalkyl”, as used herein, refers to an alkyl group substituted with a heteroaryl group.

The term “heteroaryl” includes substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroarylene” refers to a divalent heteroaryl group.

The term “heteroatom” as used herein means an atom of any element other than carbon or hydrogen. Preferred heteroatoms are nitrogen, oxygen, and sulfur.

The terms “heterocycloalkylalkyl” and “heterocyclylalkyl”, as used herein, refers to an alkyl group substituted with a heterocycle group.

The terms “heterocycloalkyl,” “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocycloalkyl,” “heterocyclyl”, “heterocycle”, and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocycloalkyl groups include, for example, piperidine, piperazine, pyrrolidine, morpholine, lactones, lactams, and the like. In certain embodiments, heterocyclyl can be monovalent. In other embodiments, heterocyclyl can be bivalent.

The term “heterocyclylene” refers to a divalent heterocyclyl group.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

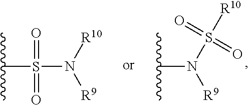

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹
wherein R⁹ represents a hydrocarbyl.

The term “thioether”, as used herein, is equivalent to an ether, wherein the oxygen is replaced with a sulfur.

The term “urea” is art-recognized and may be represented by the general formula

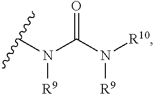

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds disclosed herein. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds disclosed herein. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

In certain embodiments, the lipidoid compositions (e.g., nanoparticles) useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Some of the lipidoid compositions (e.g., nanoparticles) may also comprise chemical compound which exist in tautomeric forms. Such forms, although not explicitly indicated in the formulae described herein, are intended to be included within the scope of the present disclosure.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “ionizable lipid” refers to a molecule having both an ionizable and lipophilic component. “Ionizable” means a group contained in the lipid (e.g., a head group) can be ionized, e.g., dissociated to produce one or more electrically charged species, under a given condition (e.g., pH). For instance, an ionizable lipid may carry a net positive charge at a selected pH, such as physiological pH (e.g., pH of about 7.0). In some embodiments, the hydrophilic component contains an ionizable amine. In some embodiments, the hydrophobic component contains one or more linear or branched lipids.

A “lipid nanoparticle” or “LNP” refers to a composition comprising a lipid or lipidoid (e.g., ionic (e.g., cationic or anionic), zwitterionic, or ionizable lipid) for encapsulation of a cargo. LNPs may also include neutral lipids such as phospholipid molecules belonging to the phosphatidylcholine (PC) class; sterols, such as cholesterol; and polyethylene glycol (PEG). LNPs may be taken up by cells via endocytosis and the ionizability of the lipids at low pH enables endosomal escape, which can allow release of cargo into the cytoplasm. LNPs are liposome-like structures. However, LNPs may not have a contiguous bilayer; some LNPs may have a single phospholipid outer layer encapsulating the interior assuming a micelle-like structure, which can have a non-aqueous core. Exemplary lipid nanoparticle composition are formulations of ionizable lipids, sterols (or hydrophobic molecules), structural lipids such as phospholipids, polyethyleneglycol (PEG) lipids, and potentially additional components (see Nature Nanotechnology 15:313-320 (2020), which is incorporated herein by reference in its entirety), or single molecules containing combinations of ionizable lipid, sterol, structural phospholipid, and shielding groups (see Nature Materials 20:701-710 (2021), which is incorporated herein by reference in its entirety). These components may be mixed with a therapeutic agent (e.g., nucleic acid molecules such as mRNA) to be formulated into LNP composition.

“Nucleic acid” means an oligonucleotide or polynucleotide sequence. Non-limiting examples of oligonucleotide or polynucleotides are DNA, plasmid DNA, self-amplifying RNA, mRNA, siRNA and tRNA. The term also encompasses RNA/DNA hybrids. Nucleotides are typically linked in a nucleic acid by phosphodiester bonds, although the term “nucleic acid” also encompasses nucleic acid analogs having other types of linkages or backbones (e.g., phosphoramide, phosphorothioate, phosphorodithioate, O-methylphosphoroamidate, morpholino, locked nucleic acid (LNA), glycerol nucleic acid (GNA), threose nucleic acid (TNA), and peptide nucleic acid (PNA) linkages or backbones, among others). The nucleic acids may be single-stranded, double-stranded, or contain portions of both single-stranded and double-stranded sequence. A nucleic acid can contain any combination of deoxyribonucleotides and ribonucleotides, as well as any combination of bases, including, for example, adenine, thymine, cytosine, guanine, uracil, and modified or non-canonical bases (including, e.g., hypoxanthine, xanthine, 7-methylguanine, 5,6-dihydrouracil, 5-methylcytosine, and 5 hydroxymethylcytosine).

As used herein, an “RNA” refers to a ribonucleic acid that may be naturally or non-naturally occurring. For example, an RNA may include modified and/or non-naturally occurring components such as one or more nucleobases, nucleosides, nucleotides, or linkers. An RNA may include a cap structure, a chain terminating nucleoside, a stem loop, a polyA sequence, and/or a polyadenylation signal. An RNA may have a nucleotide sequence encoding a polypeptide of interest. For example, an RNA may be a messenger RNA (mRNA). Translation of an mRNA encoding a particular polypeptide, for example, in vivo translation of an mRNA inside a mammalian cell, may produce the encoded polypeptide. RNAs may be selected from the non-limiting group consisting of small interfering RNA (siRNA), asymmetrical interfering RNA (aiRNA), microRNA (miRNA), Dicer-substrate RNA (dsRNA), small hairpin RNA (shRNA), mRNA, and mixtures thereof.

In some embodiments, the therapeutic agent is an antisense nucleic acid. The term “antisense nucleic acid” refers to a non-enzymatic nucleic acid molecule that binds to target RNA by means of RNA-RNA or RNA-DNA or RNA-PNA (protein nucleic acid) interactions and alters the activity of the target RNA. Typically, antisense molecules are complementary to a target sequence along a single contiguous sequence of the antisense molecule. However, in certain embodiments, an antisense molecule can bind to substrate such that the substrate molecule forms a loop, and/or an antisense molecule can bind such that the antisense molecule forms a loop. Thus, the antisense molecule can be complementary to two (or even more) non-contiguous substrate sequences or two (or even more) non-contiguous sequence portions of an antisense molecule can be complementary to a target sequence or both. In addition, antisense DNA can be used to target RNA by means of DNA-RNA interactions, thereby activating RNase H, which digests the target RNA in the duplex.

### EXAMPLES

The invention now being generally described, it will be more readily understood by reference to the following examples which are included merely for purposes of illustration of certain aspects and embodiments of the present invention, and are not intended to limit the invention.

### Example 1. Synthesis and Characterization of Certain Exemplary Compounds

### General Procedure for Synthesis of Compound 100:

### General Procedure for Preparation of Compound 5:

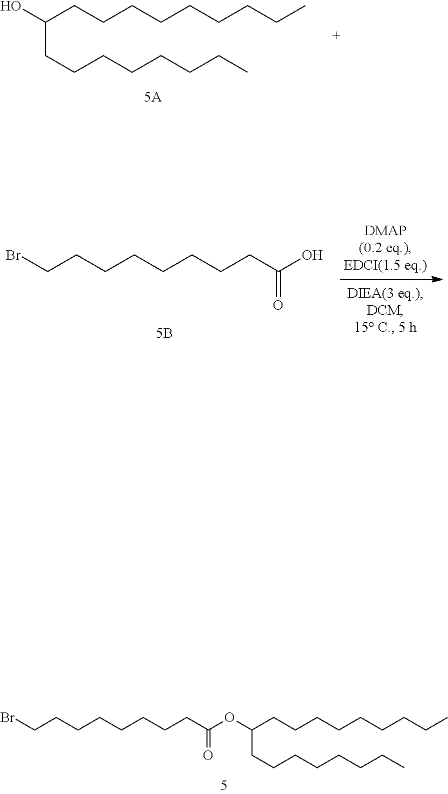 [SMILES: CCCCCCCCCC(O)CCCCCCCC ; O=C(O)CCCCCCCCBr ; CCCCCCCCCC(CCCCCCCC)OC(=O)CCCCCCCCBr]

To a solution of Compound 5A (4.56 g, 16.87 mmol, 0.8 eq.) and Compound 5B (5 g, 21.09 mmol, 1 eq.) in DCM (50 mL) was added DMAP (515.18 mg, 4.22 mmol, 0.2 eq.), EDCI (6.06 g, 31.63 mmol, 1.5 eq.) and DIEA (8.18 g, 63.26 mmol, 11.02 mL, 3 eq.). The mixture was stirred at 15° C. for 5 hours. TLC (Petroleum ether:Ethyl acetate=5:1, R_{f}=0.65) indicated Compound 5A was consumed completely and one new spot formed. The reaction mixture was concentrated under reduced pressure to give a residue. The residue was purified by flash column (SiO₂, Petroleum ether:Ethyl acetate=60:1) to give Compound 5 (6.9 g, 14.09 mmol, 66.84% yield) as a yellow oil.

¹H NMR (400 MHz, CHLOROFORM-d) δ=4.87 (quin, J=6.4 Hz, 1H), 3.45-3.38 (m, 2H), 2.29 (t, J=7.6 Hz, 2H), 1.91-1.81 (m, 2H), 1.67-1.58 (m, 2H), 1.51 (br d, J=5.6 Hz, 4H), 1.46-1.39 (m, 2H), 1.34-1.23 (m, 32H), 0.89 (t, J=6.8 Hz, 6H).

### General Procedure for Preparation of Compound 3:

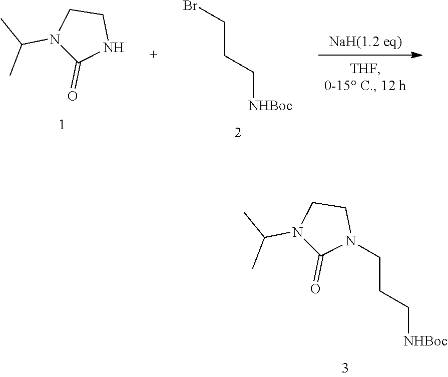

To a solution of Compound 1 (500 mg, 3.90 mmol, 1 eq.) in THF (50 mL) was added NaH (187.23 mg, 4.68 mmol, 60% purity, 1.2 eq.) at 0° C., and the reaction was stirred for 0.5 h. Then, Compound 2 (2.14 g, 8.97 mmol, 2.3 eq.) was added at 0° C. The reaction mixture was stirred at 15° C. for 12 hours. TLC (Ethyl acetate, R_{f}=0.4) showed Compound 1 was consumed and new spots formed. The reaction was added saturated NH₄Cl (20 mL) and extracted with ethyl acetate (20 mL×2). The combined organic layers were concentrated to give a residue. The residue was purified by flash column (SiO₂, Petroleum ether:Ethyl acetate=100:1 to 0:1) to give Compound 3 (500 mg, 1.75 mmol, 44.91% yield) as a yellow oil.

LCMS [M+1]⁺=286.3

¹H NMR (400 MHz, CHLOROFORM-d) δ=5.45-5.06 (m, 1H), 4.13 (td, J=6.8, 13.6 Hz, 1H), 3.35-3.20 (m, 6H), 3.12 (t, J=6.4 Hz, 2H), 1.65 (quin, J=6.4 Hz, 2H), 1.44 (s, 9H), 1.12 (d, J=6.8 Hz, 6H).

### General Procedure for Preparation of Compound 4:

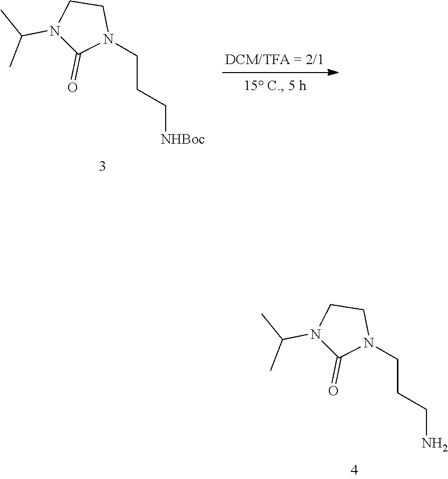 [SMILES: CC(C)N1CCN(CCCNC(=O)OC(C)(C)C)C1=O ; CC(C)N1CCN(CCCN)C1=O]

To a solution of Compound 3 (0.25 g, 876.02 μmol, 1 eq.) in DCM (3 mL) was added TFA (959.38 mg, 8.41 mmol, 625.00 μL, 9.60 eq.) at 15° C. for 5 hours. TLC (Dichloromethane:Methanol=10:1, R_{f}=0.2) showed Compound 3 was consumed and new spots formed. The mixture was concentrated to give Compound 4 (0.26 g, 868.72 μmol, 99.17% yield, TFA) as a yellow oil.

### General Procedure for Preparation of Compound 6:

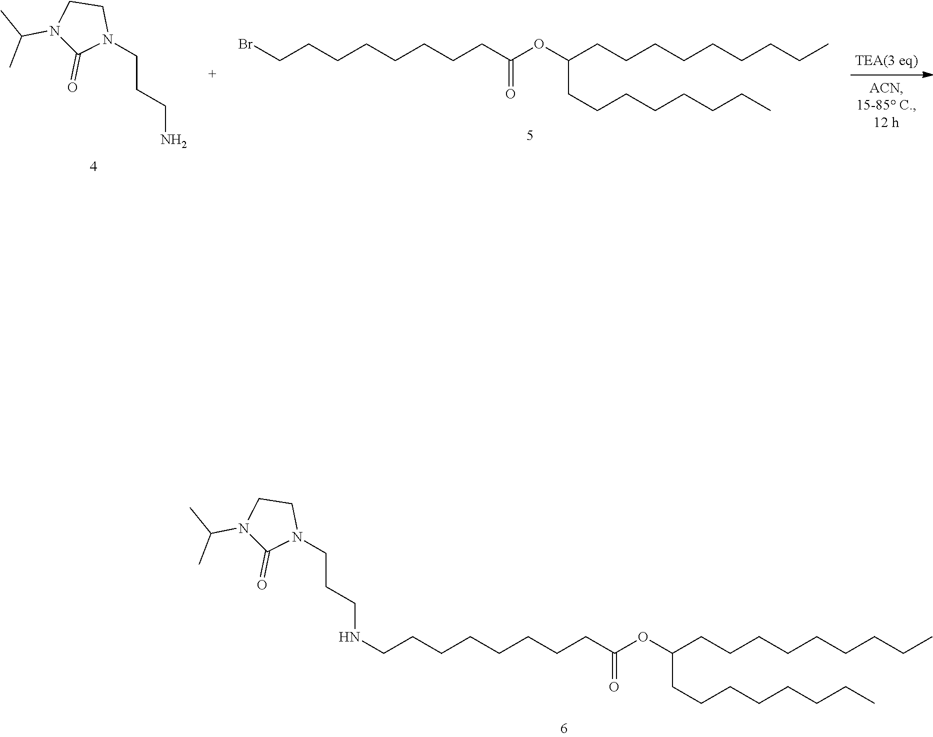

To a solution of Compound 4 (210.01 mg, 1.13 mmol, 1.5 eq.) and Compound 5 (370 mg, 755.70 μmol, 1 eq.) in ACN (1.5 mL) was added TEA (229.41 mg, 2.27 mmol, 315.55 μL, 3 eq.) at 15° C. The reaction mixture was stirred at 85° C. for 12 hours. TLC (Dichloromethane:Methanol=10:1, R_{f}=0.4) showed Compound 5 was consumed and new spots formed. The reaction mixture was concentrated to give a residue. The residue was purified by flash column (SiO₂, Ethyl acetate to Dichloromethane:Methanol=10:1) to give Compound 6 (100 mg, 168.36 μmol, 22.28% yield) as a yellow oil.

### General Procedure for Preparation of Target Compound 100:

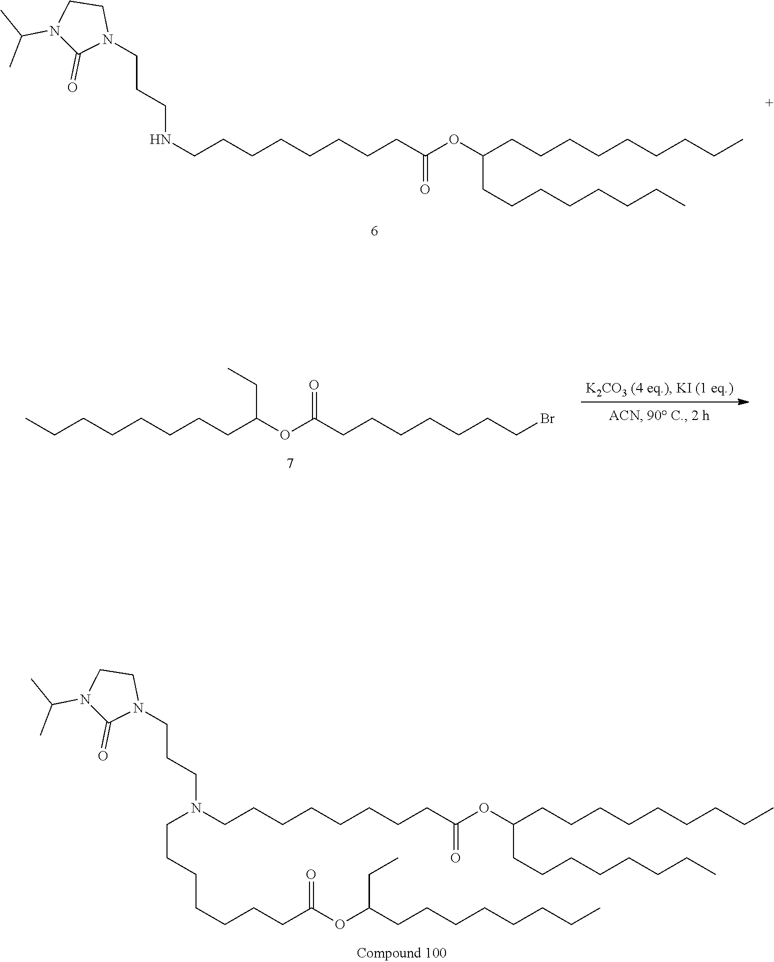

To a solution of Compound 6 (95 mg, 159.94 μmol, 1 eq.) in ACN (1.5 mL) was added K₂CO₃ (88.42 mg, 639.77 μmol, 4 eq.), KI (26.55 mg, 159.94 μmol, 1 eq.) and Compound 7 (60.36 mg, 159.94 μmol, 1 eq.). The mixture was stirred at 90° C. for 2 hours. LCMS showed 2% of Compound 6 remained, and one main peak with the desired mass was detected. The reaction mixture was filtered. The filtrate was diluted with ethyl acetate (10 mL) and washed with H₂O (5 mL×3), dried over Na₂SO₄, filtered and concentrated under reduced pressure to give a residue. The residue was purified by prep-HPLC (column: Xselect CSH C18 100×30 mm×5 um; mobile phase: [H₂O (0.04% HCl)-ACN:THF=1:1]; gradient: 45%-85% B over 12.0 min) to give Compound 100 (27.51 mg, 30.89 μmol, 19.32% yield, 100% purity) as a yellow oil.

LCMS [M+1]⁺=890.8.

¹H NMR (400 MHz, CHLOROFORM-d) δ=12.06 (br s, 1H), 4.92-4.75 (m, 2H), 4.11 (quind, J=6.8, 13.6 Hz, 1H), 3.47-3.38 (m, 2H), 3.35-3.20 (m, 4H), 3.05-2.87 (m, 6H), 2.29 (dt, J=4.8, 7.6 Hz, 4H), 2.15 (br d, J=4.8 Hz, 2H), 1.66-1.49 (m, 14H), 1.39-1.23 (m, 54H), 1.13 (d, J=6.8 Hz, 6H), 0.93-0.82 (m, 12H).

### General Procedure for the Preparation of Compound 101:

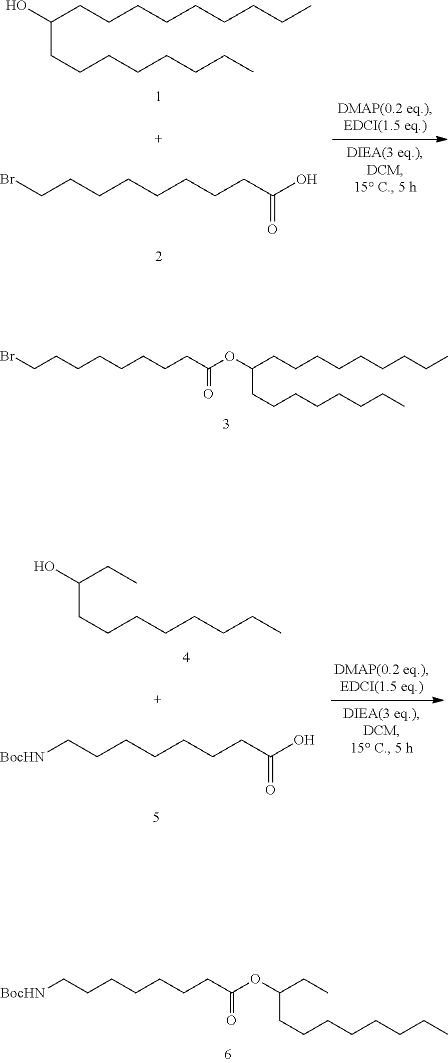 [SMILES: CCCCCCCCCC(O)CCCCCCCC ; O=C(O)CCCCCCCCBr ; CCCCCCCCCC(CCCCCCCC)OC(=O)CCCCCCCCBr ; CCCCCCCCC(O)CC ; CC(C)(C)OC(=O)NCCCCCCCC(=O)O ; CCCCCCCCC(CC)OC(=O)CCCCCCCNC(=O)OC(C)(C)C]

To a solution of Compound 1 (4.56 g, 16.87 mmol, 0.8 eq.) and Compound 2 (5 g, 21.09 mmol, 1 eq.) in DCM (50 mL) was added DMAP (515.18 mg, 4.22 mmol, 0.2 eq.), EDCI (6.06 g, 31.63 mmol, 1.5 eq.) and DIEA (8.18 g, 63.26 mmol, 11.02 mL, 3 eq.). The mixture was stirred at 15° C. for 5 hours. TLC (Petroleum ether:Ethyl acetate=5:1, R_{f}=0.65) indicated Compound 1 was consumed completely and one new spot formed. The reaction mixture was concentrated under reduced pressure to give a residue. The residue was purified by column chromatography (SiO₂, Petroleum ether:Ethyl acetate=60:1) to give Compound 3 (6.9 g, 14.09 mmol, 66.84% yield) as a yellow oil.

In a separate flask chilled in a water bath, Compound 4 and Compound 5 were dissolved in DCM, and DMAP, EDCI and DIEA were added with stirring. TLC was used to track the disappearance of Compound 4, the mixture was concentrated under vacuum, and the residue was purified by column chromatography.

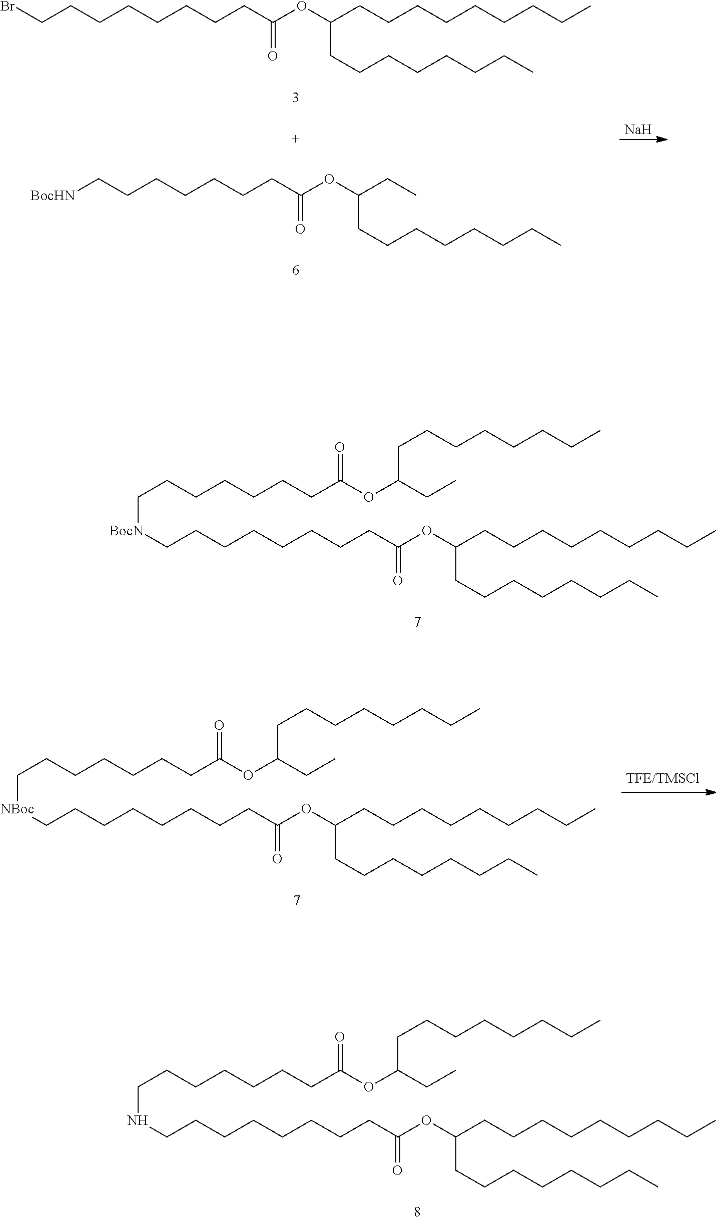

To a solution of Compound 3 in THF was added Compound 6 and sodium hydroxide. TLC showed the disappearance of Compound 1, and the residue was purified by column chromatography to give Compound 7. This compound was dissolved in trifluoroethanol, to which TMSCl was added. The reaction was stirred until TLC showed the quantitative conversion to the free amine. The mixture was concentrated under vacuum and the residue was purified by column chromatography to give Compound 8.

¹H NMR (400 MHz, CHLOROFORM-d) δ=4.87 (quin, J=6.4 Hz, 1H), 3.45-3.38 (m, 2H), 2.29 (t, J=7.6 Hz, 2H), 1.91-1.81 (m, 2H), 1.67-1.58 (m, 2H), 1.51 (br d, J=5.6 Hz, 4H), 1.46-1.39 (m, 2H), 1.34-1.23 (m, 32H), 0.89 (t, J=6.8 Hz, 6H)

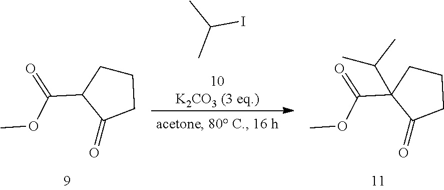

To a solution of Compound 9 (41.81 g, 294.13 mmol, 36.52 mL, 1 eq.) in acetone (420 mL) was added K₂CO₃ (121.96 g, 882.40 mmol, 3 eq.) and Compound 10 (100 g, 588.27 mmol, 58.72 mL, 2 eq.). The mixture was stirred at 80° C. for 16 hours. TLC (Commercial hexanes:Ethyl acetate=5:1, R_{f}=0.53) indicated Compound 9 was consumed completely and many new spots formed. The reaction mixture was diluted with H₂O (500 mL) and extracted with Ethyl acetate (300 mL×2). The combined organic layers were dried over Na₂SO₄, filtered and concentrated under reduced pressure to give crude product. The crude product was treated with HCl (1 M, 10 V) at 20° C. for 1 hour. The mixture was extracted with Ethyl acetate (200 mL*2). The combined organic layers were dried over Na₂SO₄, filtered and concentrated under reduced pressure to give a residue. The residue was purified by column chromatography (SiO₂, Petroleum ether:Ethyl acetate=100:1 to 10:1) to give Compound 11 (22.1 g, 113.00 mmol, 40.04% yield, 94.2% purity) as a colorless oil.

¹H NMR (400 MHz, CHLOROFORM-d) δ=3.90-3.53 (m, 3H), 2.68-2.53 (m, 1H), 2.53-2.32 (m, 2H), 2.23-2.07 (m, 1H), 2.03-1.82 (m, 3H), 0.86 (dd, J=6.8, 14.8 Hz, 6H)

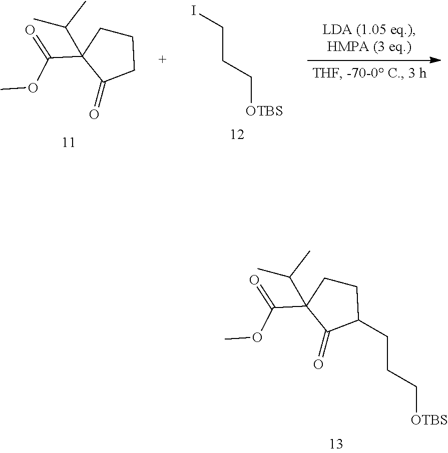 [SMILES: COC(=O)C1(C(C)C)CCCC1=O ; CC(C)(C)[Si](C)(C)OCCCI ; COC(=O)C1(C(C)C)CCC(CCCO[Si](C)(C)C(C)(C)C)C1=O]

To a solution of Compound 11 (7.5 g, 40.71 mmol, 1 eq.) in THF (225 mL) was added LDA (2 M, 21.37 mL, 1.05 eq.) at −70° C. The mixture was stirred for 30 mins at the same temperature and for 30 mins at 0° C. HMPA (21.89 g, 122.13 mmol, 21.37 mL, 3 eq.) was added to the mixture at the same temperature and the mixture was re-cooled to −70° C. Compound 12 (12.83 g, 42.75 mmol, 1.05 eq.) was added to the mixture at the same temperature, and the mixture was stirred at 0° C. for 3 hours. TLC (Commercial hexanes:Ethyl acetate=8:1, R_{f}=0.6&0.65) indicated Compound 11 remained, and two new spots with desired compound was detected. The reaction mixture was diluted with Sat. NH₄Cl (75 mL) and extracted with ethyl acetate (75 mL×3). The combined organic layers were washed with brine (75 mL), dried over Na₂SO₄, filtered and concentrated to give a residue. The residue was purified by column chromatography (SiO₂, Petroleum ether:Ethyl acetate=50:1 to 5:1) to give Compound 13 (6.8 g, 9.73 mmol) as a yellow oil.

¹H NMR (400 MHz, CHLOROFORM-d) δ=3.71-3.66 (m, 3H), 3.64-3.57 (m, 2H), 2.67-2.35 (m, 3H), 2.26-2.08 (m, 2H), 2.01-1.72 (m, 3H), 1.62 (br dd, J=6.8, 12.0 Hz, 1H), 1.53 (br d, J=8.4 Hz, 1H), 1.38-1.28 (m, 1H), 0.89 (s, 9H), 0.87-0.79 (m, 6H), 0.08-0.00 (m, 6H)

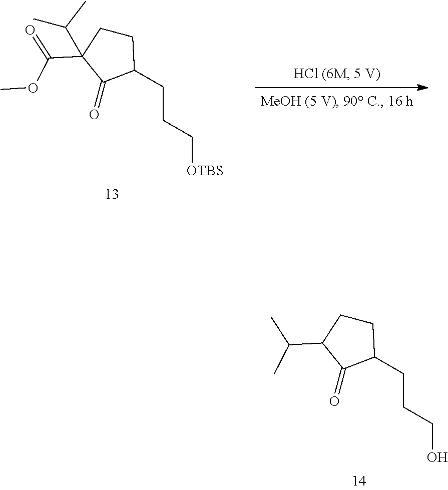 [SMILES: COC(=O)C1(C(C)C)CCC(CCCO[Si](C)(C)C(C)(C)C)C1=O ; CC(C)C1CCC(CCCO)C1=O]

A solution of Compound 13 (3.2 g, 8.97 mmol, 1 eq.) in HCl (6M, 16 mL) and MeOH (16.00 mL) was stirred at 90° C. for 16 hours. TLC (Commercial hexanes:Ethyl acetate=1:1, R_{f}=0.24) indicated Compound 13 was consumed completely and many new spots formed. The reaction mixture was quenched by addition Sat. NH₄Cl (50 mL) at 20° C., and then extracted with Ethyl acetate (50 mL×3). The combined organic layers were washed with brine (50 mL*2), dried over Na₂SO₄, filtered and concentrated under reduced pressure to give a residue. The residue was purified by column chromatography (SiO₂, Petroleum ether:Ethyl acetate=80:1 to 20:1) to give Compound 13 (1.15 g, 6.24 mmol) as a yellow oil.

¹H NMR (400 MHz, DMSO-d6) δ=4.36 (t, J=5.2 Hz, 1H), 3.45-3.33 (m, 2H), 2.20-1.80 (m, 5H), 1.77-1.63 (m, 1H), 1.60-1.38 (m, 3H), 1.38-1.25 (m, 1H), 1.25-1.10 (m, 1H), 0.98-0.86 (m, 3H), 0.82-0.66 (m, 3H)

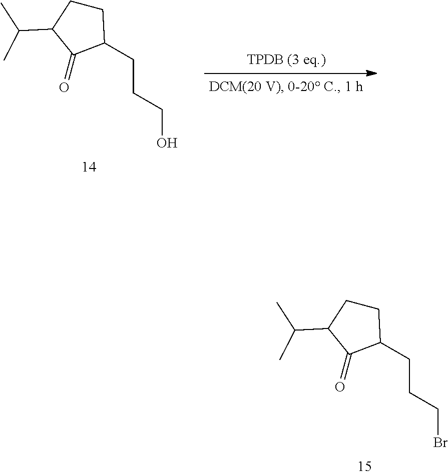

To a solution of Compound 14 (200 mg, 1.09 mmol, 1 eq.) in DCM (2 mL) was added TPDB (1.37 g, 3.26 mmol, 3 eq.) at 0° C. The mixture was stirred at 20° C. for 1 hour. TLC (Commercial hexanes:Ethyl acetate=5:1, R_{f}=0.60) indicated Compound 14 was consumed completely and many new spots formed. The reaction mixture was filtered and concentrated under reduced pressure to give a residue. The residue was diluted with Ethyl acetate (2 mL), filtered and filtrate was concentrated under reduced pressure to give a residue. The residue was purified by column chromatography (SiO₂, Petroleum ether:Ethyl acetate=80:1 to 20:1) to give Compound 15 (235 mg, 950.76 μmol) as a yellow oil.

¹H NMR (400 MHz, DMSO-d6) δ=3.53 (t, J=6.4 Hz, 2H), 2.31-1.58 (m, 8H), 1.57-1.42 (m, 1H), 1.39-1.19 (m, 2H), 0.99-0.87 (m, 3H), 0.81-0.66 (m, 3H)

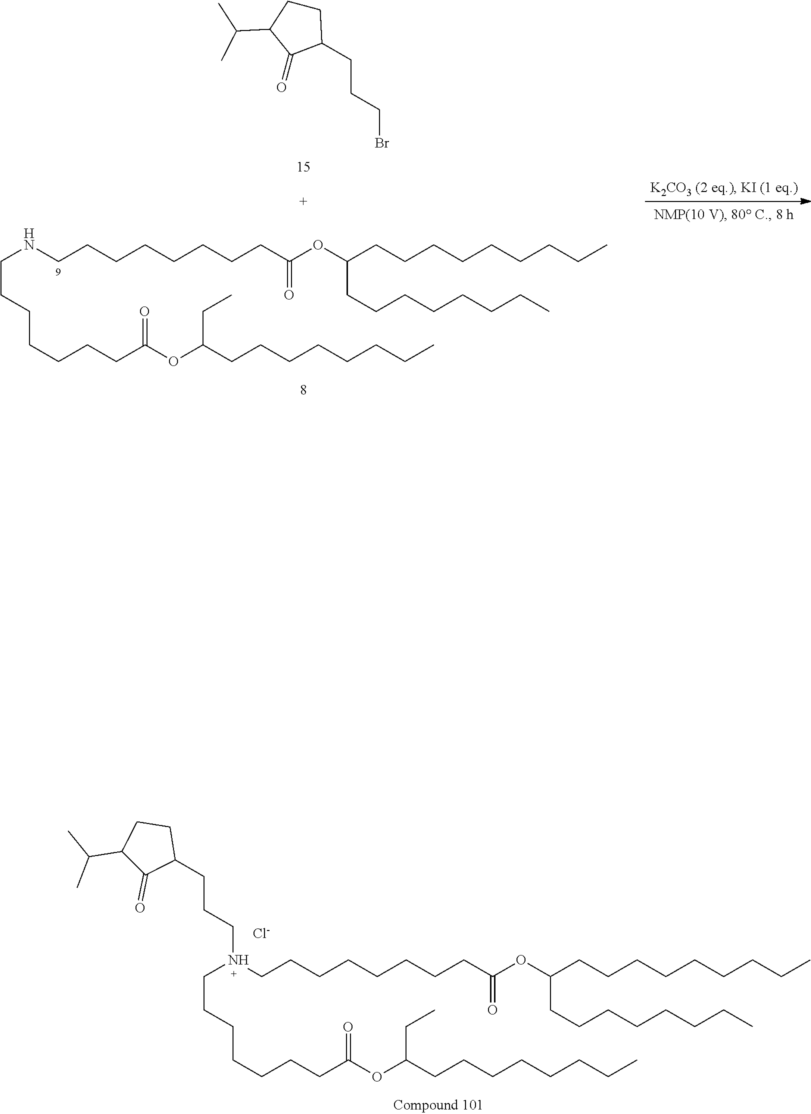

To a solution of Compound 15 (80 mg, 323.66 μmol, 0.8 eq.) in NMP (1 mL) was added K₂CO₃ (111.83 mg, 809.16 μmol, 2 eq.), NaI (60.64 mg, 404.58 μmol, 1 eq.) and Compound 8 (292.19 mg, 404.58 μmol, 1 eq.) at 20° C. The mixture was stirred at 80° C. for 8 hours. LCMS showed Compound 8 was consumed completely and one main peak with desired MS was detected. The reaction mixture was diluted with H₂O (3 mL) and extracted with Ethyl acetate (3 mL×3). The combined organic layers were dried over Na₂SO₄, filtered and concentrated under reduced pressure to give a residue. The residue was purified by column chromatography (SiO₂, Petroleum ether:Ethyl acetate=80:1 to 20:1) to give crude product. The crude product was re-purified by prep-HPLC (column: Welchrom CSH C18 100×30×7; mobile phase: [H₂O (0.02% HCl)-ACN:THF=1:1]; gradient: 55%-95% B over 14.0 min) to give Compound 101 (188 mg, 203.26 μmol, 50.24% yield, HCl) as a light yellow oil. HRMS-TOF [M+1]⁺=888.8

¹H NMR (400 MHz, CHLOROFORM-d) δ=12.41-11.93 (m, 1H), 4.95-4.74 (m, 2H), 3.10-2.83 (m, 6H), 2.33-2.25 (m, 4H), 2.24-2.08 (m, 2H), 2.06-1.90 (m, 4H), 1.88-1.71 (m, 5H), 1.69-1.58 (m, 6H), 1.53-1.46 (m, 6H), 1.45-1.17 (m, 56H), 1.02-0.95 (m, 3H), 0.94-0.85 (m, 12H), 0.84-0.77 (m, 3H)

### General Procedure for the Preparation of Compound 102:

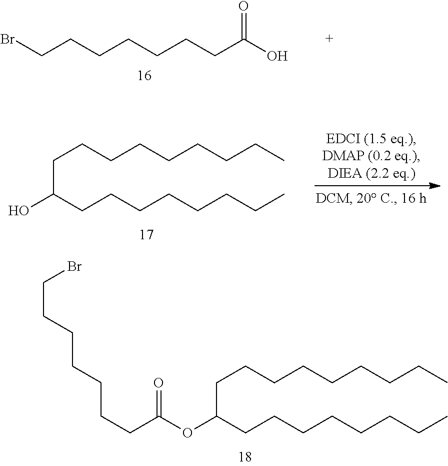 [SMILES: O=C(O)CCCCCCCBr ; CCCCCCCCCC(O)CCCCCCCC ; CCCCCCCCCC(CCCCCCCC)OC(=O)CCCCCCCBr]

To a solution of Compound 16 (2.14 g, 9.61 mmol, 1.30 eq.) in DCM (20 mL) was added DMAP (181 mg, 1.48 mmol, 0.20 eq.), EDCI (2.13 g, 11.1 mmol, 1.50 eq.), DIEA (2.20 g, 17.0 mmol, 2.96 mL, 2.30 eq.) and Compound 17 (2.00 g, 7.39 mmol, 1.00 eq.). The mixture was stirred at 20° C. for 16 hours. TLC (commercial hexanes:ethyl acetate=10:1, R_{f}=0.7) indicated Compound 16 was consumed completely and one new spot formed. The reaction mixture was concentrated under reduced pressure to give a residue. The residue was purified by column chromatography (SiO₂, Petroleum ether:Ethyl acetate=97:3 to 95:5) to give Compound 8 (2.60 g, 5.47 mmol, 73.9% yield) as a light-yellow oil.

LCMS [M+23]⁺=497.3

¹H NMR (400 MHz, CDCl3) δ=4.87 (t, J=6.4 Hz, 1H), 3.41 (t, J=6.8 Hz, 2H), 2.29 (t, J=7.2 Hz, 2H), 1.86 (quin, J=7.2 Hz, 2H), 1.69-1.58 (m, 2H), 1.51 (br d, J=5.6 Hz, 4H), 1.47-1.39 (m, 2H), 1.37-1.18 (m, 30H), 0.89 (t, J=6.8 Hz, 6H)

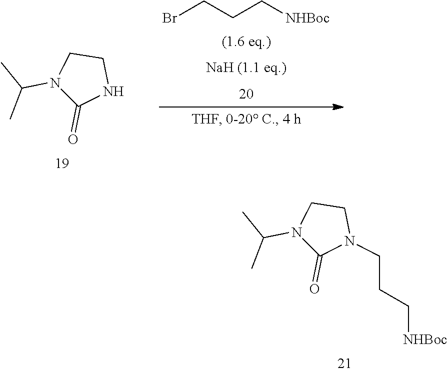

To a solution of Compound 19 (2.00 g, 15.6 mmol, 1 eq.) in THF (40 mL) was added NaH (687 mg, 17.2 mmol, 60% purity, 1.1 eq.) at 0° C. for 0.5 hour. Then, Compound 20 (5.95 g, 25.0 mmol, 1.6 eq.) was added at 0° C. The resulting mixture was stirred at 20° C. for 3.5 hours. TLC (Petroleum ether:Ethyl acetate=0:1, R_{f}=0.5) indicated Compound 19 remained and new spots formed. The reaction mixture was quenched by addition of saturated NH₄Cl (50 mL) at 0° C., and then extracted with ethyl acetate (50 mL×2), dried over Na₂SO₄, filtered and concentrated under reduced pressure to give a residue. The residue was purified by column chromatography (SiO₂, Petroleum ether:Ethyl acetate=80:20 to 39:61) to give Compound 21 (2.50 g, 7.97 mmol, 51.1% yield, 91.0% purity) as a light-yellow oil.

LCMS [M+1]⁺=286.2

¹H NMR (400 MHz, CDCl₃) δ=5.33 (br s, 1H), 4.21-4.03 (m, 1H), 3.34-3.21 (m, 6H), 3.12 (br d, J=1.2 Hz, 2H), 1.65 (t, J=6.4 Hz, 2H), 1.44 (s, 9H), 1.12 (d, J=6.8 Hz, 6H)

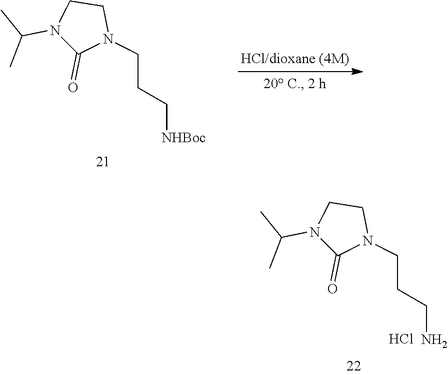 [SMILES: CC(C)N1CCN(CCCNC(=O)OC(C)(C)C)C1=O ; CC(C)N1CCN(CCCN)C1=O ; Cl]

A mixture of Compound 21 (2.50 g, 8.76 mmol, 1.00 eq.) in HCl/dioxane (12 mL) was degassed and purged with N₂ three times, and the mixture was stirred at 20° C. for 2 hours under an N₂ atmosphere. TLC (DCM:MeOH=20:1, R_{f}=0.17) indicated Compound 21 was consumed completely and new spots formed. The reaction mixture was concentrated and lyophilized to give Compound 22 (2.20 g, crude, HCl) as a brown solid.

LCMS [M+1]⁺=186.2

¹H NMR (400 MHz, CDCl3) δ=8.39 (br s, 3H), 4.09 (quin, J=6.8 Hz, 1H), 3.59-3.28 (m, 6H), 3.11 (br d, J=5.2 Hz, 2H), 2.08 (br t, J=6.0 Hz, 2H), 1.14 (d, J=6.8 Hz, 6H)

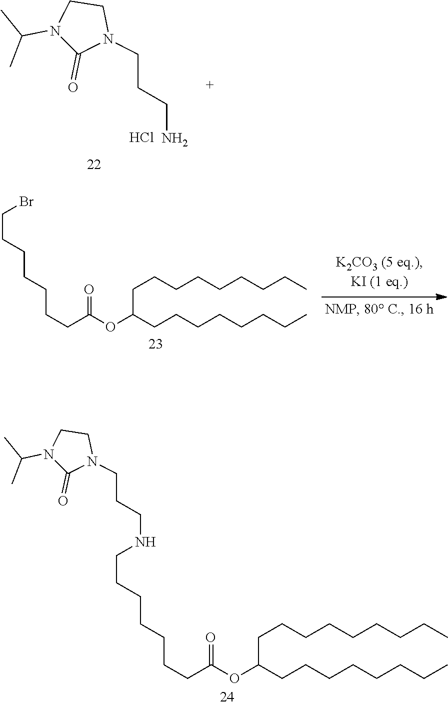

To a solution of Compound 22 (2.20 g, 9.92 mmol, 5 eq., HCl) in NMP (10 mL) was added Compound 23 (944 mg, 1.98 mmol, 1 eq.), K₂CO₃ (1.37 g, 9.92 mmol, 5 eq.) and KI (329 mg, 1.98 mmol, 1 eq.). The mixture was stirred at 80° C. for 16 hours. TLC indicated Compound 22 was consumed completely and many new spots formed. The mixture was filtered through a Celite pad, and the filtrate was diluted with ethyl acetate (50 mL). The combined organic layers were washed with brine (30 mL×5), dried over Na₂SO₄, filtered and concentrated under reduced pressure to give a residue. The residue was purified by column chromatography (SiO₂, DCM:MeOH=94:6 to 88:12) to give Compound 24 (250 mg, 366 μmol, 18.5% yield, 85.0% purity) as a light yellow oil.

LCMS [M+1]⁺=580.5

¹H NMR (400 MHz, CDCl₃) δ=4.86 (br t, J=6.0 Hz, 1H), 4.20-4.00 (m, 1H), 3.41-3.25 (m, 6H), 3.18 (br s, 1H), 2.89-2.73 (m, 3H), 2.28 (t, J=7.6 Hz, 2H), 2.08-1.90 (m, 2H), 1.73 (br s, 2H), 1.67-1.57 (m, 2H), 1.51 (br d, J=5.2 Hz, 4H), 1.43-1.16 (m, 32H), 1.15-1.07 (m, 6H), 0.88 (t, J=6.8 Hz, 6H)

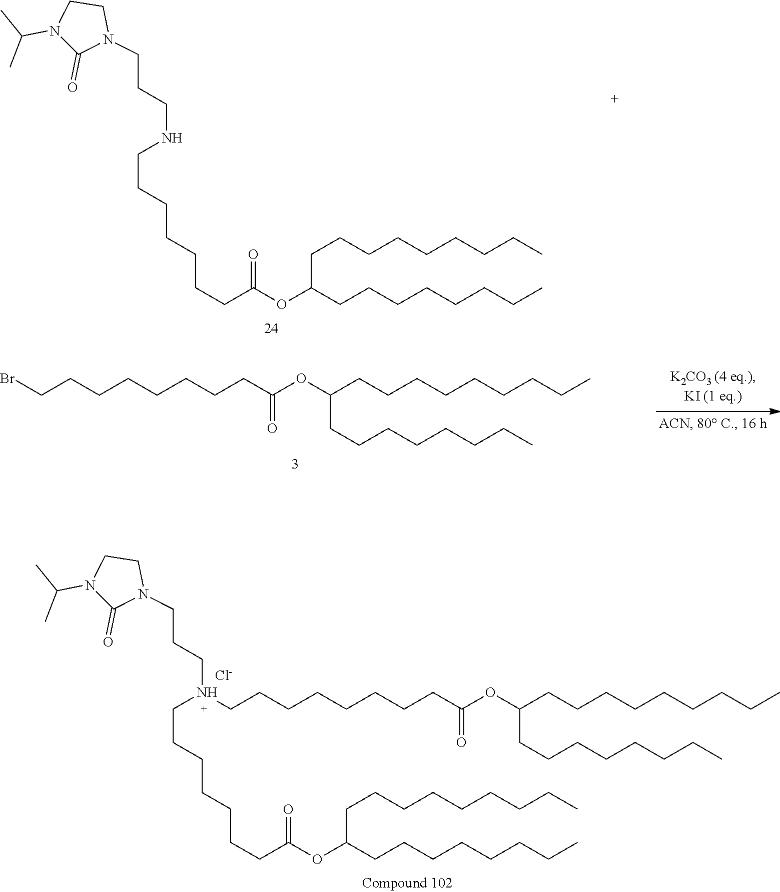

To a solution of Compound 24 (450 mg, 776 μmol, 1 eq.) in ACN (6 mL) was added Compound 8 (418 mg, 854 μmol, 1.1 eq.), KI (129 mg, 776 μmol, 1 eq.) and K₂CO₃ (429 mg, 3.10 mmol, 4 eq.). The mixture was stirred at 90° C. for 2 hours. TLC (Dichloromethane:Methanol=10:1, R_{f}=0.5) indicated Compound 24 was consumed completely and one new spot formed. The mixture was diluted with ethyl acetate (50 mL) and washed with H₂O (30 mL×3), dried over Na₂SO₄, filtered and concentrated under reduced pressure to give a residue. The residue was purified by column chromatography (SiO₂, commercial hexanes:ethyl acetate=50:50 to 0:1) to give crude product. The crude product was purified by prep-HPLC (Welchoursom CSH C18 100×30×7; mobile phase: [H₂O (0.02% HCl)-ACN:THF=1:1]; gradient: 60%-90% B over 16.0 min) to give Compound 102 (400 mg, 387 μmol, 49.9% yield, 99.6% purity, 1.1 HCl) as a light yellow oil.

LCMS [M+1]⁺=988.7

¹H NMR (400 MHz, CDCl₃) δ=12.06 (br s, 1H), 4.87 (quin, J=6.4 Hz, 2H), 4.11 (dt, J=13.6, 6.8 Hz, 1H), 3.47-3.36 (m, 2H), 3.34-3.19 (m, 4H), 3.06-2.86 (m, 6H), 2.28 (t, J=7.2 Hz, 4H), 2.22-2.09 (m, 2H), 1.76 (br d, J=4.0 Hz, 4H), 1.69-1.57 (m, 6H), 1.51 (br d, J=5.6 Hz, 8H), 1.38-1.23 (m, 66H), 1.13 (d, J=6.8 Hz, 6H), 0.89 (t, J=6.8 Hz, 12H)

### General Procedure for the Preparation of Compound 103:

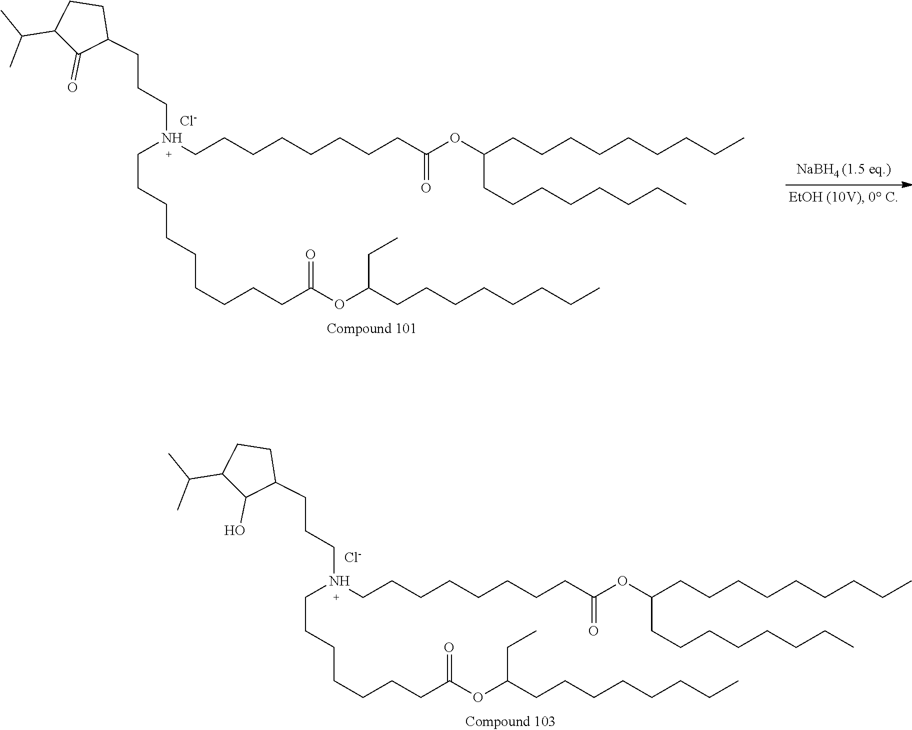

To a solution of Compound 101 (50 mg, 56.28 μmol, 1 eq.) in EtOH (0.5 mL) was added NaBH₄ (3.19 mg, 84.41 μmol, 1.5 eq.) at 0° C. The mixture was stirred at 20° C. for 2 hours. TLC (Commercial hexanes:Ethyl acetate=3:1, R_{f}=0.36) indicated Compound 101 was consumed completely and one new spot formed. The reaction mixture was quenched by addition H₂O (5 mL) at 20° C., and then extracted with DCM (3 mL*3). The combined organic layers were concentrated under reduced pressure to give a residue. The residue was purified by column chromatography (SiO2, Commercial hexanes:Ethyl acetate=10:1 to 5:1) to give Compound 103 (Free) (135 mg, 151.60 μmol) as light yellow oil.

¹H NMR (400 MHz, CHLOROFORM-d) δ=4.96-4.74 (m, 2H), 3.87 (br s, 1H), 2.48-2.34 (m, 5H), 2.29 (dt, J=4.4, 7.6 Hz, 4H), 2.06-1.93 (m, 1H), 1.89-1.73 (m, 2H), 1.73-1.56 (m, 8H), 1.52 (br dd, J=4.0, 6.4 Hz, 8H), 1.48-1.37 (m, 8H), 1.36-1.19 (m, 54H), 1.03-0.97 (m, 3H), 0.94 (d, J=6.4 Hz, 3H), 0.91-0.85 (m, 12H)

A mixture of TFE (73.02 mg, 729.93 μmol, 52.50 μL, 5 eq.), TMSCl (79.30 mg, 729.93 μmol, 92.64 μL, 5 eq.) was stirred at 20° C. for 1 hour. Then Compound 103 (Free) (130 mg, 145.99 μmol, 1 eq.) in DCM (1.3 mL) was added. The mixture was stirred at 20° C. for 2 hours. The reaction mixture was concentrated under reduced pressure to give Compound 103 (60 mg, 64.00 μmol, 43.84% yield, 98.87% purity, HCl) as a light-yellow oil.

HRMS-TOF [M+1]⁺=890.8

¹H NMR (400 MHz, CHLOROFORM-d) δ=12.11-11.79 (m, 1H), 4.94-4.74 (m, 2H), 4.02-3.83 (m, 1H), 3.17-2.77 (m, 6H), 2.37-2.23 (m, 4H), 2.06-1.92 (m, 2H), 1.91-1.68 (m, 8H), 1.67-1.58 (m, 6H), 1.56-1.47 (m, 8H), 1.45-1.17 (m, 56H), 1.03-0.97 (m, 3H), 0.94 (d, J=6.4 Hz, 3H), 0.91-0.85 (m, 12H)

### General Procedure for the Preparation of Compound 104:

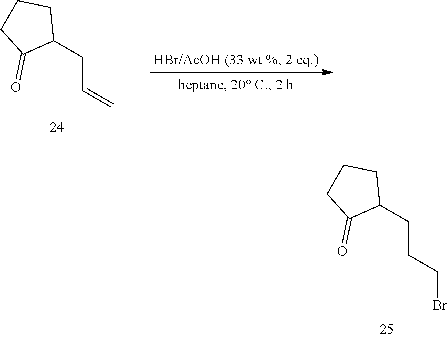

To a solution of Compound 24 (600 mg, 4.83 mmol, 1 eq.) in HEPTANE (6 mL) was added HBr (2.37 g, 9.66 mmol, 1.59 mL, 2 eq.). The mixture was stirred at 20° C. for 2 hours. TLC (commercial hexanes:ethyl acetate=3:1, R_{f}=0.26) showed Compound 24 was consumed completely and new spot formed. The reaction mixture was basified with Sat. NaHCO₃ (20 ml) adjust pH=7-8 and extracted with DCM (20 mL×3). The combined organic layers were concentrated under reduced pressure to give Compound 25 (790 mg, crude) as a colorless oil.

LCMS [M+1]⁺=205.2

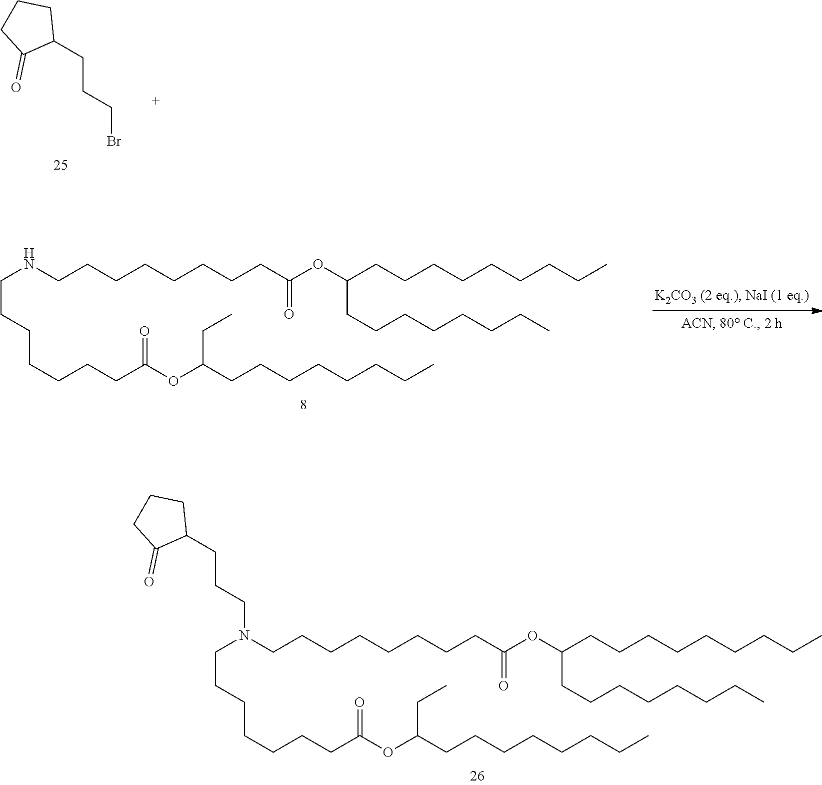 [SMILES: O=C1CCCC1CCCBr ; CCCCCCCCCC(CCCCCCCC)OC(=O)CCCCCCCCNCCCCCCCC(=O)OC(CC)CCCCCCCC ; CCCCCCCCCC(CCCCCCCC)OC(=O)CCCCCCCCN(CCCCCCCC(=O)OC(CC)CCCCCCCC)CCCC1CCCC1=O]

To a solution of Compound 25 (790 mg, 3.85 mmol, 2 eq.) in ACN (13.9 mL) was added Compound 8 (1.39 g, 1.93 mmol, 1 eq.), K₂CO₃ (532.36 mg, 3.85 mmol, 2 eq.) and NaI (288.69 mg, 1.93 mmol, 1 eq.). The mixture was stirred at 80° C. for 2 hours. LCMS showed Compound 28 was consumed completely, and the desired MS was detected. The reaction mixture was filtered and concentrated under reduced pressure to give a residue. The residue was purified by column chromatography (SiO₂, Commercial hexanes:Ethyl acetate=20:1 to 5:1) to give Compound 26 as a light yellow oil.

LCMS [M+1]⁺=846.8

¹H NMR (400 MHz, CHLOROFORM-d) δ=4.93-4.77 (m, 2H), 2.46-2.18 (m, 12H), 2.16-1.96 (m, 3H), 1.86-1.65 (m, 4H), 1.65-1.37 (m, 20H), 1.33-1.23 (m, 50H), 0.93-0.85 (m, 12H)

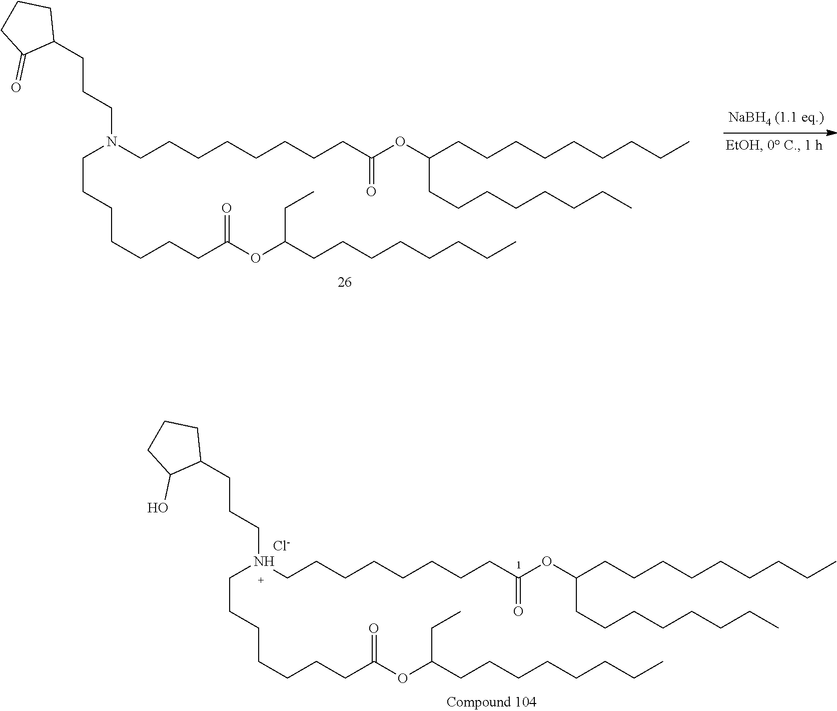 [SMILES: CCCCCCCCCC(CCCCCCCC)OC(=O)CCCCCCCCN(CCCCCCCC(=O)OC(CC)CCCCCCCC)CCCC1CCCC1=O ; CCCCCCCCCC(CCCCCCCC)OC(=O)CCCCCCCCN(CCCCCCCC(=O)OC(CC)CCCCCCCC)CCCC1CCCC1O ; [Cl-]]

To a solution of Compound 26 (560 mg, 661.63 μmol, 1 eq.) in EtOH (0.56 mL) was added NaBH₄ (27.53 mg, 727.79 μmol, 1.1 eq.) at 0° C. The resulting mixture was stirred at 0° C. for 1 hour. TLC (Commercial hexanes:Ethyl acetate=2:1, R_{f}=0.28) showed Compound 26 was consumed completely and new spot formed. The reaction mixture was quenched by addition H₂O (20 mL) at 20° C., and extracted with DCM (20 mL×3). The combined organic layers were concentrated under reduced pressure to give a residue. The residue was purified by column chromatography (SiO₂, Commercial hexanes:Ethyl acetate=20:1 to 10:1) to give crude product. The crude product was purified by prep-HPLC (column: Welchrom CSH C18 100×30×7; mobile phase: [H₂O (0.02% HCl)-ACN:THF=1:1]; gradient: 45%-95% B over 14.0 min) to give Compound 104 (240 mg, 282.88 μmol, 42.76% yield) as a colorless oil.

LCMS [M+1]⁺=848.8

¹H NMR (400 MHz, CHLOROFORM-d) δ=11.98-11.79 (m, 1H), 4.91-4.77 (m, 2H), 3.86 (q, J=6.0 Hz, 1H), 3.16-2.86 (m, 6H), 2.35-2.22 (m, 4H), 2.07-1.88 (m, 3H), 1.84-1.80 (m, 2H), 1.71 (br d, J=8.0 Hz, 2H), 1.68-1.44 (m, 16H), 1.43-1.06 (m, 56H), 0.88 (br t, J=6.4 Hz, 12H)

### General Procedure for the Preparation of Compound 105:

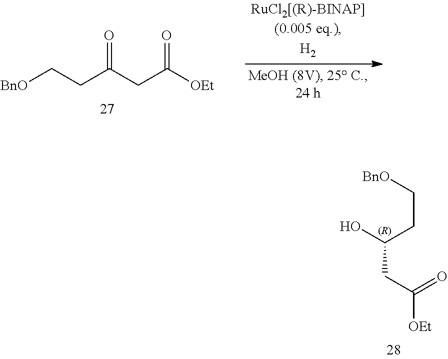

RuCl₂[(R)-BINAP](45.69 mg, 59.93 μmol, 0.005 eq.) was added to a degassed solution of Compound 27 (3 g, 11.99 mmol, 1 eq.) in EtOH (24 mL). The mixture was transferred to an autoclave, which was flushed with argon for several min and then purged three times with H₂. The mixture was stirred 24 hours at 25° C. under an H₂ atmosphere (3 MPa). TLC (Commercial hexanes:Ethyl acetate=4:1, R_{f}=0.33) indicated that Compound 27 was consumed completely and new spots formed. The reaction mixture was concentrated under reduced pressure to give a residue (2.96 g). The residue was purified by flash silica gel chromatography (ISCO®; 80 g SepaFlash® Silica Flash Column, Eluent of 6 to 9 to 12% Ethyl acetate/Commercial hexanes gradient @100 mL/min) to give the product (1.68 g, light yellow oil) and Compound 27 (1 g). The residue (2.96 g) was purified by flash silica gel chromatography (ISCO®; 80 g SepaFlash® Silica Flash Column, Eluent of 6 to 9 to 12% Ethyl acetate/Commercial hexanes gradient @100 mL/min) to give the product (1.82 g, light yellow oil) and the product (970 mg). The total product Compound 28 (4.47 g) was obtained as a light-yellow oil.

LCMS [M+1]⁺=253.3

¹H NMR (400 MHz, CHLOROFORM-d) δ=7.42-7.26 (m, 5H), 4.53 (s, 2H), 4.25 (br dd, J=5.2, 6.4 Hz, 1H), 4.17 (q, J=7.2 Hz, 2H), 3.79-3.58 (m, 2H), 3.37 (br s, 1H), 2.50 (d, J=6.4 Hz, 2H), 1.90-1.72 (m, 2H), 1.28 (t, J=7.2 Hz, 3H)

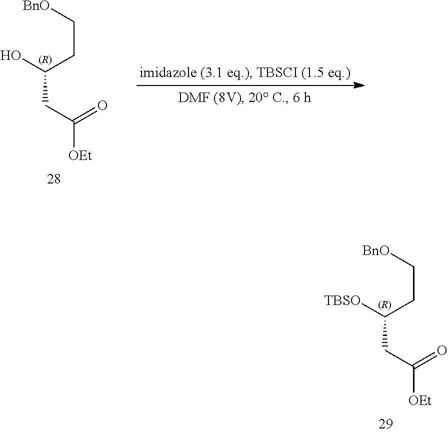

Compound 28 (3.4 g, 13.48 mmol, 1 eq.) and imidazole (2.84 g, 41.77 mmol, 3.1 eq.) was dissolved in DMF (17 mL). Upon cooling to 0° C., TBSCl (3.05 g, 20.21 mmol, 2.49 mL, 1.5 eq.) was added. The mixture was warmed to 20° C. and stirred at 20° C. for 6 hours. TLC (Commercial hexanes:Ethyl acetate=4:1, R_{f}=0.79) indicated Compound 28 was consumed and new spots formed. The combined reaction mixture was poured into a mixture of H₂O (60 mL) and Sat. NaHCO₃ (50 mL). The resultant mixture was extracted with ethyl acetate (50 mL×3). The combined organic layers were washed with brine (50 mL×2), dried over Na₂SO₄, filtered and concentrated under reduced pressure to give a residue (7.26 g). The residue was purified by flash silica gel chromatography (ISCO®; 220 g SepaFlash® Silica Flash Column, Eluent of 0-5% Ethyl acetate/Commercial hexanes gradient @120 mL/min) to give Compound 29 (5.66 g, 82.73% purity) as a colorless oil.

LCMS [M+1]⁺=367.3

¹H NMR (400 MHz, CHLOROFORM-d) δ=7.39-7.27 (m, 3H), 4.54-4.49 (m, 1H), 4.49-4.44 (m, 1H), 4.32 (quin, J=6.0 Hz, 1H), 4.19-4.05 (m, 2H), 3.56 (t, J=6.4 Hz, 2H), 2.48 (d, J=6.2 Hz, 2H), 1.85 (q, J=6.2 Hz, 2H), 1.26 (t, J=7.2 Hz, 3H), 0.87 (s, 9H), 0.06 (s, 3H), 0.05 (s, 3H)

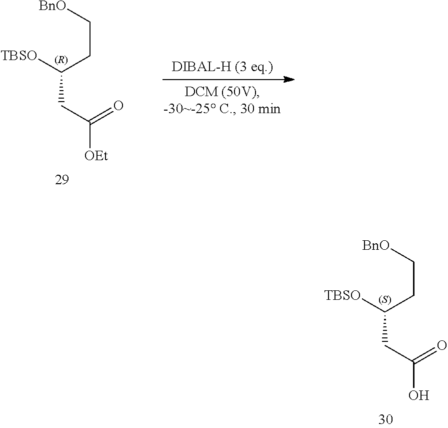

Compound 29 (5.6 g, 15.28 mmol, 1 eq.) was dissolved in DCM (280 mL). Upon cooling to −30° C., DIBAL-H (1 M, 45.83 mL, 3 eq.) was added dropwise via pressure-equalizing dropping funnel. The solution was warmed to −25° C. and stirred for 30 min. TLC (Commercial hexanes:Ethyl acetate=4:1, R_{f}=0.21) indicated that Compound 29 was consumed and new spots formed. The reaction mixture was quenched by a mixture of saturated NH₄Cl (85 mL) and saturated sodium potassium tartrate (255 mL) at −25° C. The resultant mixture was warmed to 20° C. and stirred for 30 min. The mixture was extracted with ethyl acetate (80 mL×2). The combined organic layers were dried over Na₂SO₄, filtered and concentrated under reduced pressure to give a residue. The residue was purified by flash silica gel chromatography (ISCO®; 120 g SepaFlash® Silica Flash Column, Eluent of 5-13% Ethyl acetate/Commercial hexanes gradient @120 mL/min) to give Compound 30 (3 g, 8.29 mmol, 54.29% yield, 89.72% purity) as a colorless oil.

LCMS [M+1]⁺=325.3

¹H NMR (400 MHz, CHLOROFORM-d) δ=7.40-7.27 (m, 5H), 4.56-4.50 (m, 1H), 4.50-4.43 (m, 1H), 4.17-4.07 (m, 1H), 3.90-3.79 (m, 1H), 3.72 (td, J=5.6, 10.8 Hz, 1H), 3.54 (t, J=6.4 Hz, 2H), 2.35 (br s, 1H), 1.95-1.78 (m, 3H), 1.74-1.62 (m, 1H), 0.90 (s, 9H), 0.11 (s, 3H), 0.09 (s, 3H)

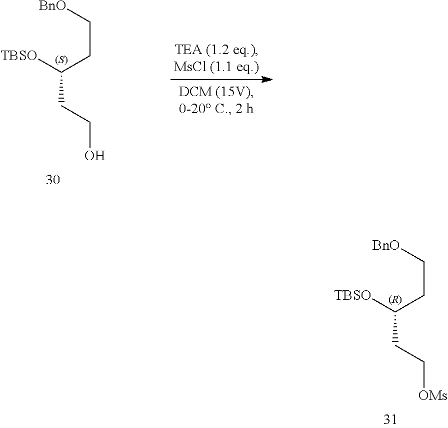

To a solution of Compound 30 (2.4 g, 7.40 mmol, 1 eq.) in DCM (36 mL) at 0° C. was added TEA (897.99 mg, 8.87 mmol, 1.24 mL, 1.2 eq.) and MsCl (931.86 mg, 8.13 mmol, 629.63 μL, 1.1 eq.). The mixture was stirred at 20° C. for 2 hours. TLC (Commercial hexanes:Ethyl acetate=4:1, R_{f}=0.55) indicated Compound 30 was consumed and one new spot formed. The reaction mixture was diluted with DCM (20 mL) and washed with Sat, NaHCO₃ (20 mL). The aqueous layer was extracted with DCM (40 mL×2), and the combined organic layers were washed with brine (40 mL), dried over Na₂SO₄, filtered and evaporated to give Compound 31 (2.9 g, crude) as a colorless oil.

LCMS [M+1]⁺=403.3

¹H NMR (400 MHz, CHLOROFORM-d) δ=7.41-7.27 (m, 5H), 4.54-4.42 (m, 2H), 4.40-4.26 (m, 2H), 4.04 (quin, J=5.6 Hz, 1H), 3.54 (t, J=6.4 Hz, 2H), 2.97 (s, 3H), 2.04-1.92 (m, 1H), 1.92-1.85 (m, 1H), 1.81 (dq, J=3.2, 6.0 Hz, 2H), 0.89 (s, 9H), 0.08 (d, J=3.6 Hz, 6H)

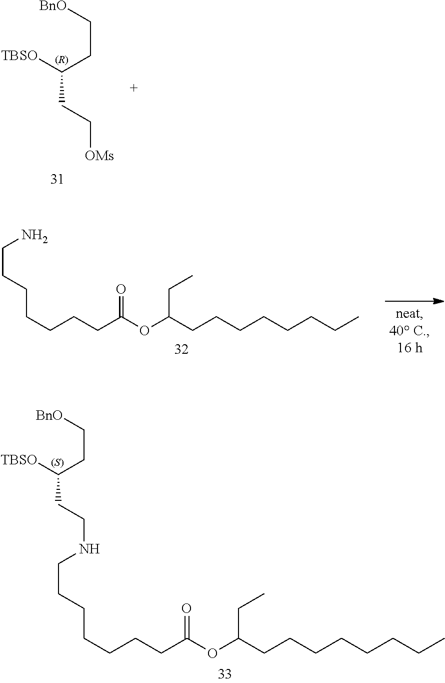

A solution of Compound 31 (2.9 g, 7.20 mmol, 1 eq.) and Compound 32 (10.23 g, 32.63 mmol, 4.53 eq.) was stirred at 40° C. for 16 hours. LCMS showed that ˜58.9% of Compound 32 remained and ˜33% of the desired MS was detected. TLC (Dichloromethane:Methanol=10:1, R_{f}=0.41) indicated that Compound 32 remained and new spots formed. The reaction mixture was purified by column chromatography (SiO₂, Dichloromethane:Methanol=100:1 to 70:1 to 10:1 to 1:1) to give Compound 33 (3.28 g, 4.41 mmol, 61.16% yield, 83.27% purity) as a light-yellow oil.

LCMS [M+1]⁺=620.6

¹H NMR (400 MHz, CHLOROFORM-d) δ=7.39-7.27 (m, 5H), 4.82 (t, J=6.0 Hz, 1H), 4.57-4.40 (m, 2H), 3.98 (quin, J=5.6 Hz, 1H), 3.53 (t, J=6.4 Hz, 2H), 2.91-2.75 (m, 2H), 2.68 (br t, J=7.2 Hz, 2H), 2.28 (t, J=7.6 Hz, 2H), 1.92-1.83 (m, 1H), 1.79 (q, J=6.2 Hz, 3H), 1.65-1.58 (m, 4H), 1.57-1.45 (m, 4H), 1.37-1.21 (m, 19H), 0.93-0.83 (m, 15H), 0.07 (d, J=6.0 Hz, 6H)

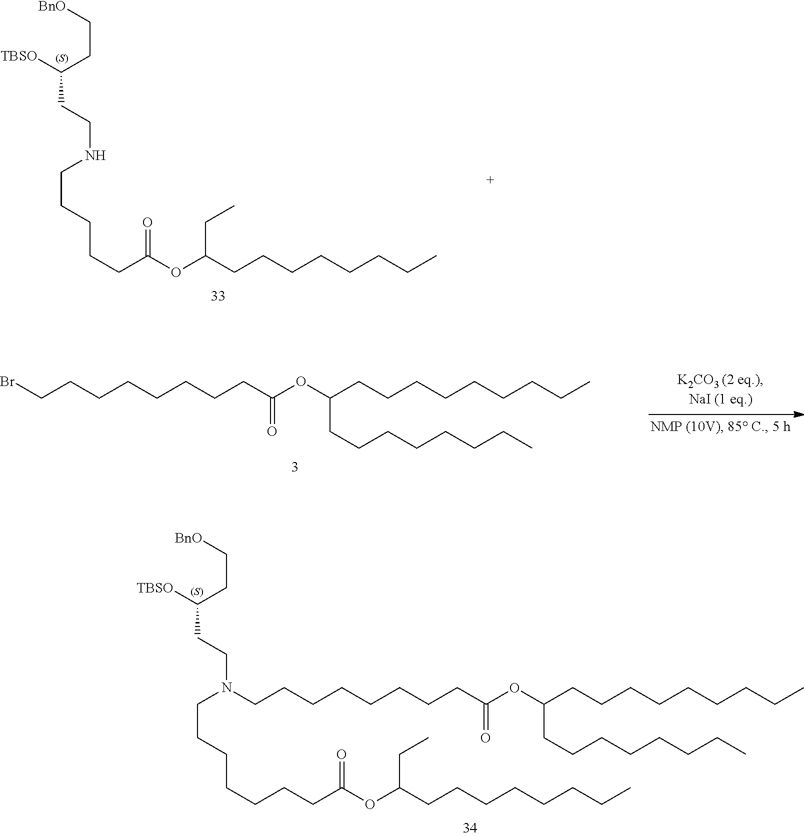

To a solution of Compound 33 (1.39 g, 2.24 mmol, 1 eq.) and Compound 3 (1.10 g, 2.24 mmol, 1 eq.) in NMP (14 mL) was added K₂CO₃ (619.66 mg, 4.48 mmol, 2 eq.) and NaI (336.04 mg, 2.24 mmol, 1 eq.). The mixture was stirred at 85° C. for 5 hours. LCMS showed that ˜7% of Compound 33 remained and ˜28% of the desired MS was detected. The combined crude reaction mixture was diluted with H₂O (80 mL) and extracted with ethyl acetate (50 mL×3). The combined organic layers were washed with brine (80 mL×2), dried over Na₂SO₄, filtered and concentrated under reduced pressure to give a residue. The residue was purified by column chromatography (SiO₂, Commercial hexanes:Ethyl acetate=100:1 to 20:1 to 15:1 to 1:1) to give Compound 34 (2.1 g, 2.02 mmol, 45.07% yield, 99% purity) as a light-yellow oil.

LCMS [M+1]⁺=1029.1

¹H NMR (400 MHz, CHLOROFORM-d) δ=7.39-7.27 (m, 4H), 4.93-4.76 (m, 2H), 4.57-4.42 (m, 2H), 3.86 (quin, J=5.6 Hz, 1H), 3.54 (t, J=6.8 Hz, 2H), 2.58-2.47 (m, 1H), 2.46-2.39 (m, 1H), 2.39-2.32 (m, 4H), 2.28 (dt, J=3.2, 7.6 Hz, 4H), 1.87-1.71 (m, 2H), 1.65-1.58 (m, 6H), 1.55-1.48 (m, 6H), 1.46-1.37 (m, 4H), 1.36-1.19 (m, 54H), 0.94-0.85 (m, 21H), 0.05 (d, J=4.4 Hz, 6H)

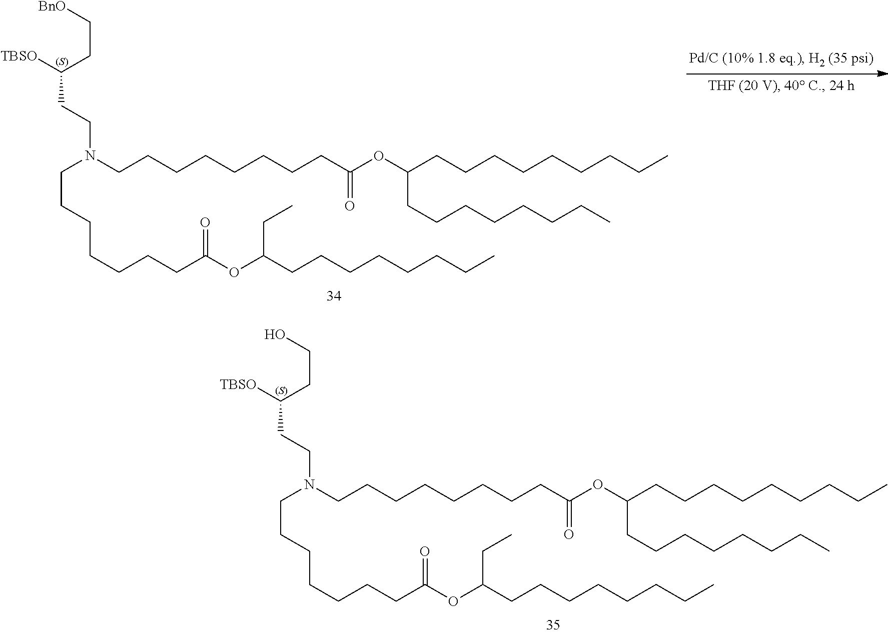

To a solution of Compound 34 (2.35 g, 2.28 mmol, 1 eq.) in THF (58.8 mL) was added Pd/C (4.50 g, 4.23 mmol, 10% purity, 1.85 eq.). The mixture was degassed and purged with H₂ three times, and the mixture was stirred at 40° C. for 16 hours under an H₂ (35 psi) atmosphere. TLC (Commercial hexanes:Ethyl acetate=3:1, R_{f}=0.08, sprayed with ammonia) indicated Compound 34 was consumed completely, and one major new spot with larger polarity was detected. The reaction mixture was filtered through a Celite Pad, and the filtrate was concentrated under reduced pressure to give Compound 35 (2.04 g, crude) as a light-yellow oil.

LCMS [M+1]⁺=939.0

¹H NMR (400 MHz, CHLOROFORM-d) δ=4.94-4.75 (m, 2H), 3.95 (quin, J=5.6 Hz, 1H), 3.85-3.77 (m, 1H), 3.77-3.69 (m, 1H), 2.58-2.46 (m, 1H), 2.42-2.33 (m, 4H), 2.29 (dt, J=4.0, 7.6 Hz, 4H), 1.91-1.78 (m, 1H), 1.77-1.58 (m, 8H), 1.57-1.47 (m, 8H), 1.45-1.37 (m, 4H), 1.36-1.22 (m, 52H), 0.92-0.85 (m, 21H), 0.09 (d, J=4.8 Hz, 6H)

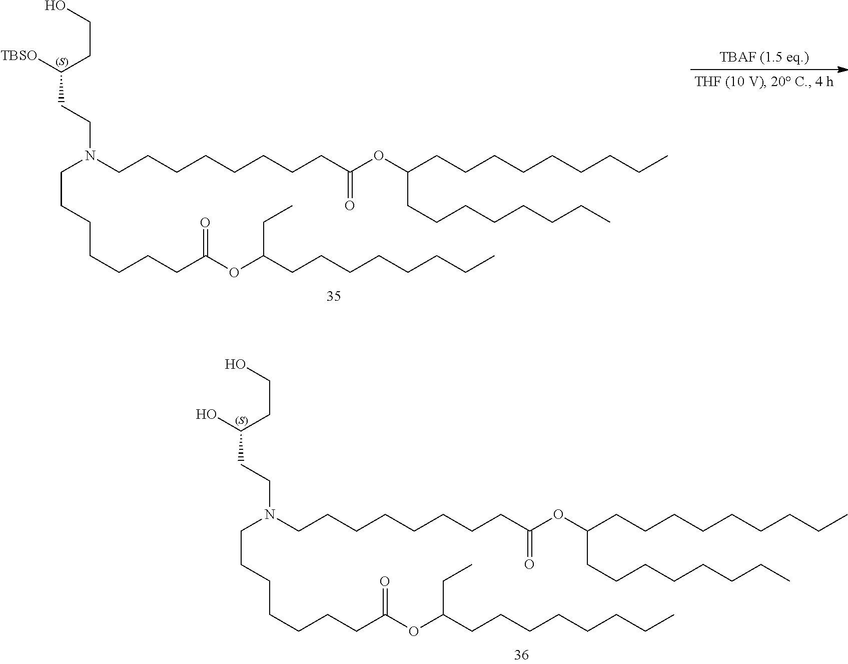

To a solution of Compound 35 (2.04 g, 2.17 mmol, 1 eq.) in THF (20 mL) was added TBAF (1 M, 3.26 mL, 1.5 eq). The mixture was stirred at 20° C. for 4 hours. TLC (Commercial hexanes:Ethyl acetate=1:1, R_{f}=0.1) indicated Compound 35 was consumed, and one major new spot with larger polarity was detected. The reaction mixture was diluted with H₂O (15 mL) and extracted with Ethyl acetate (20 mL×2). The combined organic layers were washed with brine (20 mL), dried over Na₂SO₄, filtered and concentrated under reduced pressure to give a residue. The residue was purified by column chromatography (SiO₂, Commercial hexanes:Ethyl acetate=30:1 to 2:1 to Dichloromethane:Methanol=10:1) to give Compound 36 (1.27 g, 1.52 mmol, 69.76% yield, 98.41% purity) as a light-yellow oil.

LCMS [M+1]⁺=824.7

¹H NMR (400 MHz, CHLOROFORM-d) δ=4.96-4.72 (m, 2H), 4.11-4.00 (m, 1H), 3.84 (t, J=5.6 Hz, 2H), 3.51-3.16 (m, 1H), 2.82-2.66 (m, 1H), 2.64-2.50 (m, 3H), 2.34-2.19 (m, 6H), 1.79-1.58 (m, 8H), 1.55-1.39 (m, 12H), 1.37-1.20 (m, 52H), 1.00-0.75 (m, 12H)

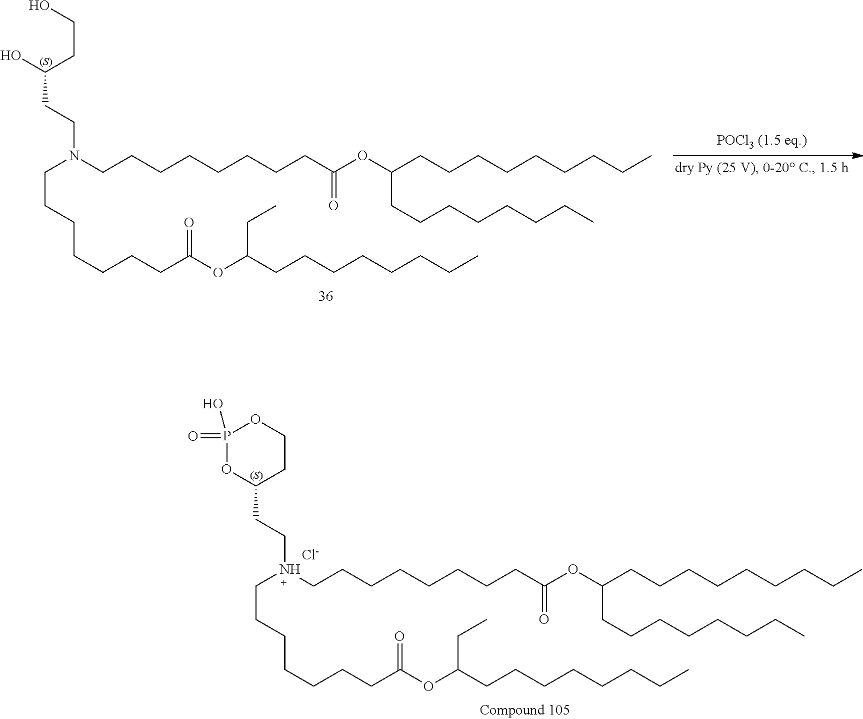

To a cooled solution (0° C.) of Compound 36 (600 mg, 727.85 μmol, 1 eq.) in dry pyridine (15 mL) was added POCl₃ (167.40 mg, 1.09 mmol, 101.77 μL, 1.5 eq.). The mixture was stirred at 20° C. for 1.5 hours. TLC (Dichloromethane:Methanol=10:1, R_{f}=0.18) indicated that Compound 36 was consumed, and one major new spot with larger polarity was detected. The reaction mixture was quenched by the addition H₂O (5 mL) at 0° C. The reaction mixture was concentrated under reduced pressure to give a residue. The residue was co-evaporated with toluene (10 mL) three times at 50° C. to give the crude product. The crude product was purified by prep-HPLC (column: WePure Biotech Phenyl-Hexyl 250×70 7u; mobile phase: [H₂O (0.05% HCl)-ACN:THF=1:1]; gradient: 65%-85% B over 20.0 min) to give the product. The product was purified by column chromatography (SiO₂, Dichloromethane:Methanol=50:1 to 30:1 to 25:1 to 10:1) to give Compound 105 (250 mg, 282.07 μmol, 38.75% yield, Free) as a light-yellow oil.

¹H NMR (400 MHz, CHLOROFORM-d) δ=4.94-4.75 (m, 2H), 4.58-4.32 (m, 2H), 4.27-4.06 (m, 1H), 3.45-2.78 (m, 6H), 2.39-2.22 (m, 4H), 2.21-2.08 (m, 1H), 1.98-1.68 (m, 8H), 1.67-1.43 (m, 14H), 1.41-1.20 (m, 52H), 0.99-0.78 (m, 12H)

To a solution of Compound 105 (230 mg, 259.50 μmol, 1 eq.) in DCM (2.3 mL) was added HCl/dioxane (1 M, 778.51 μL, 3 eq.) at 20° C., and the mixture was stirred at 25° C. for 2 hours. The reaction mixture was concentrated and lyophilized to give Compound 105 (101.2 mg, 110.99 μmol, 100% purity, 0.7 HCl) as light-yellow gum.

HRMS-TOF [M+1]⁺=886.7252

### General Procedure for the Preparation of Compound 106:

¹H NMR (400 MHz, CHLOROFORM-d) δ=11.24 (br s, 1H), 4.95-4.77 (m, 2H), 4.69 (br s, 1H), 4.43 (br t, J=10.0 Hz, 1H), 4.34-4.14 (m, 1H), 3.36 (br s, 2H), 3.19-2.83 (m, 4H), 2.35-2.22 (m, 4H), 2.22-2.01 (m, 2H), 1.93 (br d, J=10.0 Hz, 1H), 1.76 (br d, J=11.2 Hz, 5H), 1.67-1.56 (m, 5H), 1.55-1.45 (m, 7H), 1.43-1.14 (m, 52H), 0.93-0.81 (m, 12H)

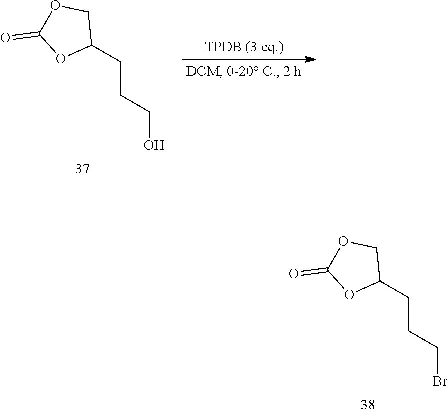 [SMILES: O=C1OCC(CCCO)O1 ; O=C1OCC(CCCBr)O1]

To a solution of Compound 37 (560 mg, 3.83 mmol, 1 eq.) in DCM (20 mL) was added dibromo(triphenyl)-phosphane (4.85 g, 11.5 mmol, 3 eq.) at 0° C. The mixture was stirred at 20° C. for 2 hours. TLC (Commercial hexanes:Ethyl acetate=1:1, R_{f}=0.5) indicated that Compound 37 was consumed completely and many new spots formed. The reaction mixture was concentrated under reduced pressure to give a residue. The residue was purified by column chromatography (SiO₂, Commercial hexanes:Ethyl acetate=10:1 to 3:1) to give Compound 38 (720 mg, 3.44 mmol, 89.9% yield) as a colorless oil.

¹H NMR (400 MHz, CHLOROFORM-d) δ=4.83-4.67 (m, 1H), 4.58 (t, J=8.4 Hz, 1H), 4.11 (dd, J=7.2, 8.4 Hz, 1H), 3.55-3.42 (m, 2H), 2.21-2.07 (m, 1H), 2.06-1.89 (m, 3H)

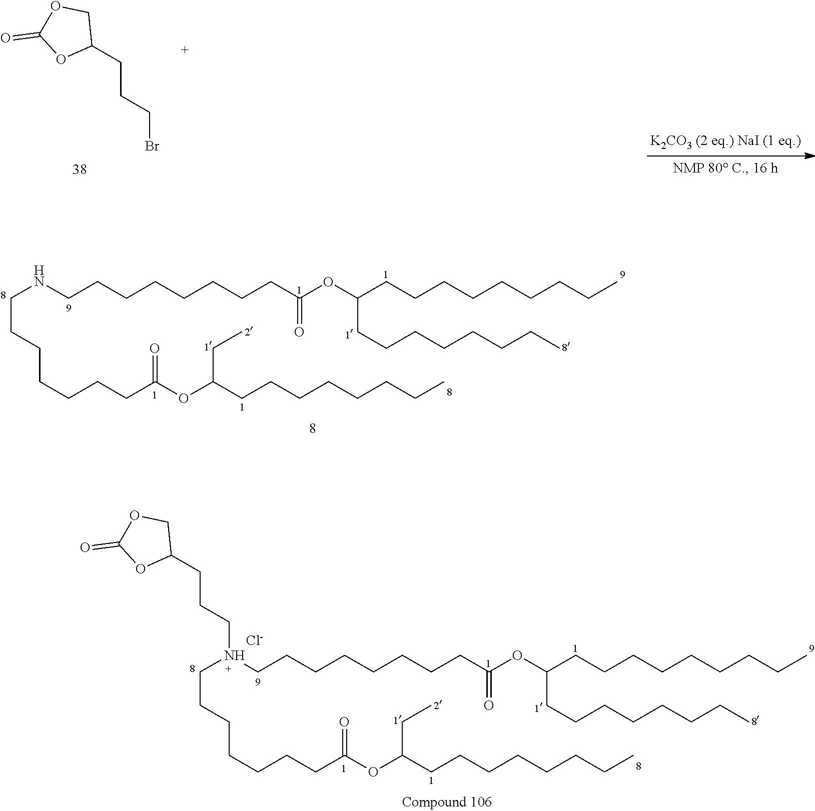 [SMILES: O=C1OCC(CCCBr)O1 ; CCCCCCCCCC(CCCCCCCC)OC(=O)CCCCCCCCNCCCCCCCC(=O)OC(CC)CCCCCCCC ; CCCCCCCCCC(CCCCCCCC)OC(=O)CCCCCCCCN(CCCCCCCC(=O)OC(CC)CCCCCCCC)CCCC1COC(=O)O1 ; [Cl-]]

A mixture of Compound 38 (137 mg, 658 μmol, 0.95 eq.), Compound 8 (500 mg, 692 μmol, 1 eq.), K₂CO₃ (191 mg, 1.38 mmol, 2 eq.), NaI (104 mg, 692 μmol, 1 eq.) in NMP (5 mL) was degassed and purged with N₂ for 3 times, and then the mixture was stirred at 80° C. for 16 hours under N₂ atmosphere. TLC (Commercial hexanes:Ethyl acetate=1:1, R_{f}=0.5) indicated Compound 8 was consumed completely, and many new spots formed. The reaction mixture was diluted with H₂O (20 mL) and extracted with ethyl acetate (20 mL×3). The combined organic layers were washed with brine (20 mL×3), dried over Na₂SO₄, filtered and concentrated under reduced pressure to give a residue. The residue was purified by column chromatography (SiO₂, Dichloromethane:Methanol=96:4 to 95:5) to give crude product. The crude product was purified by prep-HPLC (column: Welchrom CSH C18 100×30×7; mobile phase: [H₂O (0.02% HCl)-ACN:THF=1:1]; gradient: 50%-90% B over 16.0 min) to give Compound 106 (220 mg, 245.57 μmol, 35.47% yield, 98.58% purity, 0.9 HCl) as a colorless oil LCMS [M+1]⁺=850.7

¹H NMR (400 MHz, CHLOROFORM-d) δ 12.16 (br s, 1H), 4.94-4.73 (m, 3H), 4.62 (t, J=8.4 Hz, 1H), 4.14 (dd, J=8.4, 7.2 Hz, 1H), 3.01 (br dd, J=16.8, 10.4 Hz, 6H), 2.29 (q, J=7.2 Hz, 4H), 2.20-1.93 (m, 3H), 1.90-1.70 (m, 5H), 1.68-1.52 (m, 12H), 1.39-1.23 (m, 52H), 0.95-0.81 (m, 12H)

### General Procedure for the Preparation of Compound 108:

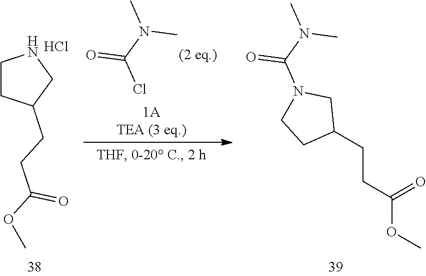

To a solution of Compound 38 (800 mg, 4.13 mmol, 1 eq., HCl) in DMF (8 mL) was added TEA (1.25 g, 12.39 mmol, 1.72 mL, 3 eq.) and Compound 39 (888.43 mg, 8.26 mmol, 758.04 μL, 2 eq.) at 0° C. The mixture was stirred at 20° C. for 2 hours. LCMS showed Compound 38 was consumed completely, and one main peak with desired MS was detected. The reaction mixture was concentrated to give a residue. The residue was diluted with H₂O (30 mL) and extracted with Ethyl acetate (20 mL×3). The combined organic layers were washed with brine (15 mL×2), dried over Na₂SO₄, filtered and concentrated under reduced pressure to give a residue. The residue was purified by column chromatography (Commercial hexanes:Ethyl acetate=100:1 to 80:20) to give Compound 39 (680 mg, 2.54 mmol, 61.40% yield, 85.14% purity) as a colorless oil.

¹H NMR (400 MHz, CHLOROFORM-d) δ=3.69 (s, 3H), 3.53-3.35 (m, 3H), 3.08-2.98 (m, 1H), 2.83 (s, 6H), 2.41-2.32 (m, 2H), 2.19-1.92 (m, 2H), 1.81-1.66 (m, 2H), 1.56-1.39 (m, 1H)

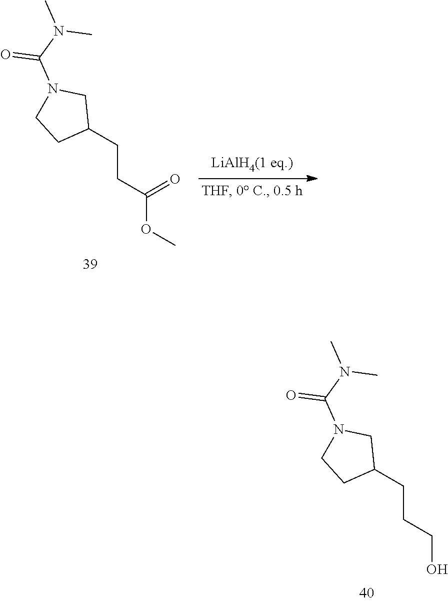 [SMILES: COC(=O)CCC1CCN(C(=O)N(C)C)C1 ; CN(C)C(=O)N1CCC(CCCO)C1]

To a solution of Compound 39 (580 mg, 2.54 mmol, 1 eq.) in THF (6 mL) was added LiAlH₄ (2.5 M, 1.02 mL, 1 eq.) at 0° C. The mixture was stirred at 0° C. for 0.5 hour. LCMS showed Compound 39 was consumed completely and one main peak with desired MS was detected. The reaction mixture was quenched with sodium sulfate decahydrate (1 g) at 0° C., filtered and concentrated under reduced pressure to give a residue. The residue was purified by column chromatography (SiO₂, Commercial hexanes:Ethyl acetate=55:45 to 0:1) to give Compound 40 (417 mg, 2.08 mmol, 81.95% yield, 100% purity) as a light yellow oil.

LCMS [M+1]⁺=201.2

¹H NMR (400 MHz, CHLOROFORM-d) δ=3.67 (t, J=6.4 Hz, 2H), 3.52-3.35 (m, 3H), 3.03 (t, J=9.2 Hz, 1H), 2.83 (s, 6H), 2.19-1.93 (m, 2H), 1.72-1.57 (m, 2H), 1.56-1.51 (m, 1H), 1.50-1.40 (m, 3H)

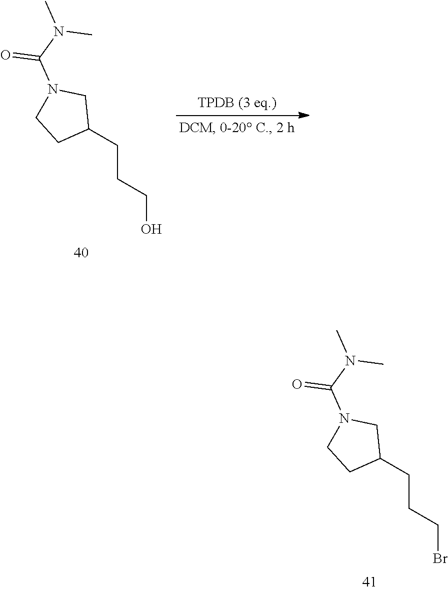

To a solution of Compound 40 (367 mg, 1.83 mmol, 1 eq.) in Dichloromethane (11.7 mL) was added dibromo(triphenyl)-phosphane (2.32 g, 5.50 mmol, 3 eq.) at 0° C. The mixture was stirred at 20° C. for 2 hours. LCMS showed Compound 40 was consumed completely and one main peak with desired MS was detected. The reaction mixture was filtered, and the filtrate was concentrated under reduced pressure evaporated to give a residue. The residue was purified by column chromatography (SiO₂, Commercial hexanes:Ethyl acetate=10:1 to 0:1 and Dichloromethane:Methanol=95:5 to 4:1) to give Compound 41 (340 mg, 529.69 μmol, 17.42% yield, 41% purity) as a yellow oil.

¹H NMR (400 MHz, CHLOROFORM-d) δ=3.93-3.61 (m, 3H), 3.43 (t, J=6.4 Hz, 2H), 3.28 (br t, J=9.6 Hz, 1H), 3.07 (s, 6H), 2.40-2.09 (m, 2H), 2.01-1.84 (m, 2H), 1.75-1.54 (m, 3H)

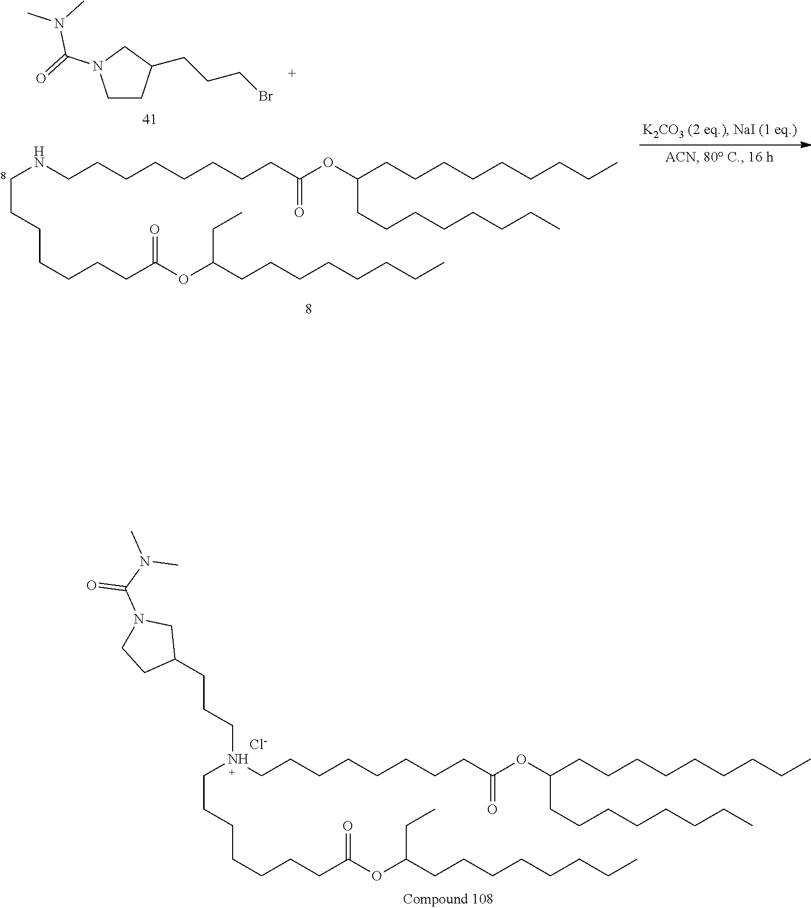 [SMILES: CN(C)C(=O)N1CCC(CCCBr)C1 ; CCCCCCCCCC(CCCCCCCC)OC(=O)CCCCCCCCNCCCCCCCC(=O)OC(CC)CCCCCCCC ; CCCCCCCCCC(CCCCCCCC)OC(=O)CCCCCCCCN(CCCCCCCC(=O)OC(CC)CCCCCCCC)CCCC1CCN(C(=O)N(C)C)C1 ; [Cl-]]

To a solution of Compound 41 (110 mg, 417.97 μmol, 1 eq.), Compound 8 (301.87 mg, 417.97 μmol, 1 eq.) in ACN (1.1 mL) was added K₂CO₃ (115.53 mg, 835.95 μmol, 2 eq.) and NaI (62.65 mg, 417.97 μmol, 1 eq.) at 20° C. The mixture was stirred at 80° C. for 16 hours. LCMS showed that Compound 41 was consumed completely and one main peak with the desired MS was detected. After filtration via Celite pad, the filtrate was concentrated to give a residue. The residue was purified by column chromatography (SiO₂, Commercial hexanes:Ethyl acetate=99:1 to 0:1) to give a mixture of Compound 108 and Compound 8 (328 mg) as a colorless oil.

To the mixture (320 mg) in dichloromethane (3 mL) was added TEA (31.68 mg, 313.11 μmol, 43.58 μL, 1.2 eq.) and tert-butoxycarbonyl tert-butyl carbonate (56.95 mg, 260.92 μmol, 59.94 μL, 1 eq.). The mixture was stirred at 20° C. for 1 hour. LCMS showed Compound 8 was consumed completely and one main peak with desired MS was detected. The reaction mixture was diluted with H₂O (30 mL) and extracted with Dichloromethane (20 mL×3). The combined organic layers were washed with brine (15 mL×2), dried over Na₂SO₄, filtered and concentrated under reduced pressure to give a residue. The residue was purified by column chromatography (Commercial hexanes:Ethyl acetate=80:20 to 0:1) and re-purified by prep-HPLC (column: Welchrom CSH C18 100×30×7; mobile phase: [H₂O (0.02% HCl)-ACN:THF=1:1]; gradient: 50%-90% B over 14.0 min) to give Compound 108 (100.76 mg, 107.26 μmol, 41.11% yield, 99.78% purity, 0.9 HCl) as a colorless oil.

HRMS-TOF [M+1]⁺=904.8

¹H NMR (400 MHz, CHLOROFORM-d) δ=12.33-11.91 (m, 1H), 4.95-4.72 (m, 2H), 3.60-3.32 (m, 3H), 3.13-2.91 (m, 6H), 2.84 (br s, 6H), 2.29 (br d, J=5.2 Hz, 4H), 2.18-2.08 (m, 1H), 2.03 (br d, J=2.0 Hz, 1H), 1.95-1.74 (m, 6H), 1.72-1.55 (m, 14H), 1.45-1.14 (m, 54H), 0.88 (br s, 12H)

### General Procedure for the Preparation of Compound 109:

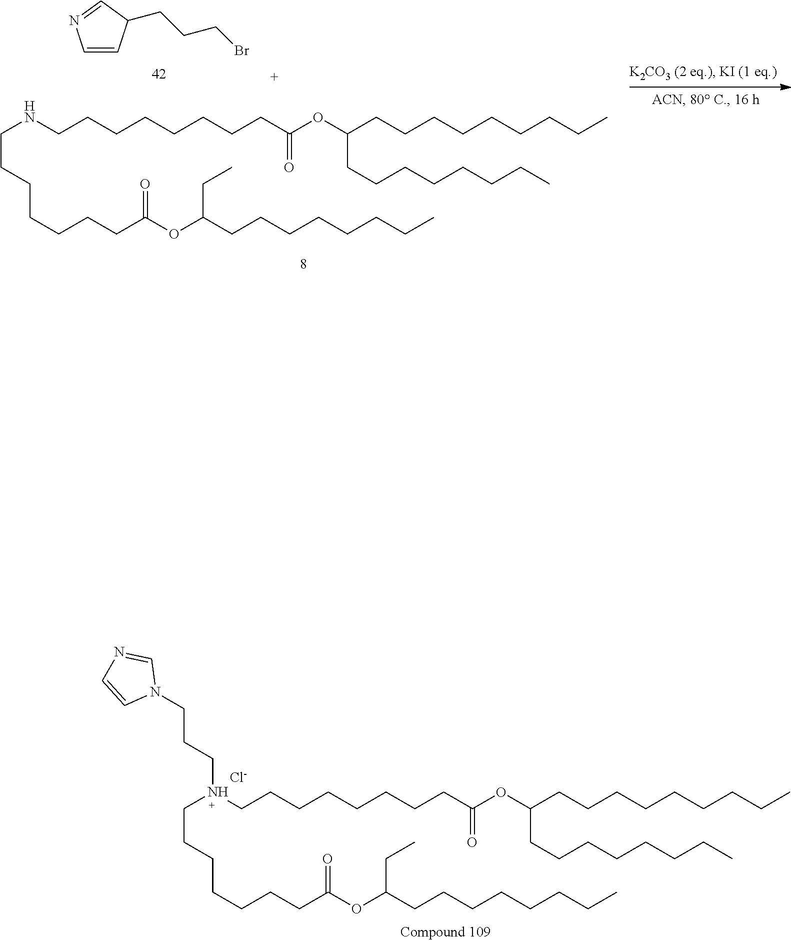 [SMILES: BrCCCC1C=CN=C1 ; CCCCCCCCCC(CCCCCCCC)OC(=O)CCCCCCCCNCCCCCCCC(=O)OC(CC)CCCCCCCC ; CCCCCCCCCC(CCCCCCCC)OC(=O)CCCCCCCCN(CCCCCCCC(=O)OC(CC)CCCCCCCC)CCCn1ccnc1 ; [Cl-]]

To a solution of Compound 42 (117.80 mg, 623.08 μmol, 0.9 eq.) in NMP (5 mL) was added Compound 8 (500 mg, 692.31 μmol, 1 eq.), K₂CO₃ (191.36 mg, 1.38 mmol, 2 eq.) and NaI (103.77 mg, 692.31 μmol, 1 eq.). The mixture was stirred at 80° C. for 16 hours. TLC (Dichloromethane:Methanol=20:1, R_{f}=0.4) indicated Compound 42 was consumed completely and many new spots formed. The mixture was filtered through a Celite pad, and the filtrate was diluted with ethyl acetate (50 mL). The combined organic layers were washed with brine (30 mL×5), dried over Na₂SO₄, filtered and concentrated under reduced pressure to give a residue. The residue was purified by column chromatography (SiO₂, DCM:MeOH=96:4 to 94:6) to give crude product. The crude product was re-purified by prep-HPLC (column: X-Select CSH Phenyl-Hexyl 100×30 5u; mobile phase: [H₂O (0.02% HCl)-ACN:THF=1:1]; gradient: 40%-80% B over 16.0 min) to give Compound 109 (220 mg, 243.32 μmol, 35.15% yield, 99.5% purity, 1.9 HCl) as a white solid.

HRMS-TOF [M+1]⁺=830.7

¹H NMR (400 MHz, DMSO-d₆) δ=15.01-14.03 (m, 1H), 10.67-10.42 (m, 1H), 9.11 (s, 1H), 7.92-7.54 (m, 2H), 4.87-4.64 (m, 2H), 4.29 (br t, J=7.2 Hz, 2H), 3.00 (br d, J=1.2 Hz, 6H), 2.27 (q, J=7.2 Hz, 6H), 1.61 (br s, 4H), 1.56-1.40 (m, 12H), 1.31-1.16 (m, 52H), 0.93-0.76 (m, 12H)

### General Procedure for the Preparation of Compound 110:

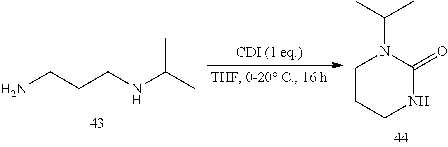

To a solution of Compound 43 (5.15 g, 44.32 mmol, 6.20 mL, 1 eq.) in THF (51.5 mL) was added CDI (7.19 g, 44.32 mmol, 1 eq.) at 0° C. The mixture was stirred at 20° C. for 16 hours. TLC (Dichloromethane:Methanol=10:1, R_{f}=0.62) indicated that Compound 43 was consumed completely and one major new spot formed. HCl (1 M) was added to adjust the pH below 3 and the solution was extracted with DCM (20 mL×5). The combined organic layers were dried with anhydrous Na₂SO₄, filtered and concentrated under vacuum to give Compound 44 (5.05 g, 35.51 mmol, 80.13% yield) as a white solid.

¹H NMR (400 MHz, CHLOROFORM-d) δ=5.11-4.80 (m, 1H), 4.71 (td, J=6.8, 13.6 Hz, 1H), 3.26 (t, J=5.6 Hz, 2H), 3.14 (t, J=5.6 Hz, 2H), 1.98-1.80 (m, 2H), 1.09 (d, J=6.8 Hz, 6H)

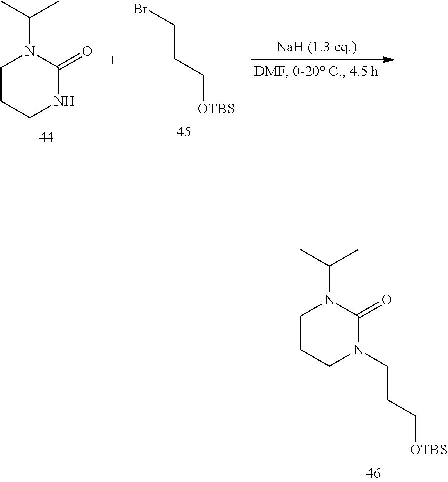

NaH (1.54 g, 38.50 mmol, 60% purity, 1.3 eq.) was added to a solution of Compound 44 (4.21 g, 29.61 mmol, 1 eq.) in DMF (42 mL) at 0° C. The reaction temperature was allowed to rise to 20° C. and the reaction mixture was stirred for 30 min. Then, Compound 45 (9 g, 35.54 mmol, 1.2 eq.) was added at 20° C. The mixture was stirred at 20° C. for 4 hours. TLC (Dichloromethane:Methanol=15:1, R_{f}=0.67) indicated Compound 44 was consumed completely and one major new spot formed. The reaction mixture was quenched by addition saturated NH₄Cl (50 mL) at 20° C., and then extracted with ethyl acetate (80 mL×3). The combined organic layers were concentrated under reduced pressure to give a residue. The residue was purified by flash silica gel chromatography (ISCO®; 80 g SepaFlash® Silica Flash Column, Eluent of 5-50% Ethylacetate/Commercial hexanes gradient @150 mL/min) to give Compound 46 (5.04 g, 9.54 mmol, 32.20% yield, 59.52% purity) as a colorless oil.

LCMS [M+1]⁺=315.3

¹H NMR (400 MHz, CHLOROFORM-d) δ=4.75 (td, J=6.8, 13.6 Hz, 1H), 3.66 (t, J=6.4 Hz, 2H), 3.38 (t, J=7.2 Hz, 2H), 3.25 (t, J=6.0 Hz, 2H), 3.12 (t, J=6.0 Hz, 2H), 1.90 (quin, J=6.0 Hz, 2H), 1.77 (quin, J=6.8 Hz, 2H), 1.08 (d, J=6.8 Hz, 6H), 0.90 (s, 9H), 0.05 (s, 6H)

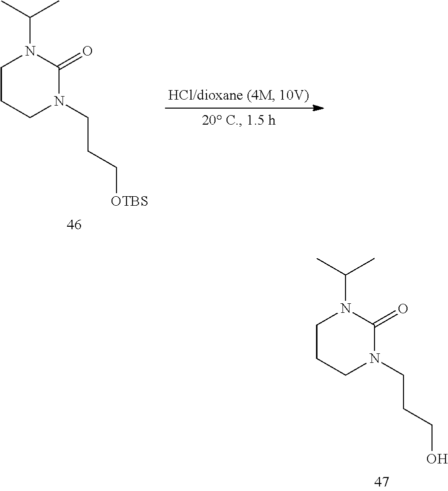 [SMILES: CC(C)N1CCCN(CCCO[Si](C)(C)C(C)(C)C)C1=O ; CC(C)N1CCCN(CCCO)C1=O]

A solution of Compound 46 (5.04 g, 16.02 mmol, 1 eq.) in HCl/dioxane (4 M, 50.40 mL, 12.58 eq.) at 20° C. was stirred at 20° C. for 1.5 hours. TLC (Dichloromethane:Methanol=10:1, R_{f}=0.53) indicated Compound 46 remained and one major new spot formed. The reaction mixture was diluted with DCM (200 mL) and basified with Sat. NaHCO₃ (300 mL) at 0° C. adjusted to pH=7. The separated organic layer was dried over Na₂SO₄ and evaporated to give a residue. The residue was purified by column chromatography (SiO₂, Dichloromethane:Methanol=100:0 to 10:1) to give Compound 47 (696 mg, 3.42 mmol, 21.33% yield, 98.37% purity) as a light yellow oil.

LCMS [M+1]⁺=201.4

¹H NMR (400 MHz, CHLOROFORM-d) δ=4.72 (td, J=6.8, 13.6 Hz, 1H), 3.59-3.44 (m, 4H), 3.19 (td, J=6.0, 18.8 Hz, 4H), 1.93 (quin, J=6.0 Hz, 2H), 1.66 (td, J=5.6, 11.2 Hz, 2H), 1.09 (d, J=6.8 Hz, 6H)

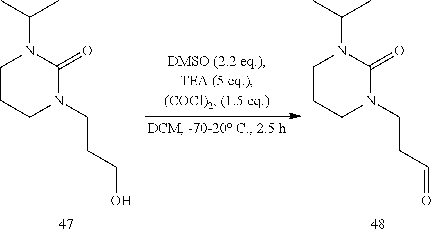

To a solution of (COCl)₂ (285.19 mg, 2.25 mmol, 196.68 μL, 1.5 eq.) in DCM (5 mL) was added DMSO (257.48 mg, 3.30 mmol, 257.48 μL, 2.2 eq.) dropwise at −70° C. The mixture was stirred at −70° C. for 0.5 hour. Compound 47 (300 mg, 1.50 mmol, 1 eq.) in DCM (3 mL) was added dropwise at −70° C., and the mixture was stirred at −70° C. for 1.5 hour. TEA (757.87 mg, 7.49 mmol, 1.04 mL, 5 eq.) was added at −70° C., and the mixture was stirred at 20° C. for 0.5 hour. LCMS indicated Compound 47 was consumed completely, and one main peak with desired MS was detected. The reaction mixture was diluted with H₂O (50 mL) and extracted with DCM (50 mL×2). The combined organic layers were dried over Na₂SO₄, filtered and concentrated under reduced pressure to give Compound 48 (252 mg, 1.23 mmol, 81.82% yield, 96.43% purity) as a light yellow oil.

LCMS [M+1]⁺=199.3

¹H NMR (400 MHz, CHLOROFORM-d) δ=9.83 (t, J=2.0 Hz, 1H), 4.70 (td, J=6.8, 13.6 Hz, 1H), 3.64 (t, J=6.4 Hz, 2H), 3.28 (t, J=6.0 Hz, 2H), 3.15-3.03 (m, 2H), 2.73 (dt, J=1.6, 6.4 Hz, 2H), 1.90 (quin, J=6.0 Hz, 2H), 1.07 (d, J=6.8 Hz, 6H)

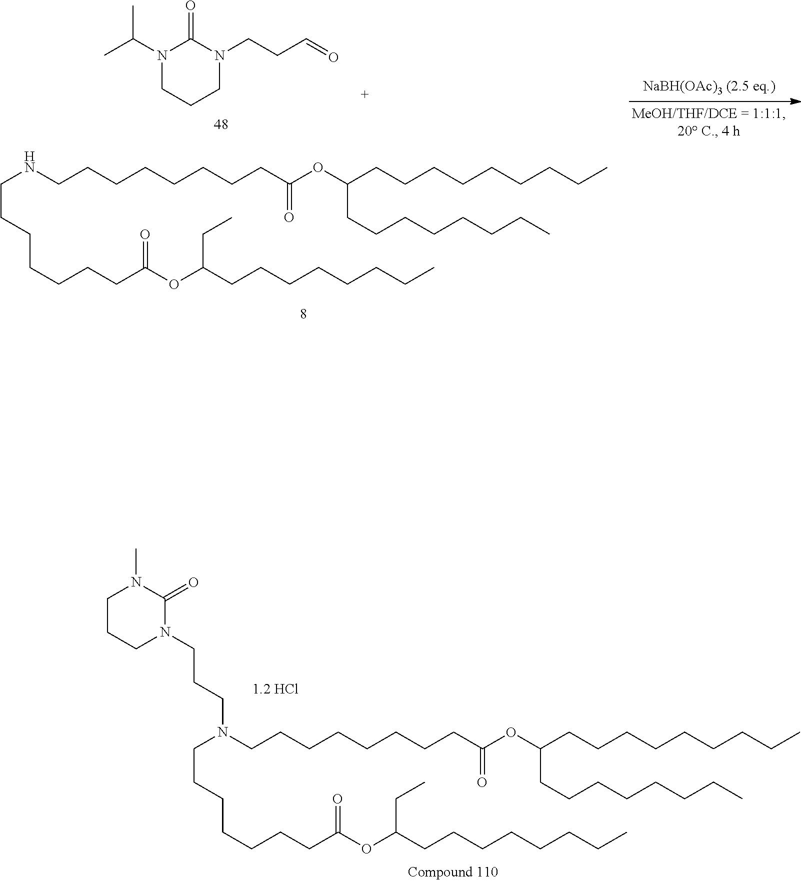 [SMILES: CC(C)N1CCCN(CCC=O)C1=O ; CCCCCCCCCC(CCCCCCCC)OC(=O)CCCCCCCCNCCCCCCCC(=O)OC(CC)CCCCCCCC ; CCCCCCCCCC(CCCCCCCC)OC(=O)CCCCCCCCN(CCCCCCCC(=O)OC(CC)CCCCCCCC)CCCN1CCCN(C)C1=O]

A solution of Compound 8 (230.60 mg, 1.16 mmol, 1.2 eq.), Compound 48 (700 mg, 969.24 μmol, 1 eq.) and 4A MS (700 mg) in MeOH (2 mL) and DCE (2 mL) was stirred for 2 hours. Then, NaBH(OAc)₃ (513.55 mg, 2.42 mmol, 2.5 eq.) in THF (2 mL) was added, and the mixture was stirred at 20° C. for 2 hours under an N₂ atmosphere. LCMS indicated Compound 8 was consumed completely and one main peak with desired MS was detected. The reaction mixture was diluted with brine (20 mL) and extracted with DCM (50 mL×2). The combined organic layers were dried over Na₂SO₄, filtered and concentrated under reduced pressure to give a residue. The residue was purified by flash silica gel chromatography (ISCO®; 12 g SepaFlash® Silica Flash Column, Eluent of 5-50% Methanol/Dichloromethane gradient @150 mL/min) to give Compound 110 (401 mg, 427.21 μmol, 44.08% yield, 96.36% purity) as a colorless oil.

LCMS [M+1]⁺=904.8

¹H NMR (400 MHz, CHLOROFORM-d) δ=4.95-4.76 (m, 2H), 4.69 (td, J=6.8, 13.6 Hz, 1H), 3.43 (t, J=6.4 Hz, 2H), 3.33 (t, J=6.0 Hz, 2H), 3.17 (t, J=6.0 Hz, 2H), 3.07-2.92 (m, 6H), 2.30 (dt, J=4.0, 7.2 Hz, 4H), 2.21-2.12 (m, 2H), 1.97 (quin, J=5.6 Hz, 2H), 1.79 (br s, 4H), 1.67-1.58 (m, 6H), 1.55-1.48 (m, 6H), 1.39-1.22 (m, 52H), 1.10 (d, J=6.8 Hz, 6H), 0.90 (br t, J=6.4 Hz, 12H)

Compound 110 (401 mg, 427.21 μmol, 44.08% yield, 96.36% purity) was re-purified by prep-HPLC (column: X-Select CSH Phenyl-Hexyl 100×30 5u; mobile phase: [H₂O (0.02% HCl)-ACN:THF=1:1]; gradient: 50%-90% B over 14.0 min) to give Compound 110 (307 mg, 321.75 μmol, 72.57% yield, 99.38% purity, 1.2 HCl) as a colorless oil.

LCMS [M+1]⁺=904.8

¹H NMR (400 MHz, CHLOROFORM-d) δ=12.02-11.78 (m, 1H), 4.93-4.77 (m, 2H), 4.76-4.58 (m, 1H), 3.52-3.42 (m, 2H), 3.36 (br s, 2H), 3.18 (br d, J=5.6 Hz, 2H), 3.10-2.88 (m, 6H), 2.29 (dt, J=4.8, 7.2 Hz, 4H), 2.20 (br d, J=6.0 Hz, 2H), 2.02-1.92 (m, 2H), 1.77 (br d, J=4.0 Hz, 4H), 1.66-1.45 (m, 12H), 1.43-1.22 (m, 52H), 1.11 (d, J=6.8 Hz, 6H), 0.98-0.78 (m, 12H)

### General Procedure for the Preparation of Compound 111:

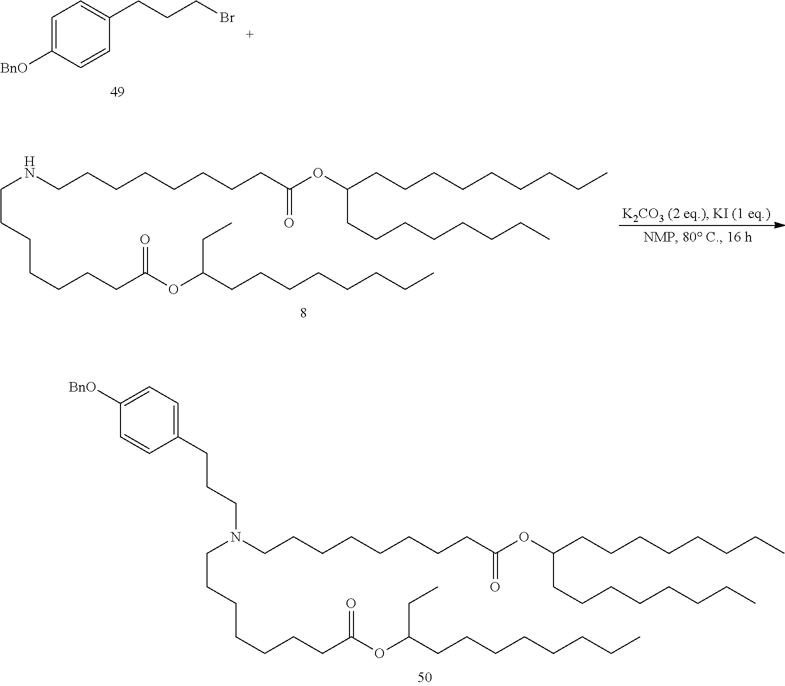 [SMILES: BrCCCc1ccc(OCc2ccccc2)cc1 ; CCCCCCCCCC(CCCCCCCC)OC(=O)CCCCCCCCNCCCCCCCC(=O)OC(CC)CCCCCCCC ; CCCCCCCCC(CC)OC(=O)CCCCCCCN(CCCCCCCCC(=O)OC(CCCCCCCC)CCCCCCCC)CCCc1ccc(OCc2ccccc2)cc1]

To a solution of Compound 49 (190.17 mg, 623.08 μmol, 0.9 eq.) in NMP (5 mL) was added K₂CO₃ (191.36 mg, 1.38 mmol, 2 eq.), Compound 8 (500 mg, 692.31 μmol, 1 eq.) and NaI (103.77 mg, 692.31 μmol, 1 eq.) at 20° C. The mixture was stirred at 80° C. for 16 hours. LCMS showed Compound 8 was consumed completely, and one main peak with desired MS was detected. The reaction mixture was diluted with H₂O (15 mL) and extracted with ethyl acetate (10 mL×3). The combined organic layers were washed with brine (10 mL×2), dried over Na₂SO₄, filtered and concentrated under reduced pressure to give a residue. The residue was purified by column chromatography (SiO₂, Commercial hexanes:Ethyl acetate=30:1 to 10:1) to give Compound 50 (466 mg, 492.33 μmol) as a light yellow oil.

LCMS [M+1]⁺=946.8

¹H NMR (400 MHz, CHLOROFORM-d) δ=7.48-7.42 (m, 2H), 7.42-7.36 (m, 2H), 7.36-7.29 (m, 1H), 7.11 (d, J=8.4 Hz, 2H), 6.91 (d, J=8.4 Hz, 2H), 5.05 (s, 2H), 4.94-4.74 (m, 2H), 2.61-2.50 (m, 2H), 2.49-2.33 (m, 5H), 2.29 (dt, J=4.4, 7.6 Hz, 4H), 1.81-1.67 (m, 2H), 1.63 (br s, 4H), 1.56-1.46 (m, 8H), 1.40 (br s, 4H), 1.35-1.21 (m, 52H), 0.92-0.85 (m, 12H)

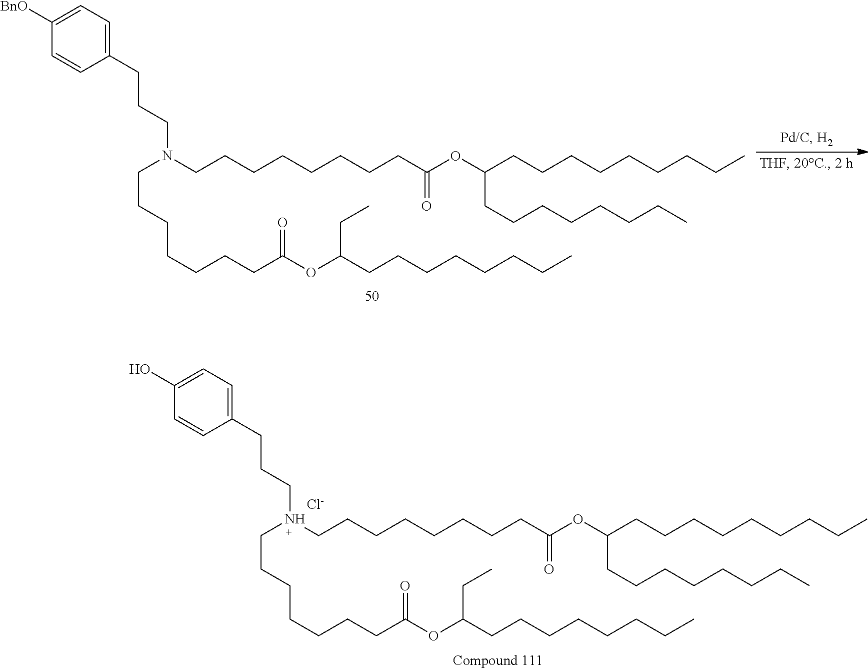 [SMILES: CCCCCCCCCC(CCCCCCCC)OC(=O)CCCCCCCCN(CCCCCCCC(=O)OC(CC)CCCCCCCC)CCCc1ccc(OCc2ccccc2)cc1 ; CCCCCCCCCC(CCCCCCCC)OC(=O)CCCCCCCCN(CCCCCCCC(=O)OC(CC)CCCCCCCC)CCCc1ccc(O)cc1 ; [Cl-]]

To a solution of Compound 56 (410 mg, 433.17 μmol, 1 eq.) in THF (4 mL) was added Pd/C (92.20 mg, 86.63 μmol, 10% purity, 0.2 eq.) at 20° C. and the solution was purged with H₂ for 3 times. The mixture was stirred at 20° C. for 16 hours under H₂ atmosphere. LCMS showed Compound 56 was consumed completely, and one main peak with desired MS was detected. The reaction mixture was filtered and the filtrate was concentrated to give a residue. The residue was purified by column chromatography (Commercial hexanes:Ethyl acetate=50:1 to 30:1) to give Compound 111 (Free) (266 mg, 310.61 μmol) as a colorless oil.

LCMS [M+1]⁺=856.8

¹H NMR (400 MHz, CHLOROFORM-d) δ=7.11-6.97 (m, 2H), 6.76 (br d, J=8.0 Hz, 2H), 4.96-4.73 (m, 2H), 3.02-2.16 (m, 12H), 1.82-1.67 (m, 2H), 1.57 (br s, 6H), 1.52 (br s, 6H), 1.42-1.34 (m, 4H), 1.27 (br s, 52H), 0.89 (t, J=6.8 Hz, 12H)

### General Procedure for the Preparation of Compound 112:

A mixture of TMSCl (168.72 mg, 1.55 mmol, 197.11 μL, 5 eq.) and TFE (155.36 mg, 1.55 mmol, 111.69 μL, 5 eq.) was stirred at 20° C. for 1 hour. Then, Compound 111 (Free) (266 mg, 310.61 μmol, 1 eq.) in DCM (3 mL) was added. The mixture was stirred at 20° C. for 2 hours. The reaction mixture was concentrated under reduced pressure to give crude product. The crude product was purified by prep-HPLC (column: Welchrom CSH C18 100×30×7; mobile phase: [H₂O (0.02% HCl)-ACN:THF=1:1]; gradient: 50%-90% B over 12.0 min) to give Compound 111 (180 mg, 201.60 μmol, 64.91% yield, 1.1 HCl) as a colorless oil.

LCMS [M+1]⁺=856.8

¹H NMR (400 MHz, CHLOROFORM-d) δ=12.09-11.89 (m, 1H), 7.02 (br d, J=8.4 Hz, 2H), 6.81 (br d, J=8.4 Hz, 2H), 6.61-6.37 (m, 1H), 5.04-4.76 (m, 2H), 3.04-2.75 (m, 6H), 2.64 (br t, J=5.6 Hz, 2H), 2.31 (dt, J=2.4, 7.2 Hz, 4H), 2.15-1.96 (m, 2H), 1.81-1.58 (m, 8H), 1.55-1.42 (m, 8H), 1.27 (br s, 52H), 0.89 (br t, J=6.8 Hz, 12H)

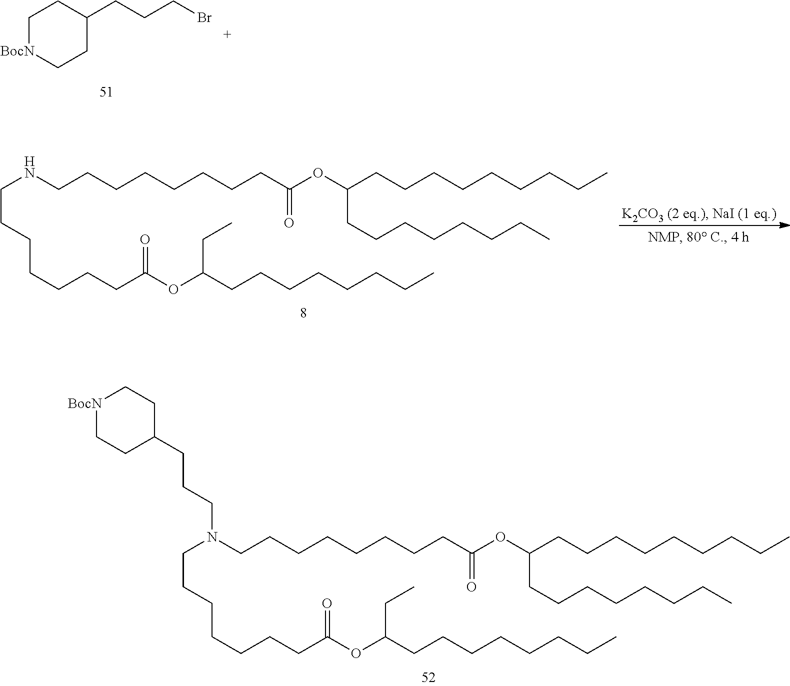 [SMILES: CC(C)(C)OC(=O)N1CCC(CCCBr)CC1 ; CCCCCCCCCC(CCCCCCCC)OC(=O)CCCCCCCCNCCCCCCCC(=O)OC(CC)CCCCCCCC ; CCCCCCCCCC(CCCCCCCC)OC(=O)CCCCCCCCN(CCCCCCCC(=O)OC(CC)CCCCCCCC)CCCC1CCN(C(=O)OC(C)(C)C)CC1]

To a solution of Compound 8 (500 mg, 692.31 μmol, 1 eq.) in NMP (5 mL) was added Compound 51 (190.81 mg, 623.08 μmol, 0.9 eq.), K₂CO₃ (191.36 mg, 1.38 mmol, 2 eq.) and NaI (103.77 mg, 692.31 μmol, 1 eq.). The mixture was stirred at 80° C. for 4 hours. TLC (Dichloromethane:Methanol=10:1, R_{f}=0.58) indicated that Compound 8 remained, and new spots formed. The reaction mixture was diluted with ethyl acetate (40 mL) and was washed with brine (20 mL×2), the organic layer was dried over Na₂SO₄, filtered and concentrated under reduced pressure to give a residue. The residue was purified by column chromatography (SiO₂, Commercial hexanes:Ethyl acetate=20:1 to 4:1) to give crude product (450 mg). The crude product (450 mg) was re-purified by column chromatography (SiO₂, Commercial hexanes:Ethyl acetate=15:1 to 4:1) to give Compound 52 (400 mg, 397.66 μmol, 57.44% yield, 94.20% purity) as a white solid.

¹H NMR (400 MHz, CHLOROFORM-d) δ=4.93-4.75 (m, 2H), 4.19-3.97 (m, 2H), 2.76-2.58 (m, 2H), 2.44-2.33 (m, 5H), 2.29 (dt, J=4.4, 7.6 Hz, 4H), 1.69-1.58 (m, 6H), 1.54-1.49 (m, 6H), 1.46 (s, 9H), 1.42 (br d, J=4.8 Hz, 4H), 1.36-1.21 (m, 52H), 1.14-1.03 (m, 2H), 0.95-0.81 (m, 12H)

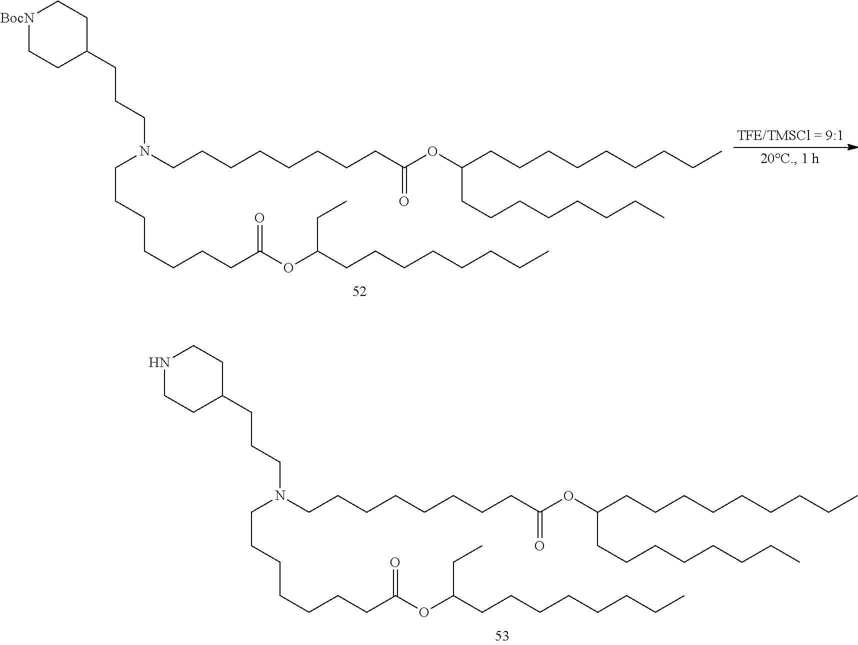 [SMILES: CCCCCCCCCC(CCCCCCCC)OC(=O)CCCCCCCCN(CCCCCCCC(=O)OC(CC)CCCCCCCC)CCCC1CCN(C(=O)OC(C)(C)C)CC1 ; CCCCCCCCCC(CCCCCCCC)OC(=O)CCCCCCCCN(CCCCCCCC(=O)OC(CC)CCCCCCCC)CCCC1CCNCC1]

A solution of Compound 52 (400 mg, 422.15 μmol, 1 eq.) in TFE (3.6 mL) and TMSCl (0.4 mL) was stirred at 20° C. for 1 hour. TLC (Dichloromethane:Methanol=10:1, R_{f}=0.07) indicated Compound 52 was consumed completely, and new spots formed. The reaction mixture was concentrated under reduced pressure to give the crude product (HCl salt). The crude product was dissolved in DCM (10 mL). The solution was cooled to 0° C., Sat. NaHCO₃ (˜2 mL) was added in portions to the mixture adjust to pH=7-8 at 0° C. Then, the mixture was poured into a separatory funnel and separated. The aqueous layer was extracted with dichloromethane (10 mL×2). The combined organic layers were dried over Na₂SO₄, filtered, and the filtrate was concentrated to give Compound 53 (360 mg, 399.03 μmol, 94.52% yield, 93.93% purity) as a light yellow oil.

¹H NMR (400 MHz, CHLOROFORM-d) δ=4.93-4.76 (m, 2H), 3.95 (q, J=8.8 Hz, 1H), 3.16-3.02 (m, 2H), 2.60 (dt, J=2.4, 12.4 Hz, 2H), 2.39 (br s, 6H), 2.29 (dt, J=4.4, 7.6 Hz, 4H), 1.73-1.60 (m, 6H), 1.60-1.48 (m, 8H), 1.42 (br t, J=4.8 Hz, 6H), 1.36-1.20 (m, 52H), 1.15-1.05 (m, 2H), 0.95-0.81 (m, 12H)

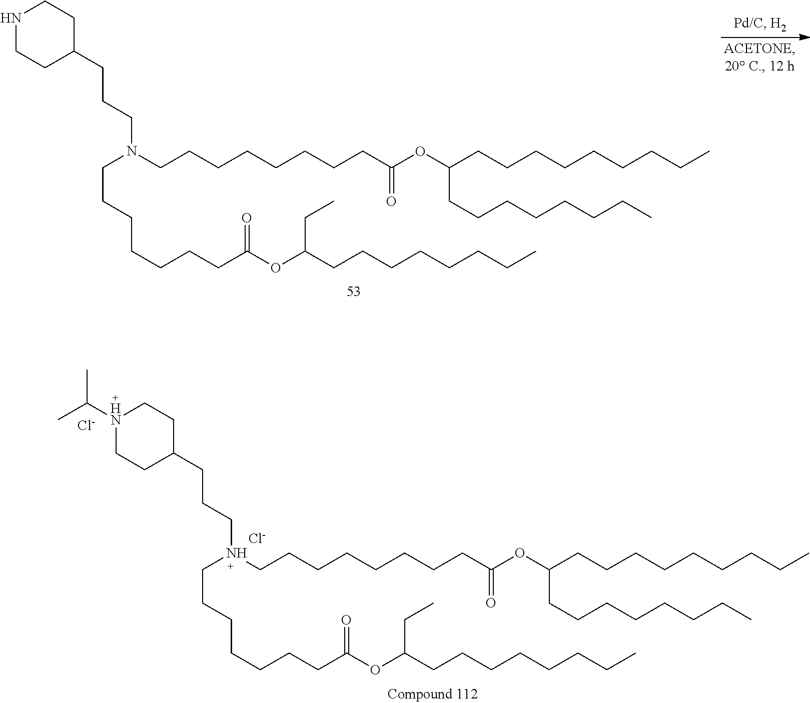

To a solution of Compound 53 (360 mg, 424.82 μmol, 1 eq.) in acetone (3.6 mL) was added Pd/C (117.54 mg, 110.45 μmol, 10% purity, 0.26 eq.) under an N₂ atmosphere. The suspension was degassed and purged with H₂ three times. The mixture was stirred under H₂ (15 Psi) at 20° C. for 12 hours. TLC (Dichloromethane:Methanol=10:1, R_{f}=0.55) indicated Compound 53 was consumed completely and new spots formed. The mixture was filtered through a Celite pad, and the filtrate was concentrated to give crude product. The crude product was purified by column chromatography (SiO₂, Dichloromethane:Methanol=50:1 to 0:1) to give crude product 250 mg, and the crude product was re-purified by prep-HPLC (column: X-Select CSH Phenyl-Hexyl 100*30 5u; mobile phase: [H₂O (0.02% HCl)-ACN]; gradient: 50%-85% B over 12.0 min) to give Compound 112 (170 mg, 176.64 μmol, 41.58% yield, 100% purity, 2.0 HCl) as a white solid.

HRMS-TOF [M+1]⁺=889.8710

¹H NMR (400 MHz, CHLOROFORM-d) δ=12.11-11.95 (m, 1H), 11.90-11.74 (m, 1H), 4.94-4.75 (m, 2H), 3.50-3.30 (m, 3H), 3.10-2.83 (m, 6H), 2.79-2.61 (m, 2H), 2.36-2.10 (m, 6H), 1.94 (br s, 4H), 1.78 (br d, J=1.6 Hz, 7H), 1.68-1.57 (m, 6H), 1.56-1.50 (m, 6H), 1.44 (br d, J=6.4 Hz, 6H), 1.39-1.22 (m, 52H), 0.88 (br t, J=6.4 Hz, 12H)

### General Procedure for the Preparation of Compound 113:

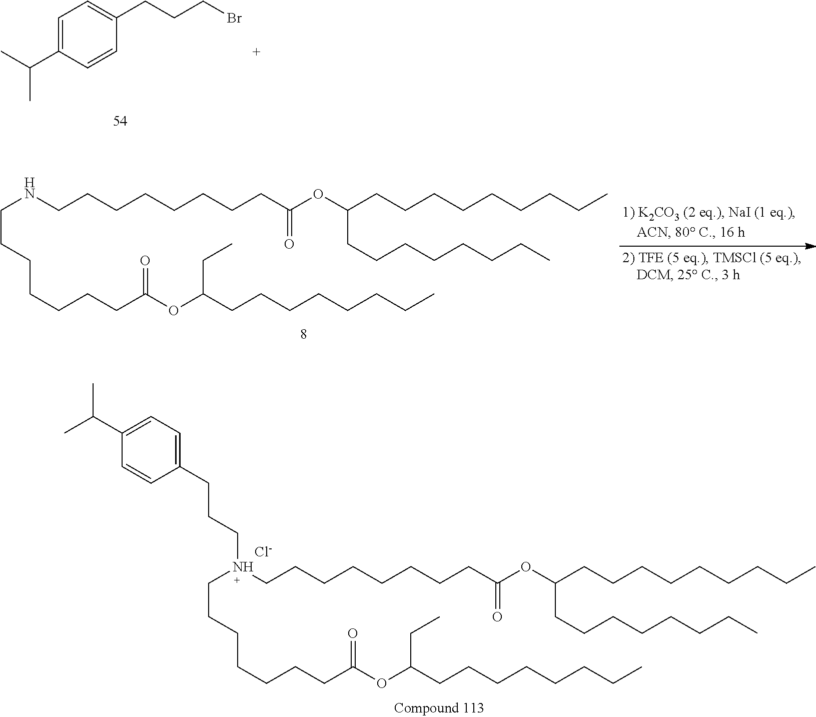

To a solution of Compound 8 (500 mg, 692.31 μmol, 1 eq.) in ACN (5 mL) was added Compound 54 (158.61 mg, 657.70 μmol, 0.95 eq.), K₂CO₃ (191.37 mg, 1.38 mmol, 2 eq.) and NaI (103.77 mg, 692.31 μmol, 1 eq.) at 20° C., and the mixture was heated to 80° C. for 16 hours. LCMS showed Compound 8 remained and one peak with desired MS was detected. The reaction mixture was filtered and the filtrate was concentrated to give a residue. The residue was purified by column chromatography (SiO₂, Commercial hexanes:Ethyl acetate=100:1 to 5:1) to give Compound 113 (Free) (420 mg, 467.85 μmol, 67.58% yield, 98.3% purity) as colorless oil.

LCMS [M+1]⁺=882.8

¹H NMR (400 MHz, CHLOROFORM-d) δ=7.20-7.09 (m, 4H), 4.95-4.77 (m, 2H), 2.88 (td, J=6.8, 14.0 Hz, 1H), 2.62-2.54 (m, 2H), 2.45 (br t, J=7.2 Hz, 2H), 2.42-2.33 (m, 4H), 2.29 (dt, J=4.0, 7.6 Hz, 4H), 1.75 (br t, J=7.2 Hz, 2H), 1.62 (br dd, J=2.0, 7.2 Hz, 4H), 1.54-1.47 (m, 6H), 1.46-1.37 (m, 4H), 1.36-1.19 (m, 60H), 0.97-0.81 (m, 12H)

A solution of TMSCl (123.11 mg, 1.13 mmol, 143.82 μL, 5 eq.) in TFE (113.36 mg, 1.13 mmol, 81.50 μL, 5 eq.) was stirred at 25° C. for 1 hour, and then Compound 113 (Free) (200 mg, 226.64 μmol, 1 eq.) in Dichloromethane (2 mL) was added dropwise at 25° C. The resulting mixture was stirred at 25° C. for 2 hours. The reaction mixture was concentrated under reduced pressure to give Compound 113 (200 mg, 215.34 μmol, 95.01% yield, 98.94% purity, 1.0 HCl) as a light yellow oil.

LCMS [M+1]⁺=882.8

¹H NMR (400 MHz, CHLOROFORM-d) δ=12.17 (br s, 1H), 7.20-7.14 (m, 2H), 7.13-7.08 (m, 2H), 4.94-4.76 (m, 2H), 3.01-2.83 (m, 7H), 2.68 (t, J=7.2 Hz, 2H), 2.28 (dt, J=3.6, 7.6 Hz, 4H), 2.19-2.08 (m, 2H), 1.67-1.59 (m, 4H), 1.59-1.54 (m, 4H), 1.54-1.47 (m, 6H), 1.38-1.18 (m, 60H), 0.94-0.84 (m, 12H)

### General Procedure for the Preparation of Compound 114:

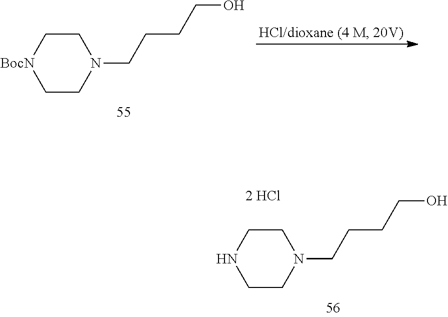 [SMILES: CC(C)(C)OC(=O)N1CCN(CCCCO)CC1 ; OCCCCN1CCNCC1]

Compound 55 (1 g, 3.87 mmol, 1 eq) was dissolved in HCl/1,4-dioxane (4 M, 20.00 mL, 20.67 eq), the mixture was stirred at 25° C. for 2 hours. Take one drop of the reaction mixture and basified with NaHCO₃, TLC (Dichloromethane:Methanol=10:1) showed starting material was consumed and main spot with larger polarity was detected. The reaction mixture was dried in vacuum to give Compound 56 (900 mg, crude, 2.0HCl) as white solid.

LCMS [M+1]⁺=159.2

¹H NMR (400 MHz, DMSO-d₆) δ=11.88-11.44 (m, 1H), 9.98-9.38 (m, 2H), 3.70-3.54 (m, 4H), 3.45-3.36 (m, 6H), 3.29-3.22 (m, 1H), 3.16-3.07 (m, 2H), 1.81-1.64 (m, 2H), 1.50-1.39 (m, 2H).

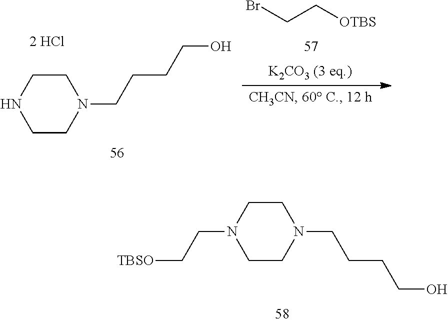 [SMILES: CC(C)(C)[Si](C)(C)OCCBr ; OCCCCN1CCNCC1 ; CC(C)(C)[Si](C)(C)OCCN1CCN(CCCCO)CC1]

To a solution of Compound 56 (900 mg, 3.89 mmol, 1 eq, 2 HCl) in CH₃CN (20 mL) was added K₂CO₃ (1.61 g, 11.68 mmol, 3 eq), then Compound 57 (838.25 mg, 3.50 mmol, 0.9 eq) was added in. The resulting mixture was heated to 60° C. and stirred for 12 hours. LCMS (ET94708-201-P1A) showed starting material was remained and main peak with desired mass was detected. The reaction mixture was filtered, filter cake was rinsed with EtOAc (15 mL), filtrate was diluted with H₂O (10 mL) and extracted with EtOAc (20 mL*2), the organic phase was washed with brine (10 mL), dried over anhydrous Na₂SO₄, filtered and concentrated in vacuum. The residue was combined with batch ET94708-199 (100 mg scale), then purified by column chromatography (SiO₂, DCM:MeOH (7 M NH₃ in MeOH)=100/0 to 100/1) to give Compound 58 (680 mg, 2.15 mmol, 55.17% yield) as a yellow oil.

LCMS [M+1]⁺=317.3

¹H NMR (400 MHz, CHLOROFORM-d) δ=6.52-6.21 (m, 1H), 3.75 (t, J=6.4 Hz, 2H), 3.57 (br d, J=5.0 Hz, 2H), 2.78-2.22 (m, 12H), 1.68 (br s, 4H), 0.89 (s, 9H), 0.05 (s, 6H)

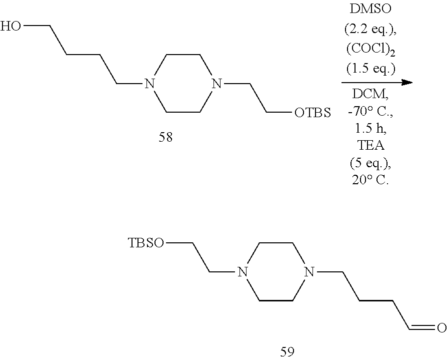 [SMILES: CC(C)(C)[Si](C)(C)OCCN1CCN(CCCCO)CC1 ; CC(C)(C)[Si](C)(C)OCCN1CCN(CCCC=O)CC1]

To a solution of (COCl)₂ (469.13 mg, 3.70 mmol, 323.54 μL, 1.5 eq.) in DCM (10 mL) was added DMSO (423.55 mg, 5.42 mmol, 423.55 μL, 2.2 eq.) dropwise at −70° C. The mixture was stirred at −70° C. for 0.5 hour. Compound 58 (780 mg, 2.46 mmol, 1 eq.) in DCM (3 mL) was added dropwise at −70° C. The mixture was stirred at −70° C. for 1.5 hours. TEA (1.25 g, 12.32 mmol, 1.71 mL, 5 eq.) was added at −70° C., the mixture was stirred at 20° C. for 1 hour. The reaction was adjusted pH to 7-8 by aqueous NaHCO₃ (20 mL). Then extracted with Dichloromethane (10 mL×3) and concentrated under reduced pressure to give a residue. The residue was purified by column chromatography (SiO₂, Dichloromethane:Methanol=1:0 to 20:1) to give Compound 59 (500 mg, 1.59 mmol, 69.44% yield, 100% purity) as a yellow oil.

LCMS [M+1]⁺=315.3

¹H NMR (400 MHz, CHLOROFORM-d) δ=9.77 (s, 1H), 3.76 (t, J=6.4 Hz, 2H), 2.69-2.38 (m, 12H), 2.36 (br t, J=6.8 Hz, 2H), 1.86-1.79 (m, 2H), 0.90 (s, 9H), 0.06 (s, 6H)

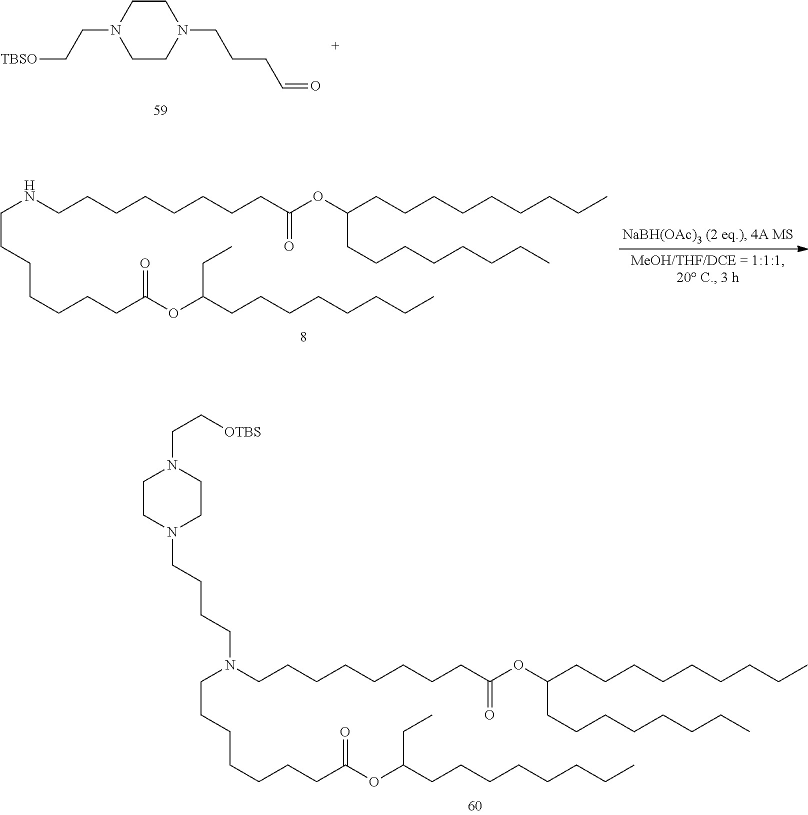 [SMILES: CC(C)(C)[Si](C)(C)OCCN1CCN(CCCC=O)CC1 ; CCCCCCCCCC(CCCCCCCC)OC(=O)CCCCCCCCNCCCCCCCC(=O)OC(CC)CCCCCCCC ; CCCCCCCCCC(CCCCCCCC)OC(=O)CCCCCCCCN(CCCCCCCC(=O)OC(CC)CCCCCCCC)CCCCN1CCN(CCO[Si](C)(C)C(C)(C)C)CC1]

A mixture of Compound 59 (500 mg, 1.59 mmol, 1 eq.), 4A MS (1 g) and Compound 8 (1.03 g, 1.43 mmol, 0.9 eq.) in DCE (5 mL) and MeOH (5 mL) was stirred for 1 hour at 20° C. The mixture was added to a solution of NaBH(OAc)₃ (673.81 mg, 3.18 mmol, 2 eq.) in THF (5 mL), the resulting mixture was degassed and purged with N₂ for 3 times, and then the mixture was stirred at 20° C. for 2 hours under N₂ atmosphere. LCMS showed the reaction was completely. The reaction mixture added NaHCO₃ aqueous (40 mL), and extracted with DCM (40 mL*3), dried over Na₂SO₄, filtered and concentrated under reduced pressure to give a residue. The residue was purified by column chromatography (SiO₂, Dichloromethane:Methanol=I/O to 20/1) to give Compound 60 (500 mg, 489.83 μmol, 30.81% yield, 100% purity) as a yellow oil.

LCMS [M/2+1]⁺=511.1

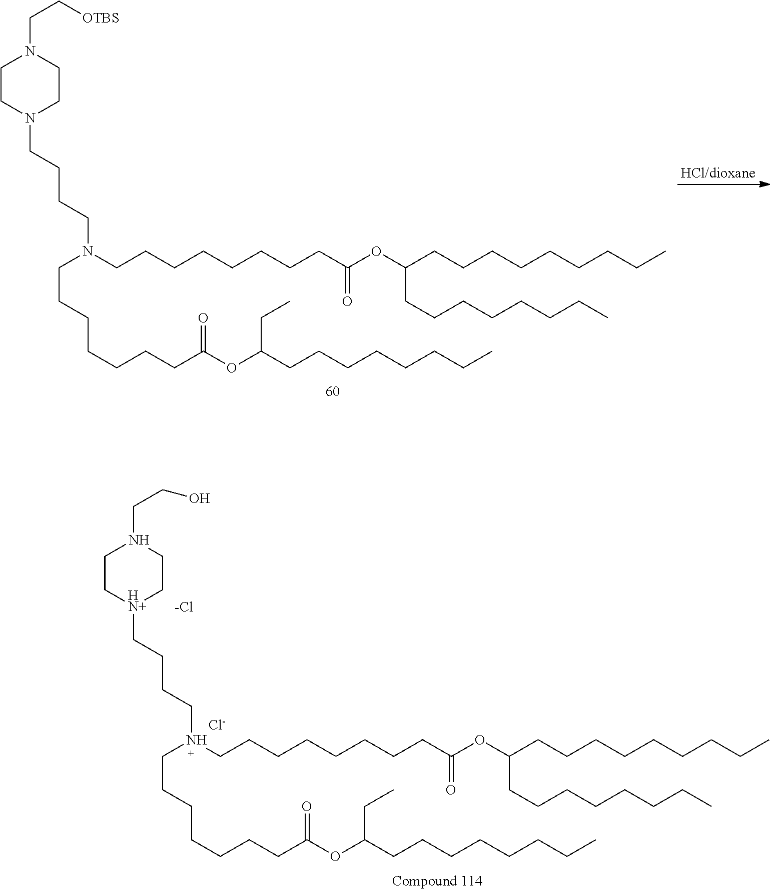

To a solution of Compound 60 (500 mg, 489.83 μmol, 1 eq.) in dioxane (5 mL) was added HCl/dioxane (4 M, 5.00 mL, 40.83 eq.) at 20° C. The mixture was stirred at 20° C. for 1 hour. LCMS showed the reaction was completely. The reaction mixture added aqueous solution of NaHCO₃ (5 mL) and extracted with DCM (10 mL*3), the organic phase was dried over Na₂SO₄, filtered and concentrated under reduced pressure to give Compound 114 (free) (400 mg, 439.50 μmol, 89.72% yield, 99.6% purity) as a yellow oil.

To a solution of Compound 114 (free) (400 mg, 441.26 μmol, 1 eq.) in DCM (4 mL) was added HCl/dioxane (1 M, 3.09 mL, 7 eq.) at 20° C. The mixture was stirred at 25° C. for 2 hours. The reaction mixture was concentrated under reduced pressure to give a residue. The residue was purified by prep-HPLC (column: Welchrom CSH C18 100*30*7; mobile phase: [H₂O (0.02% HCl)-ACN:THF=1:1]; gradient: 45%-85% B over 14.0 min) to give Compound 114 (350 mg, 345.77 μmol, 78.36% yield, 2.9 HCl) as white solid.

TOF-HRMS [M+1]⁺=906.8571

¹H NMR (400 MHz, CHLOROFORM-d) δ=12.31-11.78 (m, 1H), 11.72-11.07 (m, 1H), 10.79-10.35 (m, 1H), 4.90-4.76 (m, 2H), 4.36 (br d, J=2.0 Hz, 6H), 4.17-4.04 (m, 4H), 3.97 (br s, 4H), 3.61-3.38 (m, 3H), 3.25-3.02 (m, 4H), 2.35-2.22 (m, 4H), 2.21-1.87 (m, 4H), 1.86-1.67 (m, 4H), 1.62 (br s, 3H), 1.58-1.47 (m, 8H), 1.43-1.21 (m, 52H), 0.94-0.81 (m, 12H).

### General Procedure for the Preparation of Compound 115:

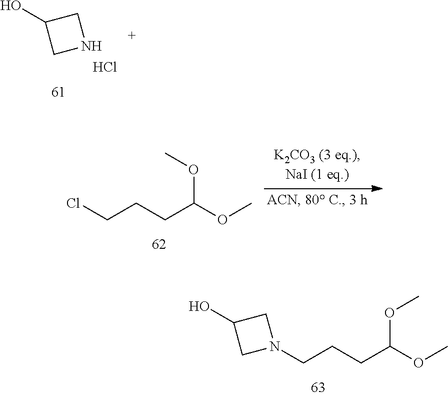

A mixture of Compound 61 (6 g, 54.77 mmol, 1 eq., HCl), Compound 62 (8.36 g, 54.77 mmol, 1 eq.), K₂CO₃ (22.71 g, 164.30 mmol, 3 eq.) and NaI (8.21 g, 54.77 mmol, 1 eq.) in ACN (60 mL) was degassed and purged with N₂ for 3 times, and then the mixture was stirred at 80° C. for 3 hours under N₂ atmosphere. TLC indicated ˜60% of Compound 61 remained, and one major new spot with lower polarity was detected. The reaction was adjusted to pH 7-8 by aqueous solution of NaHCO₃ (50 mL). Then extracted with Ethyl acetate (20 mL×3), the organic phase was concentrated under reduced pressure to give a residue, which was purified by column chromatography (SiO₂, Dichloromethane:Methanol=40:1 to 20:1) to give Compound 63 (1.6 g, 8.45 mmol, 15.44% yield, 100% purity) as a yellow oil.

LCMS [M+1]⁺=190.3

¹H NMR (400 MHz, CHLOROFORM-d) δ=4.42 (quin, J=5.6 Hz, 1H), 4.36 (t, J=5.6 Hz, 1H), 3.67-3.57 (m, 2H), 3.37-3.27 (m, 6H), 2.93-2.82 (m, 2H), 2.63-2.28 (m, 3H), 1.64-1.55 (m, 2H), 1.48-1.34 (m, 2H)

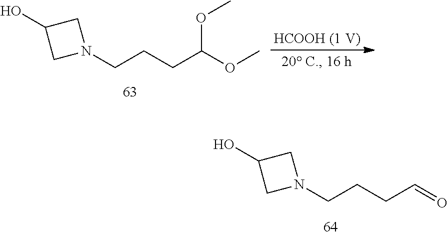

A mixture of Compound 63 (1.6 g, 8.45 mmol, 1 eq.) in HCOOH (461.54 mg, 8.45 mmol, 1.6 mL, 88% purity, 1 eq.) was degassed and purged with N₂ for 3 times, and then the mixture was stirred at 25° C. for 16 hours under N₂ atmosphere. TLC indicated ˜20% of Compound 63 was remained, and one major new spot with larger polarity was detected. The reaction mixture was concentrated under reduced pressure to give Compound 64 (1.59 g, 8.33 mmol, 98.48% yield, 99.08% purity, FA) as yellow oil.

LCMS [M+1]⁺=144.2

¹H NMR (400 MHz, CHLOROFORM-d) δ=10.74-10.34 (m, 1H), 10.24-9.72 (m, 2H), 6.05-5.05 (m, 1H), 4.94-4.71 (m, 1H), 4.66-3.86 (m, 4H), 3.72-3.42 (m, 2H), 2.26-2.11 (m, 1H), 2.09-1.94 (m, 2H)

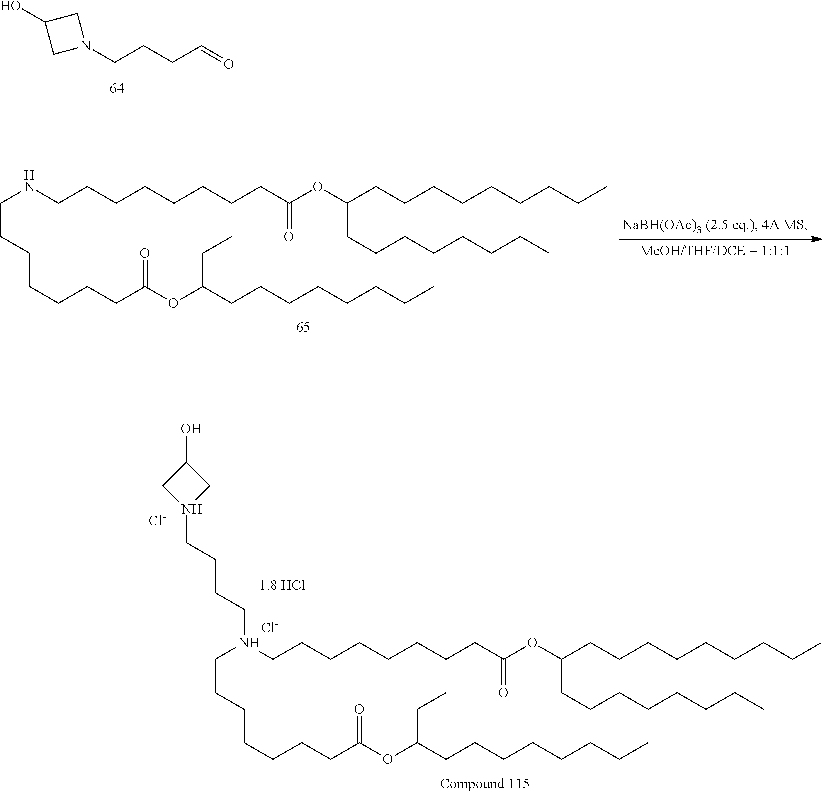 [SMILES: O=CCCCN1CC(O)C1 ; CCCCCCCCCC(CCCCCCCC)OC(=O)CCCCCCCCNCCCCCCCC(=O)OC(CC)CCCCCCCC ; [Cl-] ; CCCCCCCCCC(CCCCCCCC)OC(=O)CCCCCCCCN(CCCCCCCC(=O)OC(CC)CCCCCCCC)CCCC[NH+]1CC(O)C1 ; [Cl-]]

A mixture of Compound 64 (500.00 mg, 2.64 mmol, 3 eq., 1 FA) and Compound 65 (636.17 mg, 880.86 μmol, 1 eq.) in MeOH (2 mL) and DCE (2 mL) was stirred for 1 hour with 4A MS (600 mg) in 20° C. for 30 min, then NaBH(OAc)₃ (504.07 mg, 2.38 mmol, 2.7 eq.) was added in, the mixture was degassed and purged with N₂ for 3 times, and then the mixture was stirred at 20° C. for 2 hours under N₂ atmosphere. LCMS showed ˜17.16% of Compound 65 remained. Several new peaks were shown on LCMS and
40.59% of desired MS was detected. The reaction was adjusted to pH about 7-8 by aqueous solution of NaHCO₃ (10 mL) then extracted with Dichloromethane (10 mL*3), the organic phase was dried over Na₂SO₄ and concentrated under reduced pressure to give a residue, which was purified by column chromatography (SiO2, Dichloromethane:Methanol=1:0 to 40:1) to give crude product (260 mg), and then the crude product (260 mg) was repurified by prep-HPLC (column: X-Select CSH Phenyl-Hexyl 100*30 5u; mobile phase: [H₂O (0.02% HCl)-ACN:THF=1:1]; gradient: 40%-80% B over 16.0 min) to give Compound 115 (100.02 mg, 109.31 μmol, 12.41% yield, 100% purity, 1.8 HCl) as a white solid.

TOF-HRMS [M+1]⁺=849.8049

¹H NMR (400 MHz, CHLOROFORM-d) δ=12.01-11.43 (m, 1H), 11.21-10.88 (m, 1H), 4.94-4.78 (m, 2H), 4.72 (br s, 1H), 4.54 (br d, J=2.4 Hz, 1H), 4.40-4.23 (m, 1H), 4.21-3.98 (m, 2H), 3.54-3.22 (m, 2H), 3.20-2.82 (m, 6H), 2.29 (q, J=7.2 Hz, 4H), 2.21-1.84 (m, 4H), 1.75 (br s, 4H), 1.60 (br d, J=13.6 Hz, 4H), 1.58-1.45 (m, 8H), 1.41-1.18 (m, 52H), 0.96-0.79 (m, 12H).

### Example 2: In Vivo Expression Evaluation of Compound 100 in a Lipid Nanoparticle Formulation

In this example, messenger RNA molecules encoding hEPO proteins were formulated in LNPs for delivery in vivo. The lipid nanoparticle formulations comprised a lipid composition of a lipid:helper lipid:cholesterol:DMG-PEG2k as indicated in Table 1. The lipid mixture in ethanol was mixed with hEPO mRNA in RNA acidifying buffer (10 mM citrate, pH 4) at a N-to-P ratio of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into 20 mM HEPES (pH 7.8), 50 mM sodium chloride, 15% (w/v) sucrose solution via PD-10 desalting column. The neutralized particles were concentrated using 100 kDa AMICON® Ultra centrifugal filters and sterile filtered using 0.2 um syringe filters. LNPs were then stored at −80 C until use. Prior to in vivo dosing, LNPs were thawed at room temperature and diluted 2-fold with 20 mM HEPES (pH 7.8), resulting in a final dosing buffer of 20 mM HEPES (pH 7.8), 25 mM sodium chloride, 7.5% (w/v) sucrose. Resulting LNPs were characterized by dynamic light scattering (DLS) measurement for measuring its hydrodynamic diameter and polydispersity index (PDI) and the Zeta potential was measured using a Zetasizer (Malvern). Encapsulation efficiency (EE %) and total RNA concentration were quantitated using a fluorescence based Ribogreen assay, and the apparent pKa of the LNPs was measured by a fluorescence based TNS assay. The formulation and measurements for each of the LNPs are shown in Table 1 below.

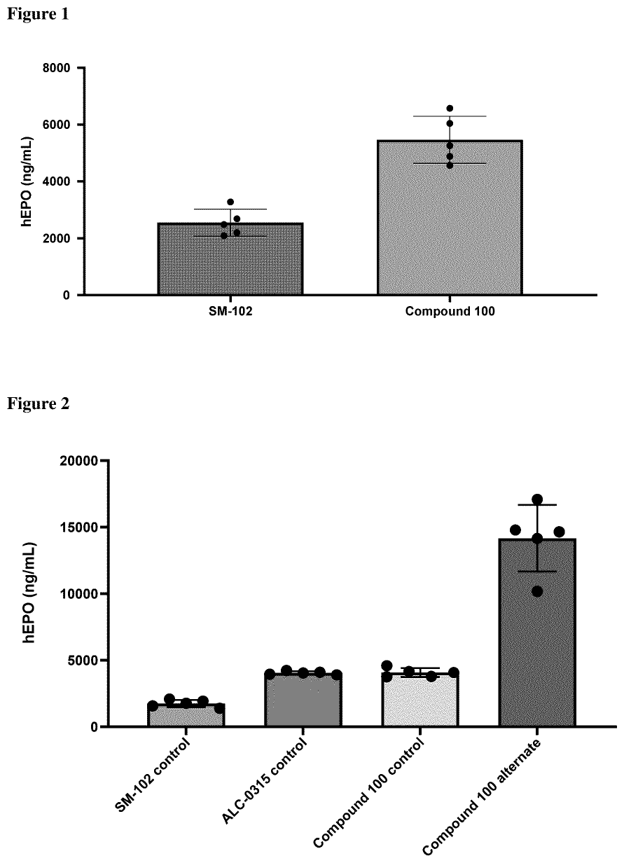

The in vivo studies were performed in C57BL/6 female mice at 6 to 8 weeks weighing in at approximately 20 g. The LNPs formulated with different ionizable lipids at 0.2 mg/kg of hEPO mRNA were administered by tail vein injection and animals were euthanized at 6 h post-administration for blood serum sample collection. The hEPO levels from the samples were analyzed and cross-compared by enzyme-linked immunoassay (ELISA) according to manufacturer's protocol. The hEPO expression levels for the LNP shown in Table 1 was compared to a control LNP comprising SM-102 (DC Chemicals Cat. No. DC52025) (FIG. 1).

TABLE 1
LNP formulations for in vivo study

| | Lipid:DSPC:Cholesterol:DMG-PEG2k | | N:P | Size | | Zeta potential | Encapsulation | |
|---|---|---|---|---|---|---|---|---|
| Ionizable Lipid | (mol %) | mRNA | ratio | (nm) | PDI | (mV) | Efficiency % | pKa |
| SM-102 | 50:10:38.5:1.5 | hEPO | 6 | 79.1 | 0.205 | — | 91.5 | 6.1 |
| Compound 100 | 50:10:38.5:1.5 | hEPO | 6 | 99.5 | 0.087 | — | 75.6 | 5.8 |

### Example 3: In Vivo Expression Evaluation of Compound 100 in an Alternative Lipid Nanoparticle Formulation

In this example, formulations containing variations in the lipid nanoparticle composition of lipid:helper lipid:cholesterol:DMG-PEG2k were compared. The lipid nanoparticle formulations comprised a lipid composition of a lipid:helper lipid:cholesterol:DMG-PEG2k as indicated in Table 2. Messenger RNA molecules encoding hEPO proteins were formulated in LNPs for delivery in vivo. The lipid mixture in ethanol was mixed with hEPO mRNA in RNA acidifying buffer (10 mM citrate, pH 4) at a N-to-P ratio of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into 20 mM HEPES (pH 7.8), 50 mM sodium chloride, 15% (w/v) sucrose solution via PD-10 desalting column. The neutralized particles were concentrated using 100 kDa AMICON® Ultra centrifugal filters and sterile filtered using 0.2 um syringe filters. LNPs were then stored at −80 C until use. Prior to in vivo dosing, LNPs were thawed at room temperature and diluted 2-fold with 20 mM HEPES (pH 7.8), resulting in a final dosing buffer of 20 mM HEPES (pH 7.8), 25 mM sodium chloride, 7.5% (w/v) sucrose. Resulting LNPs were characterized by dynamic light scattering (DLS) measurement for measuring its hydrodynamic diameter and polydispersity index (PDI) and the Zeta potential was measured using a Zetasizer (Malvern). Encapsulation efficiency (EE %) and total RNA concentration were quantitated using a fluorescence based Ribogreen assay, and the apparent pKa of the LNPs was measured by a fluorescence based TNS assay. The formulation and measurements for each of the LNPs are shown in Table 2 below.

The in vivo studies were performed in C57BL/6 female mice at 6 to 8 weeks weighing in at approximately 20 g. The LNPs formulated with different ionizable lipids at 0.2 mg/kg of hEPO mRNA were administered by tail vein injection and animals were euthanized at 6 h post-administration for blood serum sample collection. The hEPO levels from the samples were analyzed and cross-compared by enzyme-linked immunoassay (ELISA) according to manufacturer's protocol. The hEPO expression levels for each of the LNPs shown in Table 2 were compared to control LNPs comprising SM-102 (DC Chemicals Cat. No. DC52025) and ALC-0315 (DC Chemicals Cat. No. DC42537) (FIG. 2).

TABLE 2
Alternative LNP formulations for in vivo study

| | Lipid:DSPC:Cholesterol:DMG-PEG2k | | N:P | Size | | Zeta potential | Encapsulation | |
|---|---|---|---|---|---|---|---|---|
| Ionizable Lipid | (mol %) | mRNA | ratio | (nm) | PDI | (mV) | Efficiency % | pKa |
| SM-102 | 50:10:38.5:1.5 | hEPO | 6 | 79.4 | 0.290 | −6.1 | 92.0 | 6.4 |
| ALC-0315 | 46.3:9.4:42.7:1.6 | hEPO | 6 | 74.2 | 0.492 | −8.0 | 81.0 | 5.9 |
| Compound 100 | 50:10:38.5:1.5 | hEPO | 6 | 89.9 | 0.093 | −15.1 | 73.0 | 6.1 |
| Compound 100 | 51.5:2.7:44.5:1.3 | hEPO | 6 | 102.9 | 0.059 | −14.8 | 74.1 | 6.1 |

### Example 4: In Vivo Expression Evaluation of Compound 100 in an Alternative Lipid Nanoparticle Formulation

In this example, formulations containing variations in the lipid nanoparticle N-to-P ratio were compared. Messenger RNA molecules encoding hEPO proteins were formulated in LNPs for delivery in vivo. The lipid nanoparticle formulations comprised a lipid composition of a lipid:helper lipid:cholesterol:DMG-PEG2k as indicated in Table 3. The lipid mixture in ethanol was heated to 25-30 C, and mixed with hEPO mRNA in RNA acidifying buffer (10 mM citrate, pH 4, 18-20 C) at an N-to-P ratio of 10 for Compound 100 and an N-to-P ratio of 6 for SM-102 using a multi-inlet vortex mixer with Chromtech LD-100 HPLC pumps at a combined flow rate of 150 mL/min (45 mL/min for aqueous buffer+RNA, 30 mL/min for ethanol+lipid mix, and 37.5 mL/min for each of the (2) aqueous buffer streams). Lipid nanoparticle intermediate mixed product was then immediately diluted with an in-line addition of 10 mM citrate, pH 4 (10-15 C) at a combined flowrate of 300 mL/min (150 mL/min for the mixed product and 150 mL/min for the aqueous buffer).

The resulting particles were held at room temperature for 60 minutes and neutralized by adding 200% (v/v of product) addition of Dulbecco's phosphate buffered saline (15-20 C) to the mixed product at a flowrate of 200 mL/min, while stirring at 200 rpm, followed by a 20% (v/v of product) addition of 100 mM phosphate pH 8.0 (15-20 C) to the mixed product at a flowrate of 200 mL/min, while stirring at 200 rpm. The neutralized particles were concentrated 25× on TFF using a T-series Cassette with Omega (mPES, 100 kD) membrane at a loading of 0.5 g/m\hat{ }2 RNA:membrane area. After concentration, particles were buffer exchanged with 8 diavolumes of 20 mM HEPES pH 7.8, then concentrated to system holdup (approximately 0.3 mg/mL). TFF processed particles were stored overnight at 4 C, then concentrated via 100 kDa AMICON® Ultra centrifugal filters to 1.5 mg/mL. Particles were filtered with a Sartopore Platinum 25, 0.2 um filter with a loading of 15-20 g/m\hat{ }2 RNA:membrane area, at a constant flowrate of 2 mL/min. Samples were then characterized for total RNA concentration and diluted with excipients to dose concentration and target buffer composition for storage (20 mM HEPES, 25 mM NaCl, 7.5% Sucrose). Samples were stored at −80 C in sterile vials. Final characterization was performed after thawing of the frozen material. LNPs were characterized by dynamic light scattering (DLS) measurement for measuring its hydrodynamic radius and polydispersity index (PDI). Encapsulation efficiency (EE %) and total RNA concentration were quantitated using a fluorescence based Ribogreen assay. Zeta-potential was measured using an electrophoretic mobility assay. The measurements for each of the LNPs are shown in Table 3 below.

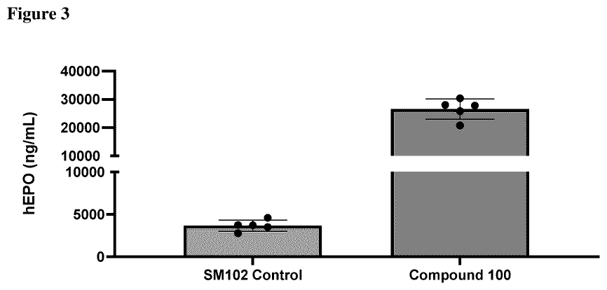

The in vivo studies were performed in C57BL/6 female mice at 6 to 8 weeks weighing in at approximately 20 g. The LNPs formulated with different ionizable lipids at 0.2 mg/kg of hEPO mRNA were administered by tail vein injection and animals were euthanized at 6 h post-administration for blood serum sample collection. The hEPO levels from the samples were analyzed and cross-compared by enzyme-linked immunoassay (ELISA) according to manufacturer's protocol. The hEPO expression level for the Compound 100 LNP shown in Table 3 was compared to a control LNP comprising SM-102 (DC Chemicals Cat. No. DC52025) (FIG. 3).

TABLE 3
Alternative LNP formulations for in vivo study

| | Lipid:DSPC:Cholesterol:DMG-PEG2k | | N:P | Size | | Zeta potential | Encapsulation | |
|---|---|---|---|---|---|---|---|---|
| Ionizable Lipid | (mol %) | mRNA | ratio | (nm) | PDI | (mV) | Efficiency % | pKa |
| SM-102 | 50:10:38.5:1.5 | hEPO | 6 | 91.1 | 0.070 | −5.5 | 95.5 | — |
| Compound 100 | 50:10:38.5:1.5 | hEPO | 10 | 76.4 | 0.050 | −6.2 | 92.3 | — |

### Example 5: In Vivo Expression Evaluation of Selected Target Compounds in a Lipid Nanoparticle Formulation

### Formulation of LNPs

In this example, messenger RNA molecules encoding hEPO proteins and messenger RNA molecules encoding fLuc proteins were formulated in LNPs at a 1:1 mass ratio for delivery in vivo. The lipid nanoparticle formulations comprised a lipid composition of a lipid:helper lipid:cholesterol:DMG-PEG2k as indicated in Table 4. The lipid mixture in ethanol was mixed with combined hEPO and fLuc mRNA in RNA acidifying buffer (10 mM citrate, pH 4) at a total N-to-P ratio of 6 using a microfluidic device (Precision NanoSystems, Inc.) at a combined flow rate of 10 mL/min (7.5 mL/min for aqueous buffer, RNA and 2.5 mL/min for ethanol, lipid mix). The resulting particles were neutralized by buffer exchange into 20 mM HEPES (pH 7.8), 25 mM sodium chloride, 7.5% (w/v) sucrose solution via PD-10 desalting column. The neutralized particles were sterile-filtered using 0.2-um syringe filters. LNPs were then stored at −80 C until use. Prior to in vivo dosing, LNPs were thawed at room temperature. Resulting LNPs were characterized by dynamic light scattering (DLS) measurement to measure hydrodynamic diameter and polydispersity index (PDI) and the Zeta potential was measured using a Zetasizer (Malvern). Encapsulation efficiency (EE %) and total RNA concentration were quantitated using a fluorescence-based Ribogreen assay, and the apparent pKa of the LNPs was measured by a fluorescence-based TNS assay. The formulation and measurements for sample LNPs and control are shown in Table 4 below.

### In Vivo Studies

The in vivo studies 1 and 2 were performed in C57BL/6 female mice at 6 to 8 weeks weighing approximately 20 g. The LNPs formulated with different ionizable lipids at 0.2 mg/kg of hEPO mRNA and 0.2 mg/kg of fLuc mRNA were administered by tail vein injection. Animals were euthanized 6 h post-administration for blood serum sample collection. The hEPO levels in samples were analyzed and cross-compared by enzyme-linked immunoassay (ELISA) according to manufacturer's protocol. The hEPO expression levels for the LNPs shown in Table 5 include control LNPs comprising either ALC-0315 or LP-01 (DC Chemicals Cat. No.: DC42537 and Cat. No.: DC31000, respectively).

The in vivo studies were performed in C57BL/6 female mice at 6 to 8 weeks weighing approximately 20 g. The LNPs formulated with different ionizable lipids at 0.2 mg/kg of Luc mRNA were administered by tail vein injection. Animals at 6 h post-administration were dosed with 100 μL of d-luciferin IP and euthanized 20 minutes later for IVIS imaging. The liver, spleen, lung, kidneys, pancreas and femurs were removed and placed onto black paper for imaging. Tissues were imaged initially at 1 minute, 10 bin, FOV 15. Image times and binning were then adjusted accordingly.

TABLE 4
LNP formulations for in vivo study

| | | | | | | | hEPO |
|---|---|---|---|---|---|---|---|
| | Lipid:DSPC:Cholesterol:DMG-PEG2k | | | Size | | Encapsulation | Expression |
| Ionizable Lipid | (mol %) | mRNA | N:P ratio | (nm) | PDI | Efficiency % | (ng/mL) |
| ALC-0315 | 50:10:38.5:1.5 | hEPO/fLuc | 6 | 72 | 0.202 | 89.7 | 10259.6 |
| Compound 101 | 50:10:38.5:1.5 | hEPO/fLuc | 6 | 75.2 | 0.134 | 68 | 119.878 |
| Compound 102 | 50:10:38.5:1.5 | hEPO/fLuc | 6 | 87.4 | 0.072 | 91 | 4496.4016 |
| Compound 103 | 50:10:38.5:1.5 | hEPO/fLuc | 6 | 79.2 | 0.252 | 81 | 98.5884 |
| Compound 104 | 50:10:38.5:1.5 | hEPO/fLuc | 6 | 78.9 | 0.234 | 84 | 1660.3822 |
| Compound 106 | 50:10:38.5:1.5 | hEPO/fLuc | 6 | 60.6 | 0.163 | 93 | 219.5004 |
| Compound 108 | 50:10:38.5:1.5 | hEPO/fLuc | 6 | 131.1 | 0.153 | 95.9 | 517.4008 |
| Compound 109 | 50:10:38.5:1.5 | hEPO/fLuc | 6 | 115.2 | 0.075 | 70 | 1163.7852 |
| Compound 110 | 50:10:38.5:1.5 | hEPO/fLuc | 6 | 126.5 | 0.312 | 94.9 | 104.07 |
| Compound 112 | 50:10:38.5:1.5 | hEPO/fLuc | 6 | 63.7 | 0.115 | 96.5 | 79.5506 |
| Compound 113 | 50:10:38.5:1.5 | hEPO/fLuc | 6 | 67.9 | 0.396 | 72.9 | 32.0916 |
| Compound 114 | 50:10:38.5:1.5 | hEPO/fLuc | 6 | 126.3 | 0.133 | 97.8 | 59.951 |
| Compound 115 | 50:10:38.5:1.5 | hEPO/fLuc | 6 | 141.4 | 0.078 | 99.2 | 150.772 |

TABLE 5
Selected fLuc Expression in Six Tissues for LNPs containing Compound
101 to 115 compared to control LNPs containing ALC-0315

| | Liver Total | Spleen Total | Pancreas Total | Kidney Total | Lung Total | Bone Marrow |
|---|---|---|---|---|---|---|
| Ionizable Lipid | Flux (p/s) | Flux (p/s) | Flux (p/s) | Flux (p/s) | Flux (p/s) | Total Flux (p/s) |
| ALC-0315 | 5.01E+10 | 2.80E+08 | 2.84E+07 | 1.76E+08 | 2.84E+07 | 4.26E+07 |
| Compound 101 | 4.02E+05 | 1.14E+05 | 6.07E+04 | 4.96E+04 | 6.26E+04 | 2.90E+05 |
| Compound 102 | 1.34E+10 | 1.03E+08 | 6.45E+06 | 6.54E+06 | 6.21E+06 | 1.24E+07 |
| Compound 103 | 7.23E+07 | 4.71E+05 | 6.11E+04 | 1.11E+05 | 5.32E+04 | 1.61E+05 |
| Compound 104 | 6.26E+09 | 4.98E+07 | 2.08E+06 | 7.39E+06 | 4.62E+06 | 1.51E+07 |
| Compound 106 | 1.62E+08 | 8.93E+06 | 1.11E+04 | 2.04E+06 | 1.48E+05 | 2.26E+05 |
| Compound 108 | 4.25E+09 | 1.46E+08 | 7.47E+06 | 4.50E+06 | 2.12E+06 | 2.40E+06 |
| Compound 109 | 1.26E+09 | 4.56E+08 | 4.03E+07 | 3.14E+06 | 4.28E+07 | 3.20E+07 |
| Compound 110 | 1.16E+08 | 4.00E+06 | 1.37E+05 | 3.39E+05 | 1.04E+05 | 2.15E+05 |
| Compound 112 | 2.41E+07 | 1.31E+08 | 1.73E+07 | 3.21E+05 | 3.67E+06 | 7.83E+05 |
| Compound 113 | 9.48E+04 | 3.87E+04 | 3.18E+04 | 3.06E+04 | 2.46E+04 | 4.40E+04 |
| Compound 114 | 5.35E+06 | 3.86E+07 | 9.06E+04 | 1.94E+05 | 9.41E+05 | 1.29E+05 |
| Compound 115 | 8.86E+05 | 6.63E+07 | 7.78E+04 | 5.26E+04 | 1.55E+07 | 3.05E+05 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A lipidoid of formula (I), or a salt thereof; 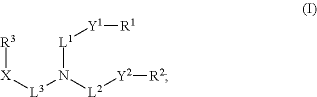
wherein:
each of L¹, L², and L³ represents —(CH₂)ₘ;
m is independently for each occurrence an integer selected from 1-20;
Y¹ is 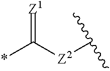
and * represents the connection to L¹;
Y² is 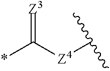
and * represents the connection to L²;
Z¹, Z², Z³, and Z⁴ are each independently O, S, or NH;
X is optionally substituted saturated or unsaturated cycloalkylene, heterocyclylene, heteroarylene, or arylene;
R¹ and R² are each independently optionally substituted linear (C₄-C₂₀)alkyl, branched (C₄-C₂₀)alkyl, linear (C₄-C₂₀)alkenyl, branched (C₄-C₂₀)alkenyl, linear (C₄-C₂₀)alkynyl, or branched (C₄-C₂₀)alkynyl, optionally wherein two substituents, together with the intervening atoms, form an aryl or 5-7 membered cycloalkyl ring; and
R³ is hydrogen, hydroxyl, hydroxyalkyl, amido, or an optionally substituted linear C₂-C₁₀ alkyl or branched C₃-C₁₀ alkyl.

2. The lipidoid of claim 1, wherein at least one of Y¹ or Y² is *—(C═O)O—.

3-6. (canceled)

7. A lipidoid of formula (II), or a salt thereof; 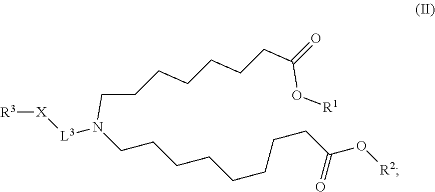
wherein:
L³ is —(CH₂)ₘ—;
m is an integer selected from 1-10;
X is an optionally substituted saturated or unsaturated cycloalkylene, heterocyclylene, heteroarylene, or arylene;
R¹ and R² are each independently optionally substituted linear (C₄-C₂₀)alkyl, branched (C₄-C₂₀)alkyl, linear (C₄-C₂₀)alkenyl, branched (C₄-C₂₀)alkenyl, linear (C₄-C₂₀)alkynyl, or branched (C₄-C₂₀)alkynyl, optionally wherein two substituents, together with the intervening atoms, form an aryl or 5-7 membered cycloalkyl ring; and
R³ is hydrogen, hydroxyl, hydroxyalkyl, amido, or an optionally substituted linear C₂-C₁₀ alkyl or branched C₃-C₁₀ alkyl.

8-9. (canceled)

10. A lipidoid of formula (V), or a salt thereof; 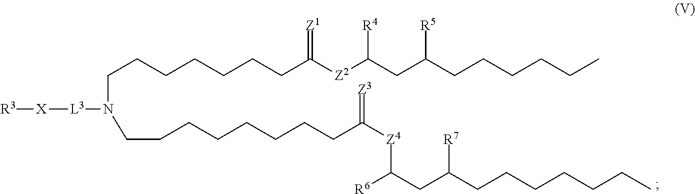
wherein:
L³ represents —(CH₂)ₘ—;
m is an integer selected from 1-10;
each of Z¹, Z², Z³, and Z⁴ are independently O, S, or NH;
X is an optionally substituted saturated or unsaturated cycloalkylene, heterocyclylene, heteroarylene, or arylene;
R³ is hydrogen, hydroxyl, hydroxyalkyl, amido, or an optionally substituted linear C₂-C₁₀ alkyl or branched C₃-C₁₀ alkyl.
R⁴ and R⁶ are each independently H, linear C₂-C₁₀ alkyl or branched C₃-C₁₀ alkyl;
R⁵ is H, linear C₂-C₁₀ alkyl, or branched C₃-C₁₀ alkyl,
or R⁵ and R⁴, taken together with the intervening atoms, form an aryl or a 5- to 7-membered cycloalkyl; and
R⁷ is H, linear C₂-C₁₀ alkyl, or branched C₃-C₁₀ alkyl,
or R⁷ and R⁶, taken together with the intervening atoms, form an aryl or a 5- to 7-membered cycloalkyl.

11-16. (canceled)

17. The lipidoid of claim 1, wherein X is a four to seven-membered heterocyclylene.

18. (canceled)

19. The lipidoid of claim 1, wherein —X—R³ is selected from:

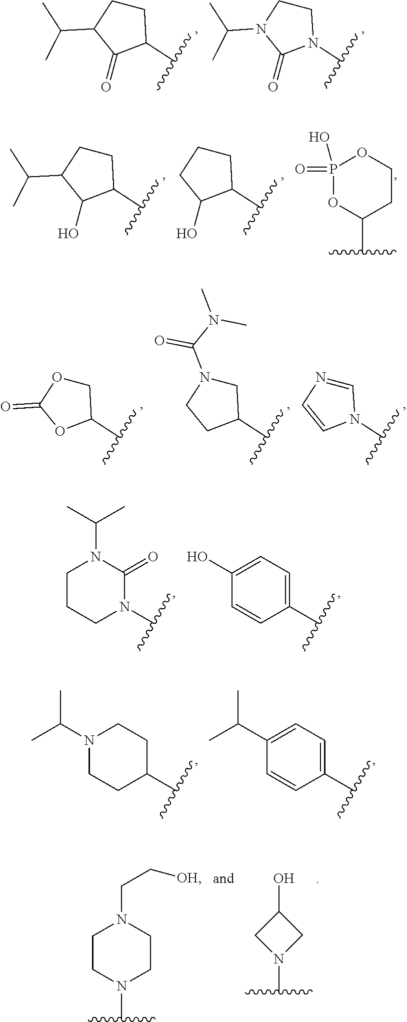

20. The lipidoid of claim 1, wherein L³ is —(CH₂)₃—.

21. (canceled)

22. The lipidoid of claim 1, wherein m is independently for each occurrence an integer selected from 2-10.

23-30. (canceled)

31. The lipidoid of claim 1, wherein R¹ and R² are each independently branched (C₁-C₂₀)alkyl.

32. The lipidoid of claim 1, wherein R¹ is 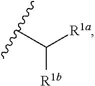
and R^{1a} and R^{1b} are each independently (C₂-C₁₈)alkyl, wherein R¹ contains from 8 to 20 total carbon atoms.

33-34. (canceled)

35. The lipidoid of claim 1, wherein R² is 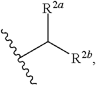
and R^{2a} and R^{2b} are each independently (C₂-C₁₈)alkyl, wherein R² contains from 8 to 20 total carbon atoms.

36-40. (canceled)

41. The lipidoid of claim 1, wherein Z¹, Z², Z³, and Z⁴ are each O.

42. The lipidoid of claim 1, selected from the following table:

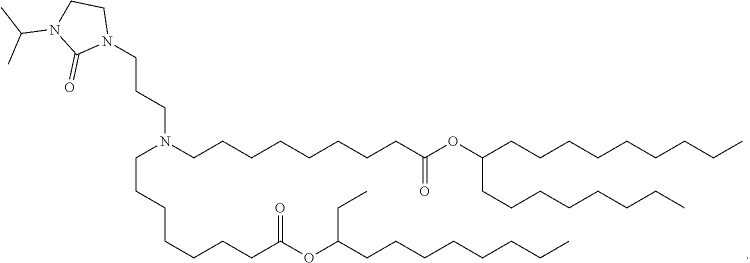 [SMILES: CCCCCCCCCC(CCCCCCCC)OC(=O)CCCCCCCCN(CCCCCCCC(=O)OC(CC)CCCCCCCC)CCCN1CCN(C(C)C)C1=O]

43. The lipidoid of claim 1, selected from the following table:

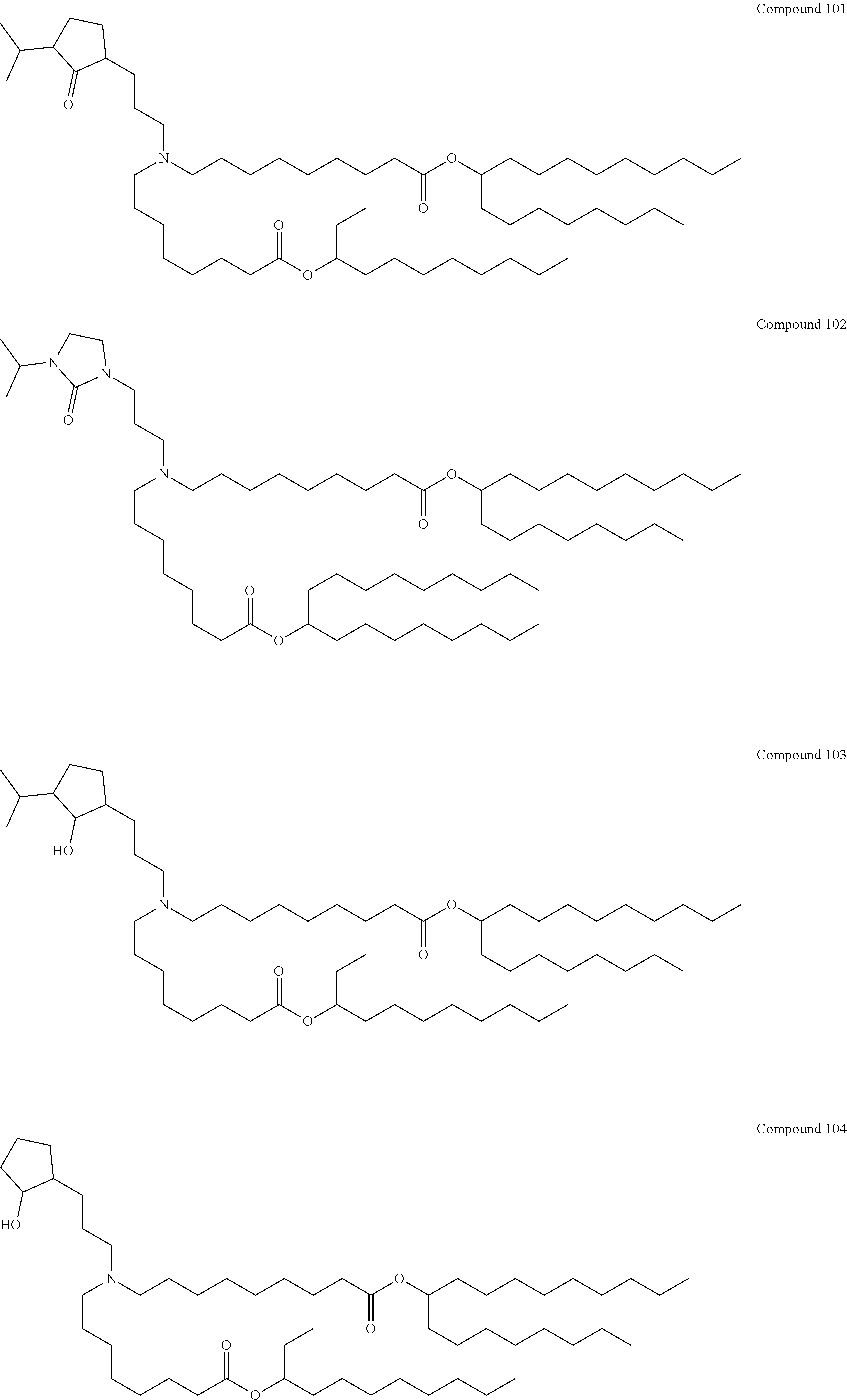

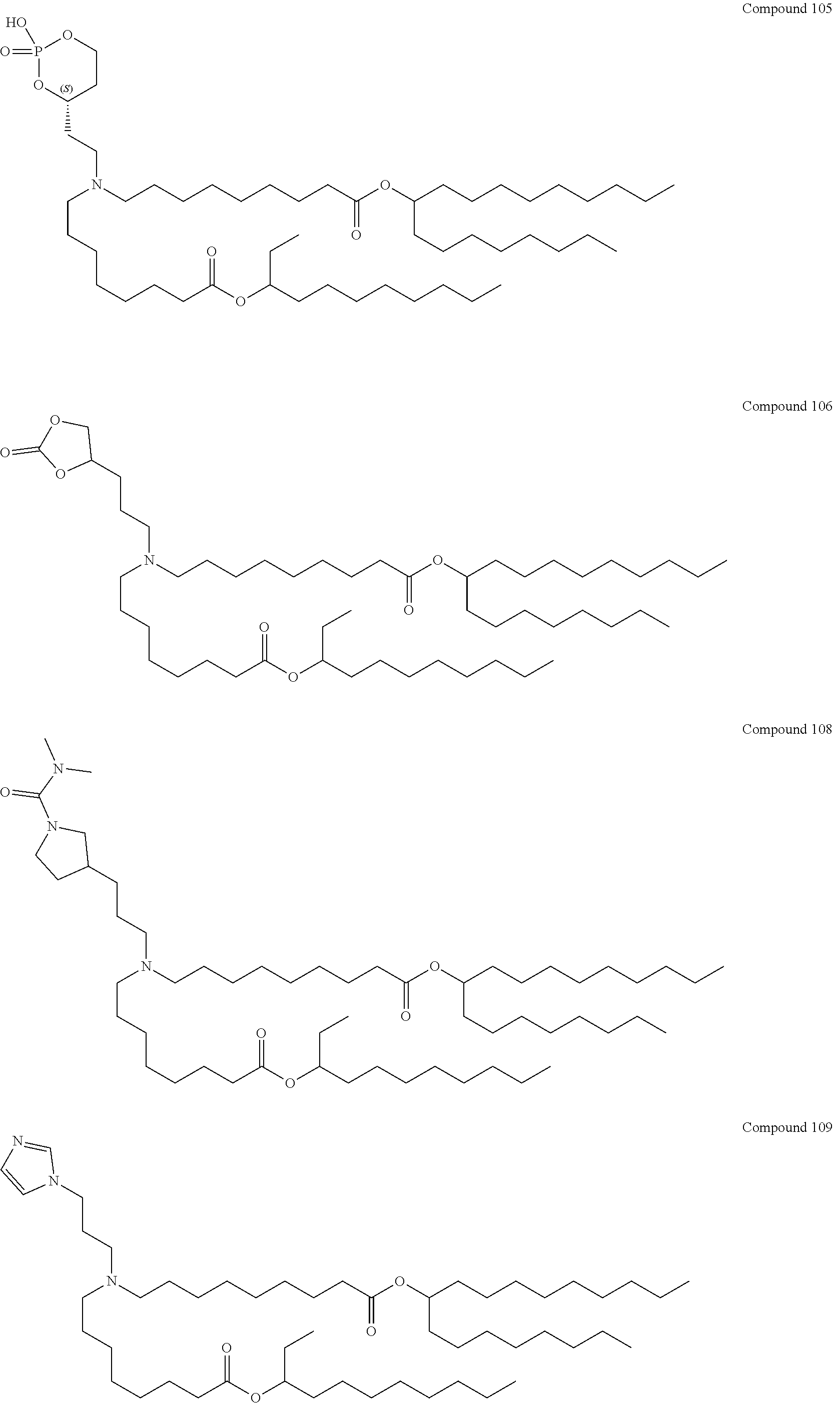

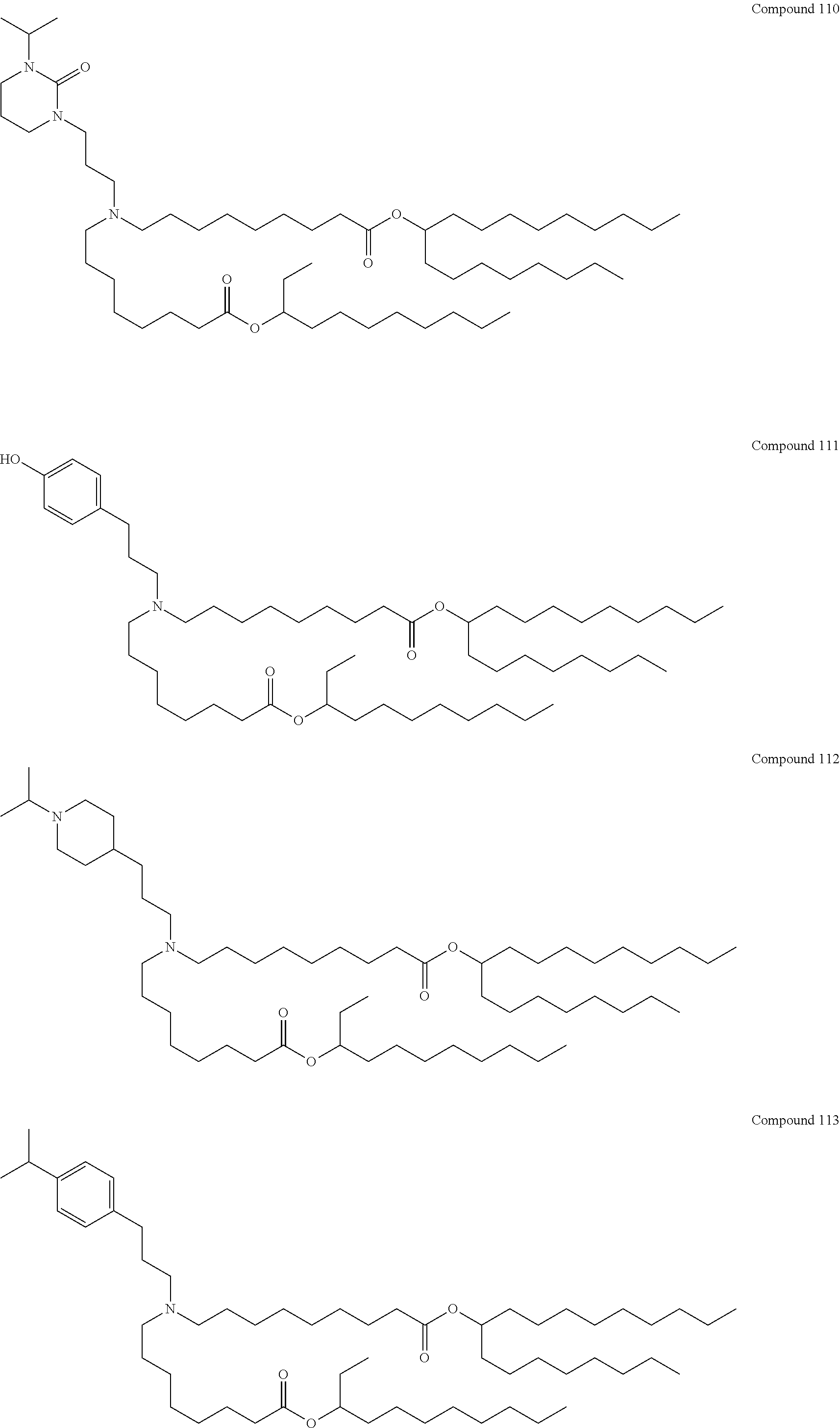

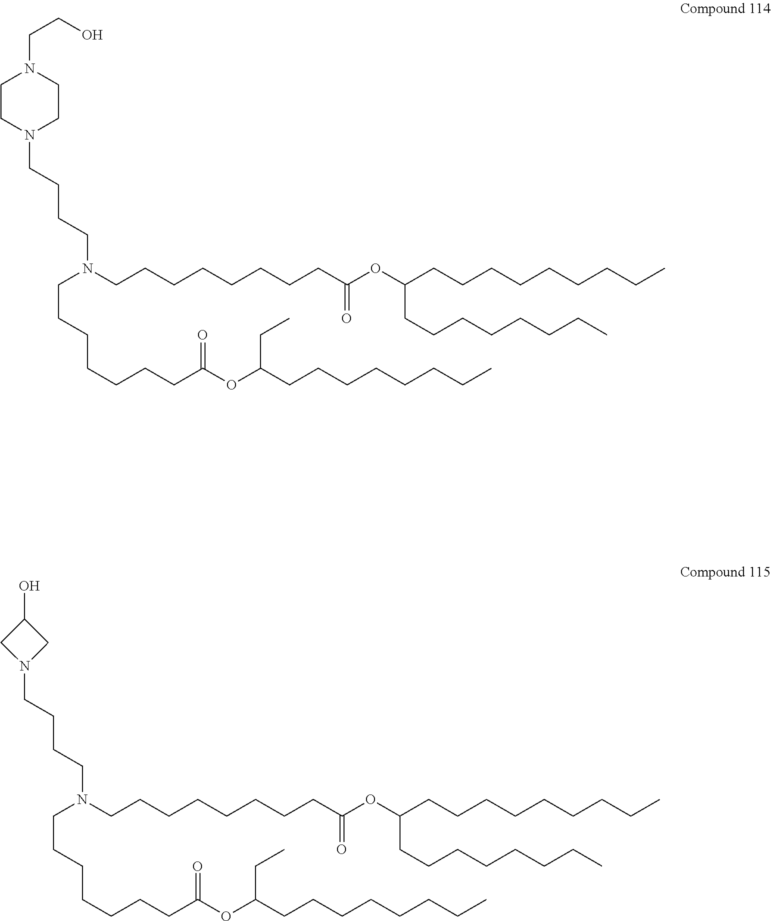 [SMILES: CCCCCCCCCC(CCCCCCCC)OC(=O)CCCCCCCCN(CCCCCCCC(=O)OC(CC)CCCCCCCC)CCCCN1CCN(CCO)CC1 ; CCCCCCCCCC(CCCCCCCC)OC(=O)CCCCCCCCN(CCCCCCCC(=O)OC(CC)CCCCCCCC)CCCCN1CC(O)C1]

44. A nanoparticle composition, comprising a plurality of a lipidoid of claim 1, or a salt thereof.

45-48. (canceled)

49. The nanoparticle composition of claim 44, further comprising a PEGylated lipid, a sterol, and a phospholipid.

50. The nanoparticle composition of claim 44, further comprising a therapeutic agent.

51-63. (canceled)

64. The nanoparticle composition of claim 49, wherein the lipid nanoparticle comprises:
i) the lipidoid in an amount from about 40 mol % to about 55 mol %;
ii) the phospholipid in an amount from about 1.0 mol % to about 3 mol %;
iii) the sterol in an amount from about 35 mol % to about 50 mol %; and
iv) the PEGylated lipid in an amount from about 0.5 mol % to about 3 mol %.

65-66. (canceled)

67. A pharmaceutical composition, comprising a nanoparticle composition of claim 44, and a pharmaceutically acceptable carrier.

68. A method of delivering a therapeutic agent, comprising administering to a subject in need thereof an effective amount of the nanoparticle composition of claim 50.

69. (canceled)
